SEQUENCE LISTING (20 sequences)

SEQ ID NO: 1 - protein, 732 aa, Anaerolinea thermophila
MDMSSFVRETPNHFSLPRRIARLGDLAYNLWWAWNPEALRLFARIDRTLWEQVYHNPVAFLRRVERRHLNAATNDRYFLESYDAVIYAFDQYMNEENTWFKRNYPQLTNQQIAYFSFEFGLHESLPVYAGGLGVLAGDHLKEASDLGLPLAGVGFYYTQGYFSQRITEDGWQETRNLYLNYEELPVVPLVHPEGGPLMVSVTLAGRKVFARILQIQVGRVPLFLLDTDVESNTLADRSLTARLYTSDQEVRISQEILLGIGGLRALRLLGYSPTVWHMNEGHSAFLTIQRLREYVAQGFSLEEAKERIRKTNIFTTHTPVPAGNDEFPLWMMDKYFSHVYNELKMDRDLFIDLGRNMQEWGETFSMPVLALRLSEKCNAVSELHGRVSRRMWHKLWADKKENDIPIFHITNGVHVGTWMARRWRLLFERYLGMDWMDNLDDPEMWMMIDSIPDAEIWAVRRHLKNKLVHYAIERARRHWVTGEAHPVQVVAGGVLLEPYSLTIGFARRFATYKRANLIMRDVDRLIRIITNADMPVQIIFAGKAHPADEPGKLLIQQVYRAVKDSRAGGRLVFLEDYDMNLARLLVQGVDVWMNTPRRPNEASGTSGMKAAMNGVLNFSVLDGWWREGFNGKNGWAIGEEKDYTDPNEQDERDAQSLYATLENEIIPLYYGSRAGDDIPHAWIERIKESMRTLIPQFSTRRMLKEYLQQAYLPAMEAAVQMEALPGEGKRSN

SEQ ID NO: 2 - protein, 690 aa, Thermobaculum terrenum
MEIRNKLNDLAYNLKWSWTPSTAELFSHIDSENWQKTKHNPVLLLKKISDERLRMLLADKSFVDRVNQEHDSLQRYLEANNTWFSENYGARDFSVAYFSAEFGLVECLPIYSGGLGVLAGDHLKSASDLGIPLVGVGLLYKNGYFFQKIDENGWQQEEYPDINYLHTPLTLVTDMSGNRVIGEVDVADAVVKFQIWKARVGRVDLYLLDTNLEENQAPYRDITDKLYGGDSEKRIQQEIVLGIGGIRALRMLGLYPQVYHMNEGHSAFLVLERMREMIQDMGKSFEEALETVRSTNVFTTHTPVAAGHDYFSIDLIERYLQSYINKLGISFEQLLALGRKRPEDENELFCMTVLALKSSSKNNGVSKLHGRVSQEMWRSLWPELPVEDVPIGYVTNGVHAPTWLSSSLRDILSDSEGNIDWQKILEVDDELLWRLHKELKRQLIELVNSRSKIGKLGNLTEDVLTIGFARRFATYKRATLLFSDEEKLAAILNNPQHPVQILFSGKAHPRDNGGKELIQKIVQLSKREPFLGKIAFIEDYDLEIARHLVRGCDVWLNNPVRPMEASGTSGMKASMNGVLNASTLDGWWAEAWESSNKGSDGFGWAIGDGKVYESPEVQNEVESRAIYELLEASIIPLFYDRDEKGIPRKWVKMMKEAISGLGPKFTTDRMLKEYVEGFYMVDRTDDKILV

SEQ ID NO: 3 - protein, 811 aa, Thermincola potens
MFKNKEEFKQAFLEKLHTLYGECLDEASDLDKYNALGSMIRDHIAKYWTQTNKQYRKKEVKQVYYFSMEFLLGRLLGANLINLSAKKMCEEALKELGIDLNLLENIEPDAGLGNGGLGRLAACFLDSLASLSLPGHGCGIRYKYGFFEQRIVDGYQVELPDNWLRDGNVWEIRKADNAVEVRFYGNVRVEEQDGRTVFIHENYEPIIAVPYDTPIIGYERNTVNTLRLWSAEPAIKDFDFSSFSRGDYVKAVEYRSAVESISQILYPDESHYKGRELRLKQQYFFVSAGLQCIVRRYKKYHGSLKCFHKKIAIHVNDTHPVLAIPELMRILIDEEGMGWDEAWDITVKTISYTNHTILTEALEKWPVDMFRNLLPRIYMIVHEINERFCRELWNKYPGDWDRIHKMAIIADGQVKMAHLAIVGSHSINGVSKIHTEILKNQEMKNFYEVYPHKFNNKTNGVTHRRWLLKANPALANLISETIGANWIKYPIDLISLEKYRNDTAFLDKLAQIKQRNKAQLAKYIKDKYNISVDLHSIFDVQVKRMHAYKRQLLNVLHIMDLYNRLRENPDLDIVPRTFIFGAKASPSYVQAKTTIKLINTLASMINNDKRIKDKIKVIFLENYRVTLAEMIFPAADLSEQISTAGKEASGTGNMKFMMNGAVTIGTMDGANIEICEVVGRDNIFIFGLTAEEVANYYHNGGYCSWDVYAKDSRVKTVVDQLVNGFFPVAGEEFRVLYDDLTHNNDPFFVLKDFASYADTQLEVDKTFRDKYKWQKMSLVNIAHSGKFSSDRTITEYATQIWNIKPEIITNP

SEQ ID NO: 4 - protein, 852 aa, Thermodesulfobacterium geofontis
MEFKKFFVYPRIPEKLKKLLDLSNNLWFTWNYDALSIFYKIDAETFRRLKYNAKKFLYYLPKPIFQRLEKDERFLMDLEDIWETFEEYKEYKHPLIESANFTSEEIIAYFSTEFGFHPVLPVYAGGLGILAGDMIIGASDLGLPLIGIGLLYKNGYFRQKIDPLAKTQIEEADEVEVFLNFLQEVKTEKDEPLIINLEILDKPVKVKVWKLEVGRNICYFLDTDIYENSPEMRDILRYLYPGEPEKRIQQEIILGLGGYYALKAMGIKPKLYHLNEGHSAFVIFARLKDLIKEEGLSLEEAKMLIKETTIFTTHTPVISGNEHFPHELVKKYLFDLLEDVLDLETREKLFEEGFIGKDKTIFWLPALAIRNSSYVNAVSRIHQNTTKQMWNPLFEKFISEEVPIYYITNGVHWRWLSEPFYNLLKKYLGTHFIYMSEEDTGWEEILKIPGEEIWEAHKRNKYRLIAYLKKVLEEEYFKIRHLGKETRVFKFPTAHHLIITCARRVTGYKRNTLILYNKEKLAEILRNTDTILLFAGKAHPKDEEGKKMIKEILEFREQYNFYDKVIFLENYDIHLARYLVWGSDVWLNTPYRPMEASGTSGIKASMNGVLHLSVLDGWWPEGYTGNNGWAIHPKEGLPPYNAFEANQIYNLLENEIIPLFFERDEEGIPKNWIKMMKQAIYTACKHFSINRVLLEYTQKFYIPALENFKILTENNYAFLRELINKKNTILEKWKDIKILNVYDNLVGRNLFEEDKLELTVEIDLAGLSPDLIEVQVVFISEIYCVVAPGVEEEIERRLEVYPIPFKEYKDNKAIFYGVYPLYAHGLKQYSIRIVPKNVFIKRTSPDLIKWKN

SEQ ID NO: 5 - protein, 831 aa, Thermosipho africanus
MMDVSKKLKDEKYRTSKKVKDLKENFIDHLNLTLGKDFKNATMWDKFYALSLVVKDRVLERWLKTQKKYYESNDVKRVYYLSIEFLMGRLLYNNILNLKIDKEIKKAMDEIGLSLDELSEIEPDAGLGNGGLGRLAACFLDSIATLSYPGYGYGIRYEYGIFKQLIKDGFQVEVPDDWLKNGNPWEIERKDRAVKVKFFGRTESYKDKEGNTRFRWVDTYDVIALPYDTPVVGYGNDVANTLRLWSAKPITEFDFDNFQKGNYVKAVESQAIAGAISKVLYPNDAFYAGRELRLKQEYFFVSASIQDIIRRFKSQFGNNFDIFPEKNVIQLNDTHPALAIPELMRILVDEEFLPWEKAWEITTKTFAYTNHTVMPEALEKWEVHLLERLLPRHLEIMYEINARFLDNVSKYYPGNIEKIRNVSIFEEGHVKQARMANLSVVGSFSINGVSKLHTEILKERVFKDFYDIWPEKFNNKTNGITQRRWLLQSNPELSKLITDTIGDEWIVNLDHLKNLEKYADDKVFLNEFYKVKQNNKIRLSNYIKKELNIDVNPDSIFDVQVKRLHEYKRQLLNVMHIIYLYQTLKENPEQDIYPRTFIFGAKAAPGYRMAKLIIKLINSVADVINNDNEIADKIKVVFVPNYNVSLAEIIIPAANVSEQISTAGKEASGTGNMKFALNGALTIGTLDGANIEIKECVGDENIFIFGLTAEQVAKLKESRLYNPYEIYLRNENIRKILDAINNGYFNKNDPELFKDIFQSLLFGLNGAQADEYMLLADFDSYKTRHKEIDFIYRDKYRWNKKALLNVARVGMFSSDRTIREYARDIWKVKSI

SEQ ID NO: 6 - protein, 916 aa, Spirochaeta africana
MKGYSFVHPRGDFVLPHADRTNGLYFPLANEAGYMAAITPTGGGDAKIDQNHFLLPPAAVVDLHNTRFTRTIWVRFDDGSCWSATGSSHEQKAAEPELHTVEAGLLWHKTIRKHAARPLTAEIVSFVPNDEVTAEVVRFTLRNTGASPLRVLPTAVVPLFGRSADSLRDHRHVTALLNRVEITSQGVLLQPSMSFDERGHSVNTTVYAVMGRRGDGSLPTGCFADLDSFIGEGGDLERPAVIYSPEHADAVRLSPGDVVEGSEALGAVQFDQVEIAPGAEIQLYCLAGVFAHTDGAARAAERYCNPAGFEAALAANREFWDAKLNTVSCTTGNSEFDAWMRWVSLQPILRRLYGCSFLPYHDYGRGGRGWRDLWQDCLALLLMEPDSVRESLLQNLAGVRFDGTNATIIGSRPGEFVADRNNITRVWMDHGAWPLMTIQLYIDQTGDSGFLFESQTYFSDPQWGRASRKLDATRGDTLLYDQTGGVVRGSVFEHLLLQQLSAFFHVGDHNSILLEGADWNDGLDMAADRGESVAFTGQYAANLAWLAGVLEQVSAEVTEIALHHEILQLLDRYAGTPVDYDDPAAKRARLQSFFDSCQQGPSGEMVNVAAVDLAADLRAKSDAMIDHLRNREWIQTPDGHGWFNGYYDNDGERVEGGGTDSPRMTLTGQVFQVLGGVADEQQVDRIVAAADAHLWDDAVGGYRLNTDFRELKLNMGRCFGFAYGHKENGAMFSHMAVMFANALYARGRSQAGWRTLEAIFNAASRFSASRMYPGIPEYFDPKGRGLYPYLTGSASWYLLTVVTRMFGVRGHYGDLVLDPQLQAAQWGELDSVSVCTYFRQRQLQIVYRNPEGLDVGEYRVTAVVADTGELAFTLRDGAAVIDGAAIDRVAMDCTGPEQAPTVLRVELGRRDNGAGV

SEQ ID NO: 7 - protein, 956 aa, Clostridium saccharolyticum
MTACFCPVFVERKVKTEGRRCAEEVKIMTFSAKYLNSVIHPLGIYFKKITGMKGWKIGMKYEFKGNRGNFYMENPDLTSYLYFPLANESGVMSCVSPDLGGDSKMDQNSFLMPPVSCENLHNDKSSRNVWCRIDGSTLWSLTGRSSWQQAQKFSENKEPMAVEAGFMHHKITRTSDQIGIKGEISSLVPATGESVELMKIQIENTSSQYKSFQIITAIPLYARSADNIRDHRHVTSLLHRIKTKSSGVIVTPTMTFDERGHKPNFRAYGVFGGSESQGPVGYFPVLEDFIGEGGSLENPKALQERQLKPEKENFKTGGYEALGGLCFQECRVGPKEKITYVVAMGYGNTEDELIQNCSQFLSERAFDRCWEQTAEYWNHKVNVRLKTGNPAFDLWMRWVSFQPMLRRIYGCSFLPHHDYGKGGRGWRDLWQDCLALLMMNPAGVRQMLIDNFGGVRMDGTNATIIGNRQGEFVADRNNITRVWMDHGVWPFLTTELYIHQTGDLKILLEKNSYFKDMQVCRGEDKDIQWKTEDGERLRDGRGRIYSGTVLEHLLVQNLTSFYDVGEYNHIRIRGADWNDALDMAAQRGESVAFTCMYGYNLKGLAVFLEELKAQGVAEFEMAEEVRLLLAEDPAVYDDIRRKQEILKDYCRTCSHHVSGKRAVVKAVDLQADLLSKASWIYHHIRQTEWIDSKEGYGWYNGYYDNSGNRVEGETKAGVRMMLTSQVFSLMSGVAEDPQVKDIVKAADALLYRKDIGGYRLNTDFHEVKMDLGRMFGFAYGHKENGAVFSHMATMFGNALYRRNASGEGYHALLSLFEHCSNSEKSRIYPGIPEYVDAGGRGMYHYLTGAASWYLVTVVTQMFGVRGHYGNLIFKPQLIKEQFDEHCQAWVSMEFAGKSLKVLYKNPRNLDPDAYRITSVTVNGIPCHCPGEEWIIKREELLKLPDQELHTIMVELN

SEQ ID NO: 8 - protein, 916 aa, Halorhabdus tiamatea
MAKRHLHDIDTNVDPWAFVDDVGTFRLADPQRSSYLYFPLVNEAGIMSAITPKLHGDINASQHEFLTPPTAVENLHNKRTARNFWVCEHGVGPWSAAGNSATQIPEERPGGAESVSMEGGLLYHEITRENDERGLRAEITNFAPVRDDMVELMRVAVTNTGEKTKTITPTAAVPIYGRSADNIRDHRHVTSLLQETWTEEYGVLVRPTLSFDERGHTRNETTYGVLGTEADGTAPESFYPVVEDFIGEGGNLEWPEAVVEDLAGVSAGTEVDGYESLGGLRFETTALEPGETAEYVVIQTIWTDDPDPATLVERYGSSEAVAEELAACQDHWREKSSAVEFDTGDDTFDQWMRWVTLQPVFRRLFGNSFLPYHDYGRGGRGWRDLWQDILSLLLTEPDNVTDLLYNNFAGVRFDGSNATIIGDEPGEFTADRNNIPRVWMDHGAWPWLTTRFYLDLSGDLQFLLRDQQYYKDLHVDRASEQDEAWSPEDGTELYTDDGEIYEGTVLEHLLVQHLTQFFNVGEHNVMRLEDADWNDAMDMAPERGESVAFTALYAWNLRDMSDVLDALRAELDVEEIEIARELQTLLDTLSEPVDYDDPEAKQARLDDYLDTWERTVSGEKATVAIEELAADLEQKAEWLYEQLRDQEFIEDEDGHQWFNGYYDDSGRRVEGDHDNGVRMTLTGQVFTLMGGVATDDQADAIVEAADEYLYEPKMRGYRLNTDFDELKTDLGRGFGFAFGHKENGAMFSHMAVMYANALYRRGKVEAGHRVLSGIYEQSKDFEVSRIYPGIPEYFSERGRGMYTFLTGSGSWLLLTTVTEVFGVKGRLGDLRLEPKLLAEQFDDGEAAVTCQFADRRLDVTYHNPENADVGDYEITAVSLNGDSVDFERTDDGVVIDREVVTGLDSEAVHDVDVTLE

SEQ ID NO: 9 - protein, 904 aa, Halothermotrix orenii
MGWEFNNNKGTFSLKGADDNSYLYFPLTNESGIMSSITPTLNGDIKTDQNSFLMQPVSVEDLHNNRSNRNFWLKIDGYGPWSVTGNSARQISMKYTGDKKIETADVEAGFLWHKVIRKNRKLNIKAEITNFVPVNEDQVELMKVTIVNTGQETIKFTPTAAIPVYGRSADNLRDHRHVTSLLHRITTNSNGVIVTPTLSFDERGHQPNNTSYGVFGRCSDSSDPVGFCPVLEDFIGEGGTLDWPRSIVEGNVDFVKPGFKYEGYESIGAIKFGDKELKAGESISYIIVMVIDHNGKDMKKYIDKYCSEKDFDRELNENKKYWQSTIEQLTFYTGDETFDNWMKWVTLQPVLRRIYGCSFLPYHDYGRGGRGWRDLWQDCLALLLMDDKNVRYLLYNNFAGVRIDGSNATIIGNKPGEFIADRNNITRLWIDHGAWPLQTTSLYINMSGDIKFLMEKQSYFKDRIVSFAREIDNRWHQGMGNKLKTEEGQVYHGSILEHILLQNLTVFFNVGEHNNIRLEDADWNDGLDMASEKGESVAFTAFYAGNLFEIVNLLETLRDKENVTEVKLLEEIKVLLDTLYNPVDYNDVTAKNEVLNKYFASCKYNISGNQVTIKIDQLIKDIKRKANWLQEHLRENEYIGDNKGHKWFNGYYDNKGQRVEGRFEKETRMTLTGQVFTTMFGIATGEQVKNIISAADYYLYDDGVGGYRLNTDFKDKDIQLGRCFGFAYGHKENGAMFSHMSVMYANALYRRNFVKEGFKVWHSIYNHCLDFDRSRIYPGIPEYISQRGRGMYSYLTGSASWLLLTFATEVFGVKGYYGDLRLEPKLLKEQFDEEGKATIGIKFAGKRLTITYVNPQLLDYDSYKIDKVMVNDEIIDYYEDKQVTIKREDIEERDEVIDITVRLA

SEQ ID NO: 10 - protein, 912 aa, Paenibacillus sp.
MDRKKIHGFEFTGKDGEFKLQQPDHSSFLYFPLVNEGGMMSTVTPKLHGQATTGHNTFLTPPLSVEDLHNSRASRNFWVYVEGKGAWSAAGNSAKQNAAFYTSSADEAVLEAGFLWHRVTRSNSEMGVTAEITSFVPRGNDKVELMKVTLTNIGTEKITLTPTAVVPLYARSADDLRDHRHVTSLLNRIYTSAYGVEVQPALSFDERGHRVNHTSYGVFGAEGDGSQPVGFFPIQEEFIGEGGSLDWPEAVILNTTPEISKGGGVDREGYEAVGGLRFASITLSPGEKHSYVVVMAIDNDRINVEALMNKYGASEAFDQLLEETKAFWADKVNMVEFHTGDSDADQWMKWVTIQPVLRRLYGNSFLPYHDYGRGGRGWRDLWQDCLALLIMEPAEVRSLLFNNYAGVRIDGSNATIIGQQPGEFIADRNNIPRVWMDHGAWPFLTTMLYLDQSGDLDFLLQEQTYFRDTFMSRCKERDTSWDTSAGSDLRTASGEIYKGTILEHILLQNLVPFFNVGEHNNILLEGADWNDGLDMGADRGESVAFTSFYASNLLDISRMLRTLKTSKGQEKVELAEEMQLLLDTLNERVDYGSISGKHERLDRYYAAAPNKVSGVRVSLDIEKVADDLEQKAEWIFNHLRNNEWVQSENGDGWFNGYYNNDGERVEGEFSEGVRMTLTGQVFPLMGHAAAPEQIPHIIAAVDRNLLDDKIGYRLNSRFGGIQQNLGRAFGFAFGHKENGAMFSHMTIMYGNALYKRGYVKEGHKVLESLYNLSTDFAKARIYPGIPEYINEQGRGMYTYLTGSASWLLLTMVTEVFGVKGKLGDLLLEPKLVKEQFDSEGKASIKTIFADREFEVVYKATGSFNYGEYQIHSVTLNGSEVTLQRGSGSAIIPREDLLALQTEKRHVIEVTLA

SEQ ID NO: 11 - protein, 1086 aa, Rhizobium tropici
MSSGTNRSFQTPRREELGLITLSNTAGLSASALPNGTLFSIDFADQQGGVMINQVLGSPVYGGIGRLYLRIGGAKPATAEIVGPKAAVQFGHGKSGFSWSGETAGVKHTISLRLHPNKTAWFWQVSLENSSNATLSADLVLVQDVGLGDRGFLMNSEAYASQYIDHHIADHSAFGPVIMVRQNLKQGGGRNPWLAQGCLEGAAAYATDAIQLLVPSKGNDGVMVPDFGTSLPSARRQHEVACPTIQSKPLALAAGAKATTTFFGLFIADHPAASSDADLAHLDGLPALQSELAAETISAGQPVRSLLQDAPLAESDDLDQAALDALYPRRMLEEHADGKLLSFFVPDGPHNRHIVLREKERLVARRHGAIVRSGQNMLLDDQTLAATCWMQGIFAAQLTIGNTSFHKLFSVSRDPYNLTRSSGLRILVDLGDGWRLLGVPAAFEMGLSDTRWIYRLPGRTIIVSAIASGDDPAMQWSVSVEGAACRFLVFGQIVLGERDYEAVANIAVDTAGKRISFQPQPSWLWDRYPKAGYHLVTSTPDAFEAIGGDELLYSDSTARNGPFIALKSHPTKALRFAVTGSMTDAQAAEALAARYTTGVESEEMLAPARRFWSHVTRGARIEGDGRDVAAQAVMLPWIAHDAIVHLSVPHGLEQYTGAAWGTRDVCQGPIEFLLAYEHDEEVRQILLTLFSEQYRDRGDWPQWFMLEPYANIRAGEAHGDIVVWPLKALCDYIEATGDIAFLAKTVPWRADDTMQLTSEHATISDHIEKLLDTVRSRFVPGTSLIRYGEGDWNDSLQPADPHLRDWMVSSWTVSLLYEQLVRYANILTLANRNDEAQSLQATAVAMRADFNRLLMRDGIVAGYGVFDPSHDGVELLLHPEDTRTGLHYSLIAMTQPMIGGLFAPEQRHAHMKIIRANLLFPDGVRLMEKPATYSGGPEKLFRRAESSSFFGREIGLMYVHAHLRYCEALALDDDAEAVWAALALANPIAVSGRVEHASLRQRNTYFSSSDAAFEDRYQASAEWTRVKAGDIAVDGGWRIYSSGPGLYTKSLIGNVFGFKRQFGQRIRKPLLPASIGACEVKLDFKA

SEQ ID NO: 12 - protein, 1084 aa, Listeria monocytogenes
MLKEIKKADLSAAFYPSGELAWLKLKDIMLNQVIQNPLENRLSQIYVRAHFGDKIEIYPLLNKDSEVGFNENGVEYRGVVGPFRYSVQMHFHTRGWFYDVIVDGDFEFDLVYLQDLGLAEQAAVRTNEAYMSQYIDYHVTEGATGFTVQARQNQPQNERFPAVQIGALTKIVGYATDGFDIYGTNYKLTSELANLKEKSLPNRVYQYEFAQISIQTELFTNHGETSFYGYVTENQPKASGAPIGNLAELKSNISEQSYQKSTKAILNKHIGTPITGETISDSWLQEKFPDRIQEEKQNAALLSFFTPNYAHVVMREKEAELERPHGSILLDKIDVLNPEATLSATTYMYGAFLSQLVAGNTNMNKWNSHARNPLNILQTSGLRIYIELDSELRLLSVPSVWETSTNYSTWYYQWNDDLITVQTTLTAYSKEAFVTVHSEKGHSYKLVLTNQVTMGTNEYDTTVKKEIKDGIVTYFPADDSPILETYPELQFRVDGTYNELTDERYFAKDYMGTAGLDVFVFEPSDKATFHVQAKLTDEFSKPTEDLEADNKVIRASYDELTAQFHLNHQSTTAEKLNLTVYWYAHQMLVHYASPHGLEQYSGAAWGTRDVSQGPFEFFLATGNKAVLRKLVLTIFSHQYQDTGDWPQWFMFDKYTSIQQEESHGDVIVWPLKIIGDYLEMSGDTEILEEAIPYVDRESKTFTKEQGTLLEHIKLAVKTIEARFMKGTALSNYGDGDWDDTLQPANAQLKKNMVSSWTVALTYQTFKRLAAFLPVGEKYETLANNVQADFAKYMTNDTDVIPGFLYLEEGKAPVWMIHPEDKETNIKYRLIPLTRSVISELVDKKQASRNFEIIGEHLLHPDGVRLMSEPAHYAGGVSTHFKRAEQAANFGREVGLQYVHAHIRYIEALAKIGDKSAWLMLDVINPINIKEVVPNAALRQSNTYFSSSDAAFLDRYQAQNEFSRVKEGSIPVKGGWRIYSSGPGIYLHQLISSVLGIRQTEETLIFDPILPEELDGLECHIELDNYPLDLTFESSDEGCIVVNGEKQPVENAANLYRTGALILPKKNLTTKCSQITIKFQKNNRL

SEQ ID NO: 13 - protein, 1122 aa, Clostridium saccharoperbutylacetonicum
MILDNKIDVFSIKNGSTKVEFLPTGDIHQILVDNIMINQYLGNCIDGSVNNIYIRIHKKEKIEILPLLGVKSNSRFYINEKEAVWTGETEGIVYKITFVLQKEGIYFYEISLEGNGEEIDFIYGQDISLARKNDTLTNELYVSQYLDHTILKGKHGYVVSSRQNQEQEYGFPYIQQGSIDTEIVAYSTDAMQFFGKSYKETNKPEILEKGLPNANYQFEMAYIGMQTQKMNIDGRKNITFYSLIKSNYTHRIEKIDYEEELLSAYELFSSDRQKQKESKELEKIHMKKTFGDPFASEQLLKEELDSYFPSKKFEEYGGDKLLSFFTEKHSHVVMREKELAVERPHGHIILTTVDDEYVSREIISSTNYMYGIFNSHVVVGNTNMHKLISTARGHLNLFKNSGQRIYVKIEGIYHLLTLPAVYEMGMNYSRWFYKIGNDILTITSYSIAKKADIVLEVTSKNGYEYEFIITQQLVMGNQEYDEKIQVIQKDRDLIFIPDEDNLIKEVYPNLYYKMRIEGTEFEMSDDRVFFENNNSYNHTFLTISTKSVNTFRIIISGRIEDNSEEIVEDIGLGSFKENEKQYLDIYKKLMGGFNLSLSGSEQKNIEKINEILWWYTHNAMIHYIAPHGLEQPGGAAWGTRDVCQGPLEYFLSTGHSSLVREIIQIIFENQFYESGEWPQWFMYDKYNFRQYDCHGDVVFWPLKVVGDYIRVTGDNSILKCEVGYRHFKDGVATKNKESILDHIKRSICSIKDRYLYDTNLISYGGGDWDDTLQPANEELKQSLVSAWTVTLAYQVIKLLAEEIGKVDVSYGKELVQMYMDIKESFEKYLIKDGVIPGFVYLEDKEHMEYMLHPTDKKIGIQYRLLPMTRSIISELVDKNQAEKNIAIIDRELMHPDGVRLMNCPAFYKGGVSETFKRAEQASNVGREIGLQYVHAHIRYIEAMAKVGDANRAWDGVMRINPINISEKVSNAELRQSNCYFSSSDGEFATRYDFQENFEKLRTGDVNVKGGWRIYSSGPGIYVNQIITNLLGIRMNNNLLILDPVIPKELDNLKFDFHVNHIPVTFVYHINKQGESITKVLLNGKKMELSYIENAYRSGGCRVQYELLKDNFSSKNTIDIYIG

SEQ ID NO: 14 - protein, 834 aa, Acholeplasma brassicae
MIFNNLKNDTGFYIPMYNKLGFKSYVTPRLLGDIKLDYHTYLLEPTTEIDLTSSFFSRHVSFYVDGKPHFLNGNSESQQTDKIDVELGLLYQKVTRHNKKFQLDVTTFVPLDALMESNVITYQNTTSKTQRLKVIVATPLYGRSADNLRDHRHVTSLLNRVEVLENCVVLNPTLSFDERGHKKNELNYGVYASSPQLSVEGYYPSLEDYVKNGSLQFPKGLDSLVQTGDYVEGKEALGGISFKTIEVKSQEVITLYFNLGINHSKEQLISTFNKYSQSKTIAIAFEEVKTYFTKEVSKLQFHMKNLEYSEKLNYIPIQPILRRFQGNSYLPHHDYGKGGRGWRDLWQDLISMIMYNDSECRSLLLNNFAGVRIDGSNATIIGDKAGEFLADRNNIVRVWSDHAAWPLLTTKMYIDETGDLSFLLETQTYFDDQFTHYTKKTKTKKSTNHQLKIQNDTIYGTVLEHLILQNVVAINNVGSNGFVRLEDADWNDGLDMASKKGETIAFTHFYINNLLVLAELIQKLETPKIELFESLTKLILNKNYSLDIFFDDVHDFTDNKVSIDKEILASELTKKATLVLKQINKYAFMREGGLQSYIDNDGNFLDSSSTLSLTGQTMALLNQTLSKTQAKKVSNVTKKHLFDRMIGGYRLNSNYQDVKMNMGRAYGFAYGTKENGAVFSHMSLMYAYGLYQYNFVKQGREAYQTLVNQAFKDKSVVCLGIPEYFNNEGIGKYLYLTGSATWLLKLLRTEVFGIELSFGTLRLNPKLSKDDFIDSIAVIKTYVHNKPLQVTYHNPKKLEYGDYRIASIQMNGKEVINEFQSIEGDLEVYLDEIV

SEQ ID NO: 15 - protein, 912 aa, Paenibacillus stellifer
MSTQTQTWTFKGSEGDFELKDPHLTSYLYFPLVNEAGMMSSITPSLHGDIKSGHNTFLSEPVSAESLHNSRAARNFWVHNQEFGPWSCTGNSAVQHASRLDKETEGGDKVTLEAGLLWHKVIRNNTRIGLRAETVSFVPAGADRVELMKVTLTNTGNRKLTLTPTAAVPLYARSADDLRDHRHVTSLLHRIYTSAGGIEVQPSLSFDERGHRVNKTAYSVLGSEGDGGKPAGFFPIAERFIGEGGNYDWPEAIVRNDVPHSGPGEALEGFEAVGAIRFAEAVLQPGESKSYIVAMAITDDRMDEDRLWKTYMSEASFDHLLGACKAYWREKADAVQFHSANKEFDLWMKWVTLQPILRRLCGNSFMPHHDYGRGGRGWRDLWQDCLALMIMEPEEVRQLLSDNFAGVRIDGSNATIIGRLPGEFVADRNNIPRVWMDHGAWPFLTVLLYLNQSGDLNFLLQEQTYFRDAFVNRCRERDDSWTPESGNLLRTTNGEIYRGTILEHMLLQHLTPFYLVGEHNHMRLEGADWNDGLDMAADRGESVAFTAFYAGNLRDLASVLRKLERRTGTAAAKLAEEMLVLLDSLGEPADYDSVSAKHELLNRYFERIAGGVSGRKTAISLVSLAEDLERKASWIENHIRTSEWVTSESGSSWFNGYYNNDGERVEGDHPEGVRMTLTGQVFPIMSGVAAEEQIPSVIQAVDRHLLDEEIGYRLNSRFGGIQQNLGRAFGFAFGHKENGAMFSHMTVMYANALYKRGFVKEGKAVLDSIYSLCADFPKSRIYPGIPEYINEQGRGMYHYLTGSASWLLLTELMEVYGVKGDLGDLLLQPKLSGEQFDEAGKAAVVTQFAGRRLRVVYINEERKEYGDRQAGSCLLNGKPARVEPAAGGCLLSRALIKSLPEEGVHELCLKLI

SEQ ID NO: 16 - protein, 1195 aa, Beutenbergia cavernae
MTNPPPPWTLDGGRLRAVVADGGAIARLATDDLELVQHAGSALEPGLLGLWVRDARHGLHPLLGPGSGSRVAVSGAGSQAPAAEAQESSRGGAENLSRMEAAGEWDDGGLAWRVRFSLVPGELAWAWDVELWPTDGEPHDVDVVHAHDVALAPAGMLRANELYVSQYLDVTPLEHPTHGWALAVRQNLPVPGGHPWLVLACTDRAAAYATDAIDVHGLAARAAEPPPLAGGLPSRRRQHEHTLAALQTQQHRLDPDGAWRVSFAALLRTDHPAATGEADLAAVEEAIALARGVRGAAGEAPTAPPRPAVASVYAPARVVPVRDLRTAEVAERWGVPERGVETADDGALLAFAPDAERHVVLGAKERAVLRPHGTILRGGHGLAPDPGTVAVTAWMTGSPLSYLTRGHASSARALTTVRGYLGLHRAYGVRVLVEDVTDGGWHLLDVASAFEMTPDGARWVYVTDGVTIELATSLTSDGAVLDVTSSVPRRFLLACHLATSGTDNAPAPGELETRVTPDGASVVFGPATPLGSVAPGARLTLVATENPQAPSSSREASDNVSGTVGAGDDGTLFDDGASRGLPVVTFASGPTTSLRLDARLHTSREVRDPSARGSWPDEETLVAAERGAEAGSSSADAPPAPEPEPAAVVLPSLALPSTADAAAAADVDAVALAVPWLVRDALVHYLAPRGLEQYTGGAWGTRDVSQGPVELLLALDRPDDVRALLRRVFAAQNADGSWPQAFGFLPGDEDFRHEPPHGDVVFWPVLALGRHLVTTGDAGVLDDVVGYHAGARAEESVLEHALRALDAARAATLPGTHLAAYGHGDWNDSLQPAEPGMTSTLTSSWTVTLHHHALQMLTEGLAGAGVHEELADSLRAEAAAVAADARRHLLVDGELAGYAQLAPPEPAGDDGAAATPAHVTRLLVHPRDDETGLTHSALPMIHAIAEDFFTPDEACHHVQILREHLLGTDGMRLFDRPAQYSGGPMVHFQRAESATFVGREIGLMYVHAHLRWCEALARLGDADGLWLALRQVLGGAVAGAVPGARPRQTNTYFSSSDAAVADRPEFAARYGQVRSGEVAVEGGWRIYSSGPGITVRILAEVLLGIRRRGAWIEVDPVLPPELDGLEARVPLLGGELAVTYRVGTRGAGPSEIRLGGRTIEFERLANPYREGGARVDLEPLRDAVTTHETLEIVLP

SEQ ID NO: 17 - protein, 824 aa, Paneibacillus riograndensis
MMKQPGPASPAGLSGSKEHKKGVVKTAGGAAVVREQQLDQYFTFRDELKEVEFKRHDMPTPWMNYLSNGTFHTMLSHAGGGVAFYKSPQIWRITRYRFFHLPTDRSGPYIYLQDAGTGRYWCPTYEPAFQKPQEWSSSHGLGYTRFEAQEDEVAAKTVYFVGPYENSLIWNLTLTNNSPAVKVLNVYAYAEFGMMEFMRELQWQCYNKHQVSVQYHPKEALVYKYGVENQPKPEETPLVYMMSDTPLHSYDGDREEFIGSYRSESNPAAIEQGGCTGSTLLGGDPCGALQVQVTLQPGETRTVNFFLGTAMDEVEVEQAIEHSREADFVARSYAALKENWDSYLGAWNCELPDADAQRMLNIWNPYQAQRNFLFSRNISFYATGTFRGVGFRDTAQDILAVAPFDPEAAKEKVRLLLGQQYQDGHVNHYFFPNEGWDPVTSIHSDDHLWTALAVWDILAETGDAGFLQESLPFYDGGEGTLYEHLKRAIDFTASRLGPNGFPLMLRSDWNDQLFRVCRQGKGESIWTAMQLGTVLLRMKDLAAASGHPEHAAGYEAMYENQKDLVNTLGWDGQWFRRAVMDDGRFLGTDEHDEAKIWLNAQTWATLSGMAEPDKAVQAMDSVRDILDTELGIKKLHPSITTFPDPADPLTNYNKGTGENGAVFCHANTWAIIAECMLGRGDQAYKYYRQLIPNVAMEQAGIWRYKAEPYVYASNLFGPESDKFGLANVSWLTGTAAWMYIAATQYIMGIKPVLEGLSIDPCIPSEWEGFTVNRRFRGCLYEISVTNSSHNCKGVKEIRVDGQRLEGNIVPAYPEKAMVKVEVIL

SEQ ID NO: 18 - protein, 828 aa, Thermosipho melanesiensis
MEITKRKKEQKYRTSKKNIKENFIEHLNLSLGKDFSNATLWDKYHALALLVKDRVVEKWLKTQKKYYETTDVKRVYYLSIEFLIGRLLYNNLLNLQIDEEVKKAMKDLNLSLEEIAEAEPDAGLGNGGLGRLAACFLDSIATLGLPGYGYGIRYDYGIFKQVIKDGFQIELPDDWLKNGNPWEIERKDRTVKVKFFGRSESYKDEDGNIRFKWVDTFDVLAVPYDTPIIGYGNNVANTLRLWSAKALNEFDFNNFQKGNYIKSAESNAIASAISKVLYPNDAFYSGRELRLKQEYFFVSASLQDIIRRFKKQFGNQFDLFPKRNVIQLNDTHPALAIPELMRILVDEEHLPWEKAWDITIKTFAYTNHTVMPEALEKWEVHLFERLLPRHLEILYEINARFLDNAKKIFNGNIEKIRNVSIFEEGNVKQVRMANLSIVGSFSINGVSKLHTEILKKNVFKDFYEIWPEKFNNKTNGITQRRWLLQCNPGLSKIINETIGSEWITNLDYLKNLEKFADDKSFLDKFYEVKQNNKIRLSRYIEKELDIKVDPESIFDVQVKRLHEYKRQLLNVMHIIYIYQRLKENPNLDIYPRTFIFGAKAAPGYRMAKLIIKLINSVANVVNNDKDIKDKIKVIFIPNYNVSLAEIIIPAANVSEQISTAGKEASGTGNMKFALNGALTIGTLDGANIEIKECVGDENIFIFGLTAEQVSKLKESRLYNPYDIYLRNEHIRNILDAINNSFFEKNNPDLFKDIFQSLLFGINGSQADEYMLLADFDSYKTRHKEIDLTYRNKYTWNKKALLNVARVGIFSSDRTIREYAEDIWKVKKI

SEQ ID NO: 19 - protein, 861 aa, Thermosynechococcus sp.
MNNPDTPNGYPVHSVSHTHPTANDEHCDLYIESDRTGMTVQTLKRAFVDNLHYIQGKDAMFATPYDYFMALAYTVRDRLLHRRIKTAQTYFEQDAKVVYYLSAEFLIGRLLLNNLINVGLYEQTKQAMADFGLDLNELMEREPEPGLGNGGLGRLAACFLDSLATLEIPAVGYGIRYEFGIFEQIITNGWQHEVPDNWLRFGNPWEIARPDYNVEVKFGGHTEAYTDAQGHYRVRWIPSTTVFGTPYDTPIPGYGKNTVNTLRLWSARAAQDFNLQVFNAGDYTQAVSEKTFSENISKVLYPNDNTPQGKELRLRQQYFFVACSLQDIIRLYLRRHTSFDAFPDKVAIQLNDTHPAIAVAELMRLLVDEYQLTWEKAWHITQRTFAYTNHTLLAEALERWSVDLFGQLLPRHLEIIYEINYRFLNEVRLRYPGNTARLARMSLIEEGYPKQVRMAHLACVGSHTVNGVAELHTELIKQELMRDFYEMYPNKFQNKTNGITPRRWLLMSNPRLASLITETLQSDRWITHLDDLRGLEPYATDPAFQAKWQQIKQANKERLAEYIWRNNQIEVDPYSLFDIQIKRIHEYKRQHLAVLHIITLYEQIKANPSIDIQPRTFIFGGKAAPGYFMAKMIIKLINSVADMVNHDSDVNGRLKVVFLSNYSVSLGEMVYPAADLSEQISTAGKEASGTGNMKFALNGALTIGTLDGANVEIRQEVGAENFFLFGLTAQEVMSLKAEGYNPREYYNSNPMLKKVIDSLISNYFNPREPGLFEPIVSSLLNEDPYMLLADYQSYVDCQQRAAQAFRNKSHWTQMSILNVARMGKFSSDRTIAEYCKEIWHVEPVPVSLDTCRPAFRPSRIS

SEQ ID NO: 20 - protein, 44 aa, synthetic construct
MHHHHHHSSGLVPRGSGMKETAAAKFERQHMDSPDLGTDDDDKA

(19) United States
(12) Patent Application Publication
(10) Pub. No.: US 2026/0078420 A1
(43) Pub. Date: Mar. 19, 2026

(54) COMPOSITIONS AND METHODS FOR THE PRODUCTION OF 1,2-BETA-OLIGOGLUCANS

(71) Applicant: Cargill, Incorporated, Wayzata, MN (US)

(72) Inventors: Ronny Leontina Marcel Vercauteren, Haasdonk (BE); Tom Desmet, Nevele (BE); Zorica Ubiparip, Drongen (BE)

(73) Assignee: Cargill, Incorporated, Wayzata, MN (US)

(21) Appl. No.: 19/106,274

(22) PCT Filed: Aug. 30, 2023
(86) PCT No.: PCT/US2023/073127

§ 371 Date: Feb. 25, 2025

(30) Foreign Application Priority Data
Aug. 30, 2022 (EP) 22192796.5
Dec. 13, 2022 (EP) 22213127.8

(51) Int. Cl. C12P 19/04; C08B 37/00; C12N 9/10; C12P 19/18

(52) U.S. Cl. CPC C12P 19/04; C08B 37/0024; C12N 9/1051; C12P 19/18; C12Y 204/01097

(57) ABSTRACT

Disclosed herein are compositions and methods for the productions of β-1,2-oligoglucans. Said compositions include glucose-1-phospahte, a prime molecule, and a β-glucan phosphorylase with β-1,2-glucan phosphorylase activity. For example, the βGP with β-1,2-glucan phosphorylase activity may have a sequence at least 70% at least 75%, at least 80%, at least 85%, at least 90%, at least 95%, at least 99%, or 100% identical to at least one of SEQ ID NOs:10, 11, 13, 15, or 16.

### CROSS-REFERENCE TO RELATED APPLICATIONS

This application claims the benefit of EP Application Serial No. 22192796.5, filed Aug. 30, 2022 and EP 22213127.8, filed Dec. 13, 2022, Application which is incorporated by reference herein in its entirety.

### REFERENCE TO A SEQUENCE LISTING SUBMITTED ELECTRONICALLY

The content of the Sequence Listing XML file of the sequence listing named “PT-1130-WO-PCT.xml” which is 35,438 bytes in size created on Aug. 29, 2023 and electronically submitted herewith the application is incorporated by reference in its entirety.

### BACKGROUND

In nature, β-glucans are produced by bacteria and play an important role in the invasion and immunomodulation of infected mammalian or plant cells. Commercially, most β-glucans are produced from cell wall extracts of yeasts, fungi, and plants, although some limited fermentation based synthetic methods exist. The purified β-glucans are typically obtained by acidic hydrolysis steps, followed by selective precipitation using organic solvents. The extraction process and biological origin of β-glucans lead to significant variations in their physicochemical and functional properties, including their branching pattern, molecular weight distribution, viscosity, and concentration in the biological matrix. While some glycosyltransferases are specialized for the synthesis of β-glucans, the high cost of their nucleotide-activated donor sugars is a serious limitation for their commercial exploitation. Therefore, a need in the art exists for more consistent, reproducible, and commercially relevant compositions and methods for the production of β-glucans, and specifically β-1,2-oligoglucans.

### SUMMARY

The present disclosure provides a method for the production of 1,2-beta-oligoglucans, the method comprising contacting α-D-glucose-1-phosphate (G1P) with a beta (β)-glucan-phosphorylase (βGP) to produce 1,2-beta-oligoglucans. The method may additionally comprise the step of contacting a substrate with an alpha(α)-glucan-phosphorylase (αGP) in the presence of inorganic phosphate (e.g., sodium phosphate or potassium phosphate) to produce the glucose-1-phosphate. The substrate may have a degree of polymerization (DP) equal to or greater than 4. The substrate may be selected from the group consisting of maltodextrin, starch liquefact, trehalose, sucrose, cellulose, cellodextrins, cellobiose, and combinations thereof. The βGP GIP contacting step may be carried out at a pH between 6.0 and 7.5 and/or the substrate αGP contacting step is carried out at a pH between 6.5 and 8.0.

The disclosure also provides a composition comprising i) a beta(β)-glucan-phosphorylase (βGP); ii) α-D-glucose-1-phosphate (G1P); and iii) a primer molecule. The composition may additionally comprise a 1,2-beta-oligoglucan, a phosphatase inhibitor (e.g., sodium molybdate), a buffer, and/or a reducing agent. The primer molecule may be selected from the group consisting of D-glucose, sophorose, laminaribiose, cellobiose, gentiobiose, and combinations thereof.

The disclosure also provides a composition comprising an alpha(α)-glucan-phosphorylase (αGP), inorganic phosphate, and a substrate (e.g., maltodextrin, starch, starch liquefact, trehalose, sucrose, cellulose, cellodextrins, cellobiose, and combinations thereof).

In the compositions and methods described herein, the αGP may be a glycoside hydrolase 94 enzyme. The αGP may have an amino acid sequence at least 80%, at least 85%, at least 90%, or at least 95% identical to at least one of SEQ ID NOs: 1, 2, 3, 4, 5, or 18, preferably a sequence at least 80%, at least 85%, at least 95%, or at least 95% identical to at least one of SEQ ID NOs: 1, 2, or 5, or most preferably a sequence at least 90% identical to SEQ ID NO:5.

In the compositions and methods described herein, the βGP may be a glycosyl transferase 35 enzyme. The βGP may have an amino acid sequence at least 80%, at least 85%, at least 90%, or at least 95% identical to at least one of SEQ ID NOs: 10, 11, 13, 15, and 16, preferably a sequence at least 90% identical to SEQ ID NO:11.

The disclosure also provides for the use of the αGP and/or βGP compositions described herein to produce the 1,2-beta-oligoglucan compositions described herein.

Also provided herein is a 1,2-beta-olioglucan composition produced by the method of any one of claims 1-5 and 10-11, wherein the composition has a polydispersity between 2 and 40, a degree of polymerization (DP) of about 6-150, and a viscosity between 800 and 1200 mPas at 50° C. In general, the 1,2-beta-olioglucan composition is not digestible.

The disclosure further provides a vector comprising a nucleic acid encoding a polypeptide at least 80%, at least 85%, at least 90%, or at least 95% identical to SEQ ID NO:5. The disclosure also provides a cell comprising said vector.

### BRIEF DESCRIPTION OF THE FIGURES

This patent or application contains at least one drawing executed in color. Copies of this patent or patent application publication with color drawings will be provided by the Office upon request and the payment of the necessary fee.

The drawings illustrate generally, by way of example, but not by way of limitation, various aspects discussed in the present document.

FIG. 1 shows an SDS-PAGE gel of A1, A3, A5, A6, and A8 expression products from a 100 mL overnight culture of E. coli BL21 (DE3) cells, grown at either 20° C. or 30° C. and induced chemically with 1 mM IPTG.

FIG. 2 shows an SDS-PAGE gel of αGP enzymes after heat treatment of 1 hr at 60° C. For each enzyme, soluble and insoluble fractions are depicted in the left and right columns, respectively.

FIG. 3 shows crude cell extract on 12% SDS-PAGE gel before (left) and after (right) HIS-tag purification. Arrows are pointing towards the position of the 3 thermostable αGP candidates.

FIG. 4 shows specific activity of αGP candidate enzymes as a function of temperature.

FIG. 5 shows specific activity of αGP candidate enzymes retained after incubation at 55° C. for the specified time period. Standard deviation is ≤7% (A1), ≤14% (A3), and ≤20% (A8).

FIG. 6 shows specific activity of candidate αGP enzymes on two different maltodextrin substrates.

FIG. 7 shows a Michaelis-Menten graph for the A1 αGP.

FIG. 8 shows a Michaelis-Menten graph for the A3 αGP.

FIG. 9 shows a Michaelis-Menten graph for the A8 αGP.

FIG. 10 shows Michaelis-Menten graphs for the A8 αGP enzyme at a maximum maltodextrin concentration of 24% (left) and 60% (right).

FIG. 11 shows an SDS-PAGE gel of βGP expression products from a 100 mL overnight culture (B1, B2, B3, B4, B5, B6, and B8) or 250 mL overnight culture (B7, B11, B12, B13, and B14) of E. coli BL21 (DE3) cells, grown at 20° C., and chemically induced with 1 mM IPTG. “a” indicates the soluble crude extract fraction and “b” indicates the insoluble cruse extract fraction. Full arrows show soluble protein bands and dashed arrows show insoluble protein bands.

FIG. 12 shows SDS-PAGE gel of βGP enzymes after heat treatment of 1 hr at 60° C. For each enzyme, soluble (“hS”) and insoluble (“hI”) fractions are depicted in the left and right columns respectively.

FIG. 13 shows release of phosphate (Pi) and glucose (Glc) over time as the result of the assays described in Example 4.

FIG. 14 shows a thin-layer chromatography chromatogram of the reaction products of the B6 assays outline in Example 5 and Table 10.

FIG. 15 shows an image of the anion-exchange chromatography results for B6 activity on sophorose. Individual components in the reaction mixture are included (three lower lines). Formation of glucans of different DP was demonstrated by a “row of peaks” clearly not belonging to any of the individual components in the assay mixture.

FIG. 16 shows graphs representing the relationships between temperature or pH and activity for the B6 enzyme.

FIG. 17 shows graphs representing the thermostability of the B6 after incubation at 50° C. Standard deviation is ≤17%.

FIG. 18 shows a Michaelis-Menten graph of B6 activity on a sophorose substrate.

FIG. 19 shows HIS-tag purification of βGP enzymes B7, B11, B12, B13, and B14.

FIG. 20 shows TLC plates with reaction mixtures loaded for reactions of the B7, B11, B12, B13, and B14 enzymes on each of glucose, sophorose, laminaribiose, gentiobiose, and cellobiose. Arrows are pointing towards products formed. Reactions with no activity have two spots in the column corresponding to the GIP and the substrate.

FIG. 21 shows an anion exchange chromatogram showing the activity of the B7 enzyme on a sophorose substrate. Text refers to know reaction mixture components (e.g., sophorose and G1P) and reaction products (e.g., formed oligo- and polysaccharides).

FIG. 22 shows an anion exchange chromatogram showing the activity of the B12 enzyme on a glucose substrate. Text refers to know peaks (e.g., glucose, gentiobiose/cellobiose, sophorose/laminaribiose, and G1P) and reaction products (e.g., formed disaccharides: β-1,2 linked sophorose and/or β-1,3 linked laminaribiose).

FIG. 23 shows an anion exchange chromatogram showing the activity of the B12 enzyme on a glucose substrate to form laminaribiose.

FIG. 24 shows an anion exchange chromatogram showing the activity of the B13 enzyme on a laminaribiose substrate. Text refers to known peaks (e.g., glucose, laminaribiose, and G1P) and to reaction products (e.g., sugars formed).

FIG. 25 shows an anion exchange chromatogram showing the activity of the B13 enzyme on a sophorose substrate. Text refers to known peaks (e.g., glucose, sophorose) and to reaction products (formed oligosaccharides).

FIG. 26 shows an anion exchanges chromatogram showing the activity of the B13 enzyme on glucose. Text refers to known peaks (e.g., glucose and G1P) and reaction products (e.g., sugars formed).

FIG. 27 shows an anion exchange chromatogram showing the activity of the B6 enzyme on a combination substrate syrup as outlined in Example 8.

FIG. 28 shows a chromatograph obtained by HPLC fingerprint of debranched Zulkowsky starch maltodextrin.

FIG. 29 shows a chromatogram obtained by HLPC fingerprint of debranched soluble starch maltodextrin.

FIG. 30 shows a chromatogram obtained by HPLC fingerprint of debranched DE 4-7 maltodextrin.

FIG. 31 shows a chromatogram obtained by HPLC fingerprint of debranched DE 13-17 maltodextrin.

FIG. 32 shows a chromatogram obtained by HPLC fingerprint of debranched maltodextrin 01910.

FIG. 33 shows a chromatogram obtained by HPLC fingerprint of debranched maltodextrin 01912.

FIG. 34 shows a graph of the conversion of debranched soluble starch to GIP using the A8 αGP enzyme.

FIG. 35 shows a graph of the conversion of debranched Zulkowsky starch to GIP using the A8 αGP enzyme.

FIG. 36 shows a graph of the conversion of debranched MDX DE 4-7 to GIP using the A8 αGP enzyme.

FIG. 37 shows a graph of the conversion of debranched MDX DE 13-17 to GIP using the A8 αGP enzyme.

FIG. 38 shows pictures of β-glucans synthesized starting from 0.5, 1, or 1.5M GIP and using the B6 βGP crude extract released by sonication (left) or homogenization (right).

FIG. 39 shows a graph of the effect of temperature and Sumizyme GOP on β-glucan synthesis.

FIG. 40 shows a graph of the effect of Sumizyme GOP and/or sodium molybdate on β-glucan synthesis.

FIG. 41 shows a 1H-NMR spectra of the β-1,2-glucan produced using the B6 βGP enzyme.

FIG. 42 shows a 13C-NMR spectra of the β-1,2-glucan produced using the B6 βGP enzyme.

FIG. 43 shows a sample of 120 g β-1,2-glucan (tops) and chromatograms of oligosaccharide (middle) and high molecular weight GPC analysis of the β-1,2-glucan.

FIG. 44 shows a graph of shear stress versus shear rate for β-1,2-glucan samples in the temperature range of 20-80° C.

FIG. 45 shows a graph of viscosity versus shear rate for β-1,2-glucan samples in the temperature range of 20-80° C.

FIG. 46 show the 20-30° C. Newtonian viscosity of β-1,2-glucan compared with glucose (top), sucrose (middle), and standard maltodextrins (bottom).

FIG. 47 shows glucose release from isomaltulose, sucromalt, promitor 70, and β-1,2-glucan in an in vitro digestibility assay.

FIG. 48 shows B-glucan synthesis starting from 0.5M G1P and pure B6 with glucose as primer.

FIG. 49 shows B-glucan synthesis starting from 0.2M GIP and pure B6 with glucose as primer.

FIG. 50 shows graphs of the molecular weight distribution of β-glucans produced by a reaction of 0.2M (top) or (0.5M) GIP with pure B6 after a 72-hour reaction time.

FIG. 51 shows the reaction scheme used in Example 12.

FIG. 52 shows β-glucan synthesis under the recited conditions, as outlined in Example 12.

FIG. 53 shows viscosity measurements for the recited starch and/or β-glucan compositions, as outlined in Example 13.

FIG. 54 shows viscosity measurements for the recited starch and/or β-glucan compositions as outlined in Example 13.

FIG. 55 shows a phylogenic tree of αGPs.

FIGS. 56A-56D show SDS-PAGE gel images of preserved protein band of stable (A) ArGP, TtGP and TaGP and (C) weak protein band of TsGP and strong protein band of TmGP, after 1 h of incubation at 60° C. In FIGS. 56A and 56C “c” indicates the soluble fraction and “d” indicates the insoluble fraction after heat treatment. FIGS. 56B and 56D show AtGP, TIGP, TaGP, TsGP and TmGP purified by affinity (His6-tag) chromatography. Arrows indicate the protein band.

FIG. 57 shows the effect of temperature on TmGP and TsGP activity, in comparison to TaGP. The temperature profiles were determined using 50 mM phosphate buffer and 2% maltodextrin mixture as substrates at pH 7. Relative activity is calculated by the percentage of the values against the maximum.

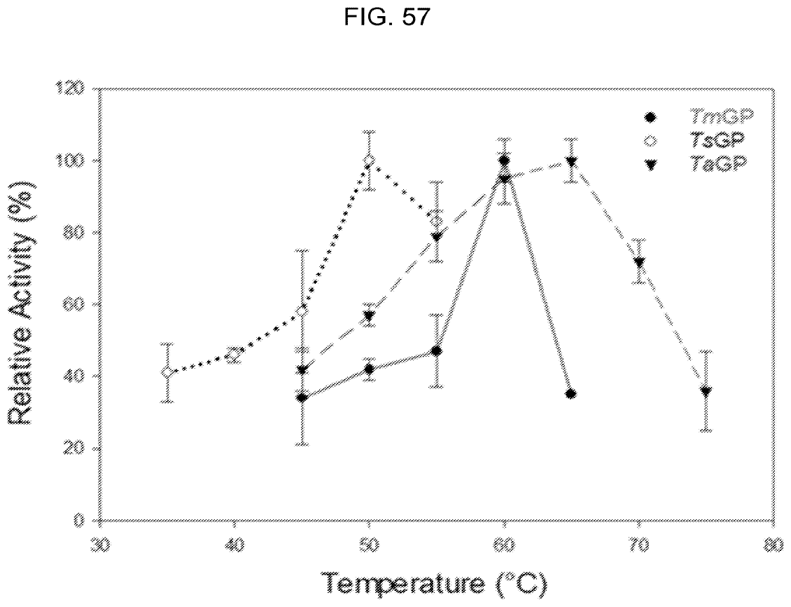

FIG. 58 shows a 1H-NMR spectra of the β-1,2-glucan produced using the B7 βGP enzyme. Boxed sections of NMR spectra are enlarged in FIG. 59.

FIG. 59 shows an enlarged view of the boxed sections of the NMR spectra of FIG. 58.

FIG. 60 shows a 13C-NMR spectra of the β-1,2-glucan produced using the B7 βGP enzyme. Boxed sections of NMR spectra are enlarged in FIG. 61.

FIG. 61 shows an enlarged view of the boxed sections of the NMR spectra of FIG. 60.

FIG. 62 shows a 1H-NMR spectra of the β-1,2-glucan produced using the B13 βGP enzyme. Boxed sections of NMR spectra are enlarged in FIG. 63.

FIG. 63 shows an enlarged view of the boxed sections of the NMR spectra of FIG. 62

FIG. 64 shows a C13-NMR spectra of the β-1,2-glucan produced using the B13 βGP enzyme.

FIG. 65 shows an enlarged view of the boxed sections of the NMR spectra of FIG. 64.

### DETAILED DESCRIPTION

Reference will now be made in detail to certain aspects of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

In this document, the terms “a,” “an,” or “the” are used to include one or more than one unless the context clearly dictates otherwise. The term “or” is used to refer to a nonexclusive “or” unless otherwise indicated. All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

Unless expressly stated, ppm (parts per million), percentage, and ratios are on a by weight basis. Percentage on a by weight basis is also referred to as wt % or % (wt) below.

Unless defined otherwise, all technical and scientific terms used herein have the same meaning as commonly understood by one skilled in the art to which this invention belongs. As used herein, each of the following terms has the meaning associated with it as defined below.

This disclosure relates to β-1,2-oligoglucan compositions and compositions and methods for making said β-1,2-oligoglucans. In general, compositions for making β-1,2-oligoglucans include α-D-glucose-1-phosphate (GIP, also referred to as “alpha-D-glucose-1-phosphate”), a primer molecule, and a β-glucan phosphorylase (βGP) enzyme. Methods for making β-1,2-oligoglucans described herein include incubating the compositions including GIP, the primer molecule, and the βGP at a temperature and for a time sufficient to produce the β-1,2-oligoglucan. The disclosure also provides compositions for the synthesis of GIP from maltodextrin the compositions including a maltodextrin, phosphate, and an a-glucan phosphorylase (αGP). Methods for making GIP described herein include incubating the compositions including maltodextrin, phosphate, and the αGP at a temperature and for a time sufficient to produce the G1P. The disclosure further provides compositions comprising the produced β-1,2-oligoglucans.

As used herein, the terms “polypeptide” and “peptide” are used interchangeably and refer to the collective primary, secondary, tertiary, and quaternary amino acid sequence and structure necessary to give the recited macromolecule its function and properties. As used herein, “enzyme” or “biosynthetic pathway enzyme” refer to a protein that catalyzes a chemical reaction. The recitation of any particular enzyme, either independently or as part of a biosynthetic pathway is understood to include the co-factors, co-enzymes, and metals necessary for the enzyme to properly function. A summary of the amino acids and their three and one letter symbols as understood in the art is presented in Table 1. The amino acid name, three letter symbol, and one letter symbol are used interchangeably herein.

TABLE 1
Amino Acid three and one letter symbols

| Amino Acid | Three letter symbol | One letter symbol |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Variants or sequences having substantial identity or homology with the polypeptides described herein can be utilized in the practice of the disclosed pigments, compositions, and methods. Such sequences can be referred to as variants or modified sequences. That is, a polypeptide sequence can be modified yet still retain the ability to exhibit the desired activity. Generally, the variant or modified sequence may include or greater than about 45%, 45%, 50%, 55%, 60%, 65%, 70%, 75%, 80%, 85%, 90%, or 95% sequence identity with the wild-type, naturally occurring polypeptide sequence, or with a variant polypeptide as described herein.

As used herein, the phrases “% sequence identity,” “% identity,” and “percent identity,” are used interchangeably and refer to the percentage of residue matches between at least two amino acid sequences or at least two nucleic acid sequences aligned using a standardized algorithm. Methods of amino acid and nucleic acid sequence alignment are well-known. Sequence alignment and generation of sequence identity include global alignments and local alignments which are carried out using computational approaches. An alignment can be performed using BLAST (National Center for Biological Information (NCBI) Basic Local Alignment Search Tool) version 2.2.31 software with default parameters. Amino acid % sequence identity between amino acid sequences can be determined using standard protein BLAST with the following default parameters: Max target sequences: 100; Short queries: Automatically adjust parameters for short input sequences; Expect threshold: 10; Word size: 6; Max matches in a query range: 0; Matrix: BLOSUM62; Gap Costs: (Existence: 11, Extension: 1); Compositional adjustments: Conditional compositional score matrix adjustment; Filter: none selected; Mask: none selected. Nucleic acid % sequence identity between nucleic acid sequences can be determined using standard nucleotide BLAST with the following default parameters: Max target sequences: 100; Short queries: Automatically adjust parameters for short input sequences; Expect threshold: 10; Word size: 28; Max matches in a query range: 0; Match/Mismatch Scores: 1, −2; Gap costs: Linear; Filter: Low complexity regions; Mask: Mask for lookup table only. A sequence having an identity score of XX % (for example, 80%) with regard to a reference sequence using the NCBI BLAST version 2.2.31 algorithm with default parameters is considered to be at least XX % identical or, equivalently, have XX % sequence identity to the reference sequence.

Polypeptide or polynucleotide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The polypeptides disclosed herein may include “variant” polypeptides, “mutants,” and “derivatives thereof.” As used herein the term “wild-type” is a term of the art understood by skilled persons and means the typical form of a polypeptide as it occurs in nature as distinguished from variant or mutant forms. As used herein, a “variant, “mutant,” or “derivative” refers to a polypeptide molecule having an amino acid sequence that differs from a reference protein or polypeptide molecule. A variant or mutant may have one or more insertions, deletions, or substitutions of an amino acid residue relative to a reference molecule.

The amino acid sequences of the polypeptide variants, mutants, derivatives, or fragments as contemplated herein may include conservative amino acid substitutions relative to a reference amino acid sequence. For example, a variant, mutant, derivative, or fragment polypeptide may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. Conservative amino acid substitutions generally maintain (a) the structure of the polypeptide backbone in the area of the substitution, for example, as a beta sheet or alpha helical conformation, (b) the charge and/or hydrophobicity of the molecule at the site of the substitution, and/or (c) the bulk of the side chain.

Compositions for the synthesis of β-1,2-oligoglucans include GIP, a primer molecule, and a β-glucan phosphorylase as described herein. The compositions may additionally include a buffer and/or a reducing agent. Suitable buffers may include, but are not limited to, a phosphate buffer (e.g., a potassium phosphate buffer, a sodium phosphate buffer, or preferably a citric acid Na₂HPO₄ buffer system pH 5.5-7.6). Suitable reducing agents may include, but are not limited to, dithiothreitol (DTT), tris(2-carboxyethyl) phosphine (TCEP), ascorbic acid, cysteine, sodium bisulfite, SO₂, and combinations thereof. The composition may have a pH between 6.5 and 7.5.

As used herein, “GIP” and “glucose-1-phosphate” are used interchangeable and refer to a glucose molecule with a phosphate group on the 1′ carbon. The GIP may be present in the compositions and methods described herein at a concentration between 0.05M and 2.0M, between 0.1M and 1.75M, or between 0.2M and 1.5M.

As used herein, “βGP,” “beta-GP,” “beta-glucan-phosphorylase,” and “β-glucan-phosphorylase” are used interchangeably and refer to an enzyme that reversibly catalyzes the phosphorylation of glycosidic linkages in β-glucans to form glucose-1-phosphate. The reverse reaction catalyzes the synthesis of β-glucans through phosphorolytic transfer of glycose from glucose-1-phosphate to an acceptor primer molecule (e.g., glucose, sophorose, laminaribiose, cellobiose, and the like). The βGP enzyme may be regioselective, for example, selectively catalyzing linkages at the β-1,2 position of glycose in the primer molecule. As used herein, enzyme that catalyze regioselective addition of glucose at the β-1,2 position of the primer molecule from GIP are said to have “β-1,2-glucan phosphorylase activity.” The βGP may be annotated in the Carbohydrate-Active enZYmes database (CAZY) as belonging to glycoside hydrolase family 94 (GH94). The βGP with β-1,2-glucan phosphorylase activity may also be referred to in the art as a laminaribiose phosphorylase. The βGP with β-1,2-glucan phosphorylase activity may have a sequence at least 70%, at least 75%, at least 80%, at least 85%, at least 90%, at least 95%, at least 99%, or 100% identical to at least one of SEQ ID NOs: 10, 11, 13, 15, or 16.

The βGP polypeptide with β-1,2-glucan phosphorylase activity may be, or may be derived from, the Paenibacillus sp. laminaribiose phosphorylase (PsLBP) of SEQ ID NO:10. The βGP polypeptide with β-1,2-glucan phosphorylase activity may have an amino acid sequence with at least 70%, at least 75%, at least 80%, at least 85%, at least 90%, at least 95%, at least 99%, or 100% sequence identity to the amino acid sequence of SEQ ID NO:10.

The βGP polypeptide with β-1,2-glucan phosphorylase activity may be, or may be derived from, the Rhizobium tropici β-1,2-glucan phosphorylase (RtSOGP) of SEQ ID NO:11. The βGP polypeptide with β-1,2-glucan phosphorylase activity may have an amino acid sequence with at least 70%, at least 75%, at least 80%, at least 85%, at least 90%, at least 95%, at least 99%, or 100% sequence identity to the amino acid sequence of SEQ ID NO:11.

The βGP polypeptide with β-1,2-glucan phosphorylase activity may be, or may be derived from, the Clostridium saccharoperbutylaceonicum β-1,2-glucan phosphorylase (CsSOGP) of SEQ ID NO:13. The βGP polypeptide with β-1,2-glucan phosphorylase activity may have an amino acid sequence with at least 70%, at least 75%, at least 80%, at least 85%, at least 90%, at least 95%, at least 99%, or 100% sequence identity to the amino acid sequence of SEQ ID NO:13.

The βGP polypeptide with β-1,2-glucan phosphorylase activity may be, or may be derived from, the Paenibacillus stellifer β-glucan phosphorylase (PsGP) of SEQ ID NO:15. The βGP polypeptide with β-1,2-glucan phosphorylase activity may have an amino acid sequence with at least 70%, at least 75%, at least 80%, at least 85%, at least 90%, at least 95%, at least 99%, or 100% sequence identity to the amino acid sequence of SEQ ID NO:15.

The βGP polypeptide with β-1,2-glucan phosphorylase activity may be, or may be derived from, the Beutenbergia cavernae β-1,2-glucan phosphorylase (BcSOGP) of SEQ ID NO: 16. The βGP polypeptide with β-1,2-glucan phosphorylase activity may have an amino acid sequence with at least 70%, at least 75%, at least 80%, at least 85%, at least 90%, at least 95%, at least 99%, or 100% sequence identity to the amino acid sequence of SEQ ID NO:16.

As used herein, “primer molecule” refers to a mono-, di-, or polysaccharide containing D-glucose. The primer molecular may be, but is not limited to, D-glucose, sophorose, laminaribiose, cellobiose, gentiobiose, and combinations thereof. Suitable primers are known and described in the art. See, for example, Ubiparip, et al. (“β-glucan phosphorylases in carbohydrate synthesis,” Applied Microbiology and Biotechnology, 2021, 105:4073-4087) Without wishing to be bound by any particular theory or mode of action, it is believed that the primer molecule acts as an initial scaffold upon with additional glucose molecules are added to produce the β-1,2-oligoglucan. If the βGP enzyme is provided to the composition as a crude cell extract or lysate, the primer molecule may be residual glucose in the extract/lysate and no additional primer molecule is required. The primer molecule may also be referred to in the art as an acceptor molecule. In the compositions and methods described herein, the primer molecule may be at a concentration of at least 1 mM, 2 mM, 5 mM, 10 mM, 20 mM, 50 mM, 100 mM, 150 mM, 200 mM, 250 mM, or at least 300 mM. In the compositions and methods described herein, the primer molecule may be present at a concentration of at most 5 mM, 10 mM, 20 mM, 50 mM, 100 mM, 150 mM, or at most 200 mM. Without being bound to any particular theory or mode of action, the concentrations of the primer molecule and the GIP may be tailored to produce a β-1,2-oligoglucan of a particular degree of polymerization. For example, as demonstrated in Example 11 below, the combination of a low concentration of the primer molecule may be used to generate a β-1,2-oligoglucan with a high degree of polymerization.

The compositions for the synthesis of β-1,2-oligoglucans may optionally include a phosphatase inhibitor. Suitable phosphatase inhibitors are known and described in the art. For example, the phosphatase inhibitor may be sodium molybdate (Na₂MoO₄), (NH₄)₆Mo₇O₂₄, ATP, Cu²⁺ (e.g., CuSO₄), GDP, GTP, HgCl₂, iodoacetic acid, Na₃AsO₄, Na₃VO₄, NaF, sodium citrate, tartaric acid, or a combination thereof. A phosphatase inhibitor such as sodium molybdate may be added to the compositions for the synthesis of β-1,2-oligoglucans at a concentration between 1-500 mM, between 50-400 mM, or between 100-300 mM.

The compositions for the synthesis of β-1,2-oligoglucans may be used in a method for making β-1,2-oligoglucans, as described herein. The method includes incubating a composition including glucose-1-phosphate (GIP), a primer molecule, and a βGP with β-1,2-glucan phosphorylase activity for a time and under conditions sufficient to produce β-1,2-oligoglucans. Based on the disclosure herein, a skilled artisan will understand the time and conditions suitable to produce the β-1,2-oligoglucans. The composition may be incubated at a temperature between 30° C. and 70° C., between 35° C. and 65° C., between 37° C. and 60° C., between 40° C. and 57° C., or between 40° C. and 50° C. The composition may be incubated at a temperature of about 35° C., about 37° C., about 40° C., about 45° C., about 50° C., about 55° C., about 57° C., about 60° C., about 65° C. The composition may have a pH between 5.5 and 7.5. The composition may have a pH of about 5.5, about 6.0, about 6.5, about 7.0, or about 7.5. The composition may be incubated for at least 30 minutes, at least 1 hour, at least 5 hours, at least 10 hours, at least 12 hours, at least 15 hours, at least 18 hours, at least 20 hours, at least 24 hours, at least 30 hours, at least 35 hours, at least 40 hours, or at least 44 hours. The composition may be incubated with shaking or agitation.

The disclosure also provides compositions for the synthesis of GIP from maltodextrin. Compositions for the synthesis of GIP include a glucan substrate, inorganic phosphate, and an α-glucan phosphorylase as described herein. The compositions may additionally include a buffer and/or a reducing agent. Suitable buffers may include, but are not limited to, a phosphate buffer (e.g., K₂HPO₄/KH₂PO₄ or Na₂HPO₄/NaH₂PO₄). Suitable reducing agents may include, but are not limited to, dithiothreitol (DTT), tris(2-carboxyethyl) phosphine (TCEP), ascorbic acid, cysteine, sodium bisulfite, SO₂, and combinations thereof. The composition may have a pH between 6.5 and 8.

The glucan substrate may be a maltodextrin, a starch (e.g., starch liquefact), glycogen, amylose, amylopectin, trehalose, sucrose, laminaribiose, cellulose, cellodextrins, cellobiose, or combinations thereof. Preferably, the glucan substrate is a maltodextrin. The glucan substrate may be present in the composition for the synthesis of GIP at a concentration between 100-2000 mM, 150-1800 mM, or 200-1000 mM. The glucan substrate concentration may also be based on the dry substances percentage of the composition. For example, the maltodextrin may be added such that the composition has a dry substances percentage between 1%-30%, between 2% and 25%, or between 5% and 20%. The glucan substrate may be added such that the composition has a dry substances percentage of about 1, 2, 3, 4, 5, 6, 7, 8, 9, 10, 11, 12, 13, 14, 15, 16, 17, 18, 19, or about 20%. In general, the glucan substrate for use in the compositions for the synthesis of GIP will have a degree of polymerization (DP) of at least 5. Without wishing to be bound by any particular theory or mode of action, a glucan substrate with a DP of 4 or less cannot be used as a substrate for the αGP enzyme for the synthesis of G1P.

The glucan substrate (e.g., maltodextrin) may be debranched prior to addition to the composition for the synthesis of GIP. Suitable methods for debranching maltodextrin and other glucan substrates are known and described in the art. See, for example, Ling Hii et al. (“Pullulanase: Role in starch hydrolysis and potential industrial applications,” Enzyme Research, 2012, 921362) and Moller et al. (“Structure and function of α-glucan debranching enzymes,” Cell. Mol. Life Sci., 2016, 73:2619-2641). For example, the maltodextrin may be incubated with a pullulanase enzyme (such as the one sold under the tradename OPTIMAX™ L-1000) and/or an iso-amylase enzyme to debranch the maltodextrin.

The inorganic phosphate may be provided in the composition in any suitable form. Suitable forms of inorganic phosphate include, but are not limited to, sodium phosphate (Na₂HPO₄), potassium phosphate (KH₂PO₄), other salts, and combinations thereof. The inorganic phosphate may be present in the composition at a concentration between 100-2000 mM, 150-1800 mM, or 200-1000 mM. The inorganic phosphate may be present in the composition at a starting concentration of about 100, 200, 300, 400, 500, 600, 700, 800, 900, 1000, 1100, 1200, 1300, 1400, 1500, 1600, 1700, 1800, 1900, or about 2000 mM.

The glucan substrate (e.g., maltodextrin) and the inorganic phosphate may be present in the composition for the synthesis of GIP at any suitable ratio. For example, the maltodextrin and inorganic phosphate may be present in the composition at a starting molar ratio of 1:0.25, 1:0.5, or 1:1. The maltodextrin and inorganic phosphate may be present in the composition at a starting molar ratio between 1:0.1 and 1:2, between 1:0.2 and 1:1.5, between 1:0.25 and 1:1.25, or between 1:0.5 and 1:1.

As used herein, “αGP,” “alpha-GP,” “alpha-glucan-phosphorylase,” and “α-glucan-phosphorylase” are used interchangeably and refer to an enzyme that reversibly catalyzes the phosphorolytic cleavage of a α-1,4 glycosidic linkage to form glucose-1-phosphate. See Scheme 1. The α-1,4 glycosidic linkage may be cleavage in substrate such as glycogens, starches, and maltodextrins. The αGP may be annotated in the Carbohydrate-Active enZYmes database (CAZY) as belonging to glycosyltransferase family 35 (GT35). The αGP may have a sequence at least 70%, at least 75%, at least 80%, at least 85%, at least 90%, at least 95%, at least 99%, or 100% identical to at least one of SEQ ID NOs: 1, 2, 3, 4, 5, or 18. The αGP may have a sequence at least 70%, at least 75%, at least 80%, at least 85%, at least 90%, at least 95%, at least 99%, or 100% identical to at least one of SEQ ID NOs: 1, 2, 5, or 18. The αGP may have a sequence at least 70%, at least 75%, at least 80%, at least 85%, at least 90%, at least 95%, at least 99%, or 100% identical to at least one of SEQ ID NOs: 1, 2, or 5.

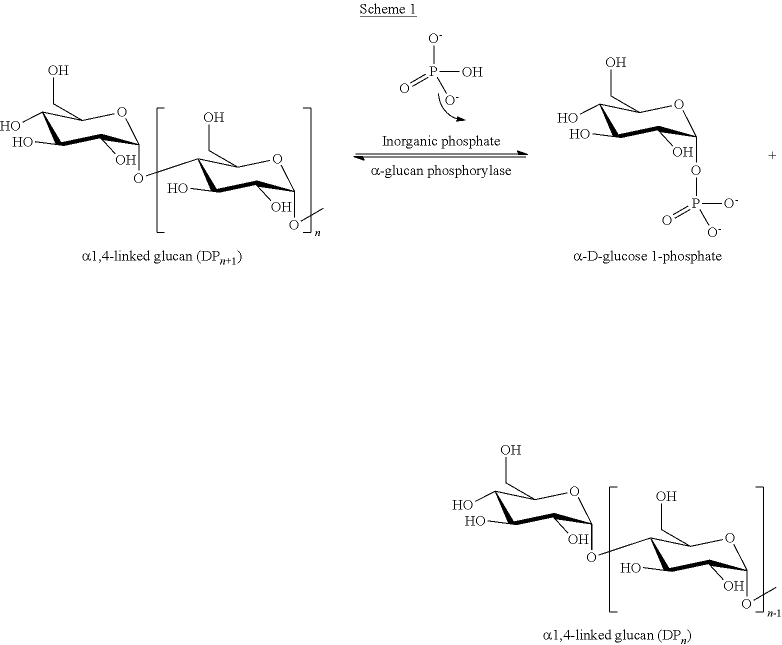

The αGP polypeptide may be, or may be derived from, the Anaerolinea thermophila αGP (A1GP) of SEQ ID NO: 1. The αGP polypeptide may have an amino acid sequence with at least 70%, at least 75%, at least 80%, at least 85%, at least 90%, at least 95%, at least 99%, or 100% sequence identity to the amino acid sequence of SEQ ID NO:1.

The αGP polypeptide may be, or may be derived from, the Thermobaculum terrenum αGP (TtGP) of SEQ ID NO:2. The αGP polypeptide may have an amino acid sequence with at least 70%, at least 75%, at least 80%, at least 85%, at least 90%, at least 95%, at least 99%, or 100% sequence identity to the amino acid sequence of SEQ ID NO:2.

The αGP polypeptide may be, or may be derived from, the Thermincola potens αGP (TpGP) of SEQ ID NO:3. The αGP polypeptide may have an amino acid sequence with at least 70%, at least 75%, at least 80%, at least 85%, at least 90%, at least 95%, at least 99%, or 100% sequence identity to the amino acid sequence of SEQ ID NO:3.

The αGP polypeptide may be, or may be derived from, the Thermodesulfobacterium geofontis αGP (TgGP) of SEQ ID NO:4. The αGP polypeptide may have an amino acid sequence with at least 70%, at least 75%, at least 80%, at least 85%, at least 90%, at least 95%, at least 99%, or 100% sequence identity to the amino acid sequence of SEQ ID NO:4.

The αGP polypeptide may be, or may be derived from, the Thermosipho africanus αGP (TaGp) of SEQ ID NO:5. The αGP polypeptide may have an amino acid sequence with at least 70%, at least 75%, at least 80%, at least 85%, at least 90%, at least 95%, at least 99%, or 100% sequence identity to the amino acid sequence of SEQ ID NO:5.

The αGP polypeptide may be, or may be derived from, the Thermosipho melanesiensis αGP (TmGP) of SEQ ID NO: 18. The αGP polypeptide may have an amino acid sequence with at least 70%, at least 75%, at least 80%, at least 85%, at least 90%, at least 95%, at least 99%, or 100% sequence identity to the amino acid sequence of SEQ ID NO: 18.

The compositions for the synthesis of GIP may be used in a method for making GIP, as described herein. The method includes incubating a composition including a glucan substrate, inorganic phosphate, and an αGP polypeptide for a time and under conditions suitable to produce GIP. Based on the disclosure herein, a skilled artisan will understand the time and conditions suitable to produce GIP from the glucan substrate. The composition may be incubated at a temperature between 30° C. and 70° C., between 35° C. and 65° C., between 37° C. and 60° C., between 40° C. and 57° C., or between 40° C. and 50° C. The composition may be incubated at a temperature of about 35° C., about 37° C., about 40° C., about 45° C., about 50° C., about 55° C., about 57° C., about 60° C., about 65° C. The composition may have a pH between 5.5 and 8.0, between 6.0 and 7.5, or between 7.0 and 7.5. The composition may have a pH of about 5.5, about 6.0, about 6.5, about 7.0, about 7.5, or about 8.0. The composition may be incubated for at least 30 minutes, at least 1 hour, at least 5 hours, at least 10 hours, at least 12 hours, at least 15 hours, at least 18 hours, at least 20 hours, at least 24 hours, at least 30 hours, at least 35 hours, at least 40 hours, or at least 44 hours. The composition may be incubated with shaking or agitation. The method may additionally include a step for debranching the glucan substrate (e.g., maltodextrin) prior to GIP synthesis or simultaneously with GIP synthesis in the same reaction vessel.

In general, the β-1,2-oligoglucans produced by the compositions and methods described herein are characterized by a polydispersity (Mw/Mn) between 2 and 40 (e.g., 4-30, 6-20, 8-15, or any value or subrange therein), a degree of polymerization (DP) of at least 150 (e.g., at least 150, at least 200, at least 250, at least 300, at least 350, at least 400, between 150-400, or between 200 and 350), a molecular weight between 20 kDa and 200 kDa (e.g., between 25 kDa and 150 kDa, between 40 kDa and 125 kDa, or between 50 kDa and 100 kDa), and a viscosity greater than 200 cP (e.g., between 200 and 350 cP or between 250 and 350 cP) when measured at 30° C., 50 rpm agitation, and a concentration of 30% dry substances. Alternatively, the β-1,2-oligoglucans produced by the compositions and methods described herein are characterized by a polydispersity (Mw/Mn) between 2 and 40 (e.g., 4-30, 6-20, 8-15, or any value or subrange therein), a degree of polymerization (DP) of between 3 and 150 (e.g., between 3-150, between 6-100, or between 10-50), a molecular weight between 20 kDa and 200 kDa (e.g., between 25 kDa and 150 kDa, between 40 kDa and 125 kDa, or between 50 kDa and 100 kDa), and a viscosity greater than 200 cP (e.g., between 200 and 350 cP or between 250 and 350 cP) when measured at 30° C., 50 rpm agitation, and a concentration of 30% dry substances.

The β-1,2-oligoglucans produced by the compositions and methods described herein are non-digestible. As used herein, “non-digestible” refers to a composition which, when assessed using the in vitro digestion assay as described in Garcia-Campayo et al., “Digestion of food ingredients and food using an in vitro model integrating intestinal mucosal enzymes,” Food and Nutrition Sciences, 2018, 9:711-734, has a glucose release of less than 1%, less than 5%, less than 7.5% or less than 10%.

The produced β-1,2-oligoglucans may be used in a β-1,2-oligoglucan composition. The β-1,2-oligoglucan composition may be used in the preparation of a food product, a beverage product, and/or an animal feed product. For example, the β-1,2-oligoglucan composition may be used to replace a portion or all of a bulking agent, a fiber, or another low-calorie ingredient used in the preparation of the food, beverage, or animal feed product. The β-1,2-oligoglucan composition may be used as a prebiotic or immune stimulate or may be used in the preparation of a prebiotic or immune stimulate composition. Without wishing to be bound by any particular theory or mode of action, it is believed that due to the non-digestible nature of the β-1,2-oligoglucan produced by the compositions and methods described herein, the β-1,2-oligoglucan may be used to replace other caloric components of food, beverage, and/or animal feed products but retain beneficial bulking agent or fiber properties of said food, beverage, and/or animal feed product.

As used herein, terms “polynucleotide,” “polynucleotide sequence,” and “nucleic acid sequence,” and “nucleic acid,” are used interchangeably and refer to a sequence of nucleotides or any fragment thereof. These phrases also refer to DNA or RNA of natural or synthetic origin, which may be single-stranded or double-stranded and may represent the sense or the antisense strand. The DNA polynucleotides may be a cDNA or a genomic DNA sequence.

A polynucleotide is said to encode a polypeptide if, in its native state or when manipulated by methods known to those skilled in the art, it can be transcribed and/or translated to produce the polypeptide or a fragment thereof. The anti-sense strand of such a polynucleotide is also said to encode the sequence.

Those of skill in the art understand the degeneracy of the genetic code and that a variety of polynucleotides can encode the same polypeptide. In some aspects, the polynucleotides (i.e., polynucleotides encoding an αGP or βGP polypeptide) may be codon-optimized for expression in a particular cell including, without limitation, a plant cell, bacterial cell, fungal cell, or animal cell. While polypeptides encoded by polynucleotide sequences found in various species are disclosed herein any polynucleotide sequences may be used which encodes a desired form of the polypeptides described herein. Thus, non-naturally occurring sequences may be used. These may be desirable, for example, to enhance expression in heterologous expression systems of polypeptides or proteins. Computer programs for generating degenerate coding sequences are available and can be used for this purpose. Pencil, paper, the genetic code, and a human hand can also be used to generate degenerate coding sequences.

Also provided herein are polynucleotides encoding an αGP polypeptide. The polynucleotide may encode any of the αGP polypeptides described herein, for example, the polynucleotide may encode a polypeptide at least 80%, at least 85%, at least 90%, at least 95%, or at least 98% identical to at least one of SEQ ID NOs: 1, 2, 3, 4, 5, and 18. The polynucleotide encoding the αGP polypeptide may be a cDNA sequence encoding a polypeptide at least 80%, at least 85%, at least 90%, at least 95%, or at least 98% identical to at least one of SEQ ID NOs: 1, 2, 3, 4, 5, and 18.

Also provided herein are polynucleotides encoding a βGP polypeptide. The polynucleotide may encode any of the βGP polypeptides described herein, for example, the polynucleotide may encode a polypeptide at least 70%, at least 75%, least 80%, at least 85%, at least 90%, at least 95%, or at least 98% identical to at least one of SEQ ID NOs: 10, 11, 13, 15 and 16. The polynucleotide encoding the βGP polypeptide may be a cDNA sequence encoding a polypeptide at least 80%, at least 85%, at least 90%, at least 95%, or at least 98% identical to at least one of SEQ ID NOs: 1, 2, 3, 4, 5, and 18.

The polypeptides described herein may be provided as part of a construct. As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies. The construct may be a vector including a promoter operably linked to the polynucleotide encoding the thermolabile EforRed polypeptide. As used herein, the term “vector” refers to a polynucleotide capable of transporting another polynucleotide to which it has been linked. The vector may be a plasmid, which refers to a circular double-stranded DNA loop into which additional DNA segments may be integrated.

Cells including any of the polynucleotides, constructs, or vectors described herein are also provided. The cell may be a procaryotic cell or a eukaryotic cell. Suitable procaryotic cells include bacteria cell, for example, Escherichia coli and Bacillus subtilis cells. Suitable eukaryotic cells include, but are not limited to, fungal cells, plant cells, and animal cells. Suitable fungal cells include, but are not limited to, Fusarium venenatum, Pichia pastoris, Saccharomyces cerevisiae, Kluyveromyces lactis, Yarrowia lipolytica, Trichoderma reesei, Issatchenkia orientalis, and Aspergillus niger cells. For example, a cell comprising a polynucleotide encoding at least one of SEQ ID NOs: 1, 2, 3, 4, 5, 10, 11, 13, 15, 16, or 18 may be used to produce an αGP and/or βGP polypeptide for use in the compositions and methods described herein. Suitable methods for cell-based protein expression are known and described in the art and one of skill in the art would understand how to suitably express and purify any of the polypeptides described herein from a cell based or cell free system.

### EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather should be construed to encompass any and all variations which become evident as a result of the teaching provided herein.

### Example 1—Alpha(α)-Glucan-Phosphorylase (αGP) and Beta(β)-Glucan-Phosphorylase (βGP) Selection

Approximately 1600 sequences were extracted from the Carbohydrate-Active enZYmes database (CAZY) for selection of possible αGPs (CAZY annotated glycosyltransferase family 35 members (GT35)) and around 1000 sequences for possible βGPs (CAZY annotated glycoside hydrolase family 94 members (GH94)). Sequence extraction and creation of the lists with unique sequences was done computationally. The lists were used for sequence alignment and construction of protein phylogenic trees in the ClustalOmega EMBL-EBI online tool (Multiple Sequence Alignment). Visualization of the trees was performed in the online tool for display, annotation, and management of phylogenetic trees—iTOL (Interactive Tree of Life).

Selection of candidate sequences focused in part on genes and enzymes from thermophilic sources given that these enzymes should show stability and activity at higher temperatures. Candidate βGPs were selected based on the likely probability that the chosen enzymes will perform the desired activity. Selection was also informed by using previously characterized enzymes sequences. The 7 selected αGPs and the 12 selected βGPs are outlined in Table 2. Protein size was calculated using the ProtParam (ExPASy, SIB Bioinformatics Resource Portal) online tool.

TABLE 2

| | | | GenBank | Calculated | SEQ |
|---|---|---|---|---|---|
| Enzyme | | | Accession | Molecular | ID |
| Reference | Source | Enzyme | Number | weight (kDa) | NO: |
| A1 or | Anaerolinea thermophila | αGP | BAJ62538.1 | 89.67 | 1 |
| AtGP | | | | | |
| A3 or | Thermobaculum terrenum | αGP | ACZ41795.1 | 83.51 | 2 |
| TtGP | | | | | |
| A6 or | Thermincola potens | αGP | ADG81774.1 | 98.55 | 3 |
| TpGP | | | | | |
| A5 or | Thermodesulfobacterium | αGP | AEH22393.1 | 105.47 | 4 |
| TgGp | geofontis | | | | |
| A8 TaGP | Thermosipho africanus | αGP | ACJ74578.1 | 101.57 | 5 |
| TmGP | Thermosipho melanesiensis | αGP | WP_012057991.1 | | 18 |
| TsGP | Thermosynechococcus sp. | αGP | AHB89325.1 | | 19 |
| B1 or | Spirochaeta africana | βGP | AFG36802.1 | 110.10128 | 6 |
| SaGP | | | | | |
| B2 or | Clostridium saccharolyticum | βGP | ADL04208.1 | 118.64428 | 7 |
| CsGP | | | | | |
| B3 or | Halorhabdus tiamatea | βGP | CCQ33042.1 | 108.19688 | 8 |
| HtGP | | | | | |
| B4 or | Halothermotrix orenii | βGP | ACL69175.1 | 108.98968 | 9 |
| HoGP | | | | | |
| B5 or | Paenibacillus sp. | βGP | AIQ34676.1 | 106.95888 | 10 |
| PsLBP | | | | | |
| B6 or | Rhizobium tropici | βGP | AGB75122.1 | 123.64068 | 11 |
| RtSOGP | | | | | |
| B8 or | Listeria monocytogenes | βGP | CDG45646.1 | 128.05998 | 12 |
| LmGP | | | | | |
| B7 or | Clostridium | βGP | AGF59367.1 | 129.96 | 13 |
| CsSOGP | saccharoperbutylacetonicum | | | | |
| B11 or | Acholeplasma brassicae | βGP | CCV65810.1 | 95.43 | 14 |
| AbGP | | | | | |
| B12 or | Paenibacillus stellifer | βGP | AIQ65632.1 | 101.86 | 15 |
| PsLBP | | | | | |
| B13 or | Beutenbergia cavernae | βGP | ACQ81818.1 | 125.73 | 16 |
| BcSOGP | | | | | |
| B14 or | Paneibacillus riograndensis | βGP | CQR58309.1 | 92.98 | 17 |
| PrGP | | | | | |

### Example 2—αGP Expression, Stability, and Activity

Synthetic genes encoding the αGP enzymes recited in Table 2 were codon optimized for expression in E. coli and subcloned into a pET30a(+) plasmid vector with a 6-HIS-Tag and linker (SEQ ID NO:20) on the N terminus. E. coli BL21 (DE3) cells were transformed with the pET30a(+) vector and the cells were grown under conditions in which expression of the enzymes was chemically induced.

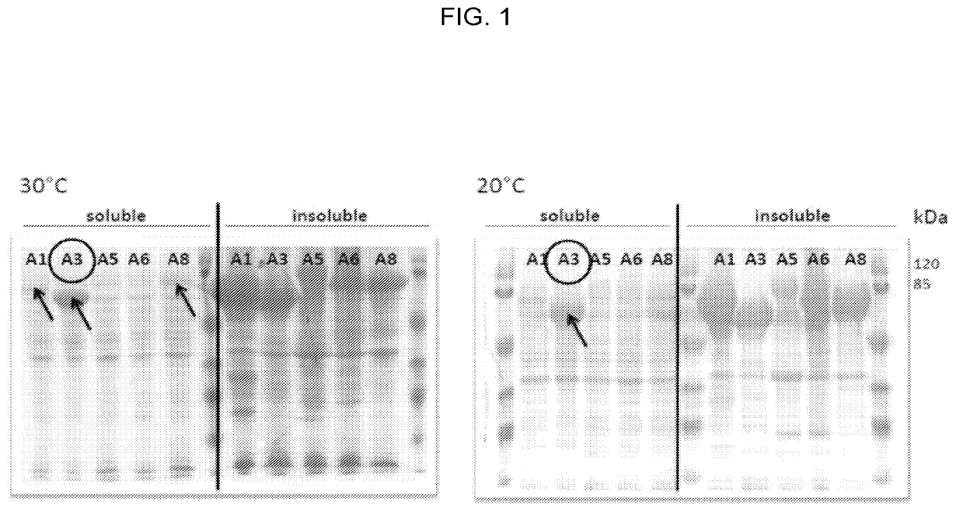

The SDS-PAGE in FIG. 1 shows that 3 out of the 5 enzymes candidates expressed very well in the soluble fraction (A1, A3, and A8) with A3 being the most abundant. Similar results were seen for expression done at both 20° C. and 30° C. While A5 and A6 showed no clear band visible on the gel, low expression variants may still be active, and theses enzymes were tested further.

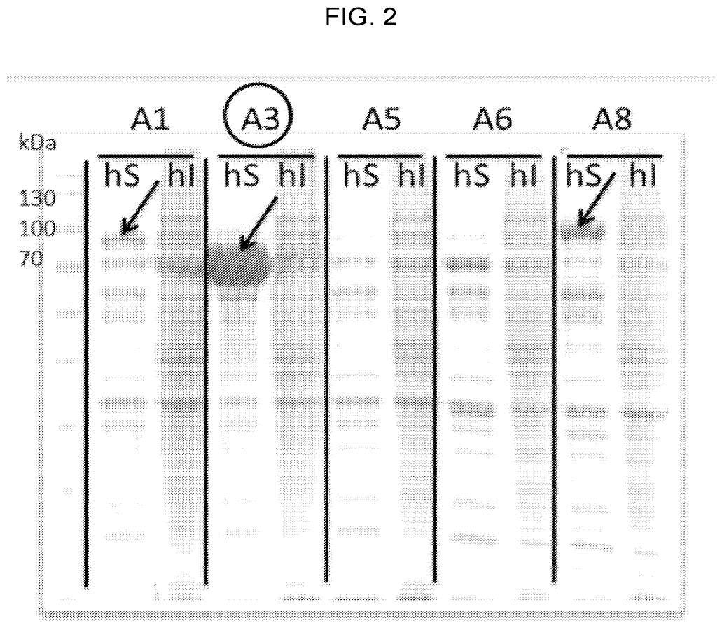

Initial stability tests were performed by incubating crude protein extracts for 1 hour at 60° C. After incubation, the extract was centrifuged to pellet down denatured proteins. Soluble and insoluble fraction (said insoluble fraction containing denatured proteins) were examined by SDS-PAGE (FIG. 2). The results demonstrated that A1, A3, and A8 enzymes remain present in the soluble fraction at the correct molecular weight, and therefore are stable at 60° C.

Activity of αGP enzymes was analyzed using a glucose-1-phosphate enzyme assay. Two types of maltodextrins were used as substrates, differing in molecular weight (Mw) and polydispersity (Mw/Mn) (Table 3). MD1 was a maltodextrin substrate with a polydispersity of 23.2 and MD2 was a maltodextrin substrate with a polydispersity of 16.3. Initial results demonstrate that all selected enzymes showed activity at 37° C. and 57° C. on both maltodextrin substrates. Activity was higher for all proteins in assays run at 57° C. (Table 4)

TABLE 3

| | Maltodextrin | Maltodextrin |
|---|---|---|
| Substrate | 1 (MDX1) | 2 (MDX2) |
| Weight average molecular weight (Mw) | 32768 | 18404 |
| Number average molecular weight (Mn) | 1413 | 1129 |
| Polydispersity | 23.2 | 16.3 |

TABLE 4

| | | A1 | A3 | A5 | A6 | A8 |
|---|---|---|---|---|---|---|
| | Incubation | Expressed | Expressed | Expressed | Expressed | Expressed |
| Substrate | Temp. | at 30° C. | at 30° C. | at 30° C. | at 30° C. | at 30° C. |
| MD1 | 37° C. | + | + | + | + | ++ |
| MD2 | 37° C. | + | + | + | + | ++ |
| MD1 | 57° C. | +++ | +++ | +++ | +++ | +++ |
| MD2 | 57° C. | +++ | +++ | +++ | +++ | +++ |

### Example 3—αGP Enzyme Characterization

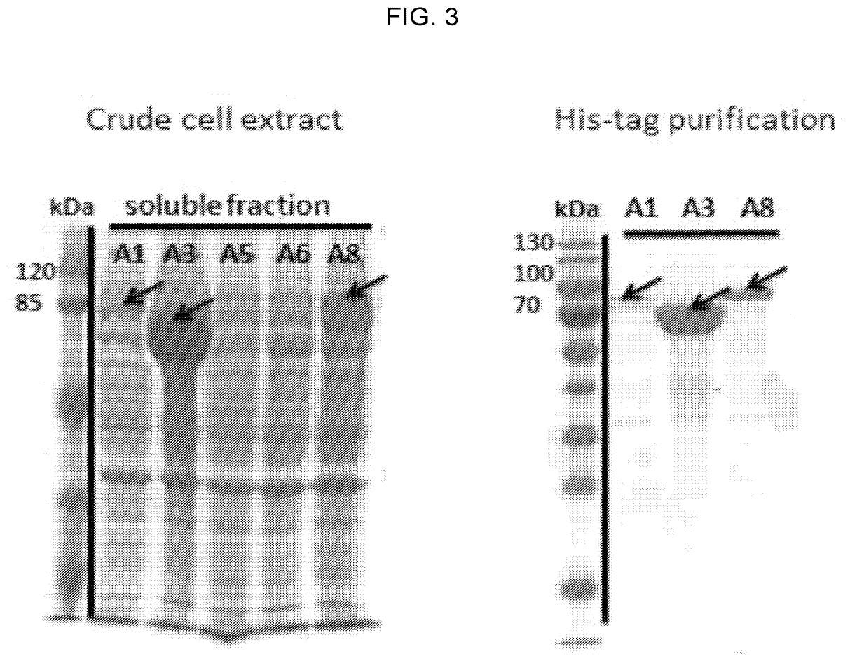

Thermostable enzyme candidates A1, A3, and A8 were further characterized to determine specific activity, residue activity after prolonged incubation at high temperature, pH effects on activity, and temperature effects on activity. FIG. 3 shows crude soluble fraction enzyme extracts as well as the results of His-tag purification of the A1, A3, and A8 enzyme candidates. Approximate enzyme concentrations from the His-tag purification are shown in Table 5.

TABLE 5

| Enzyme | Approximate concentration (mg/L) |
|---|---|
| A1 | 7.5 |
| A3 | 48 |
| A8 | 11.25 |

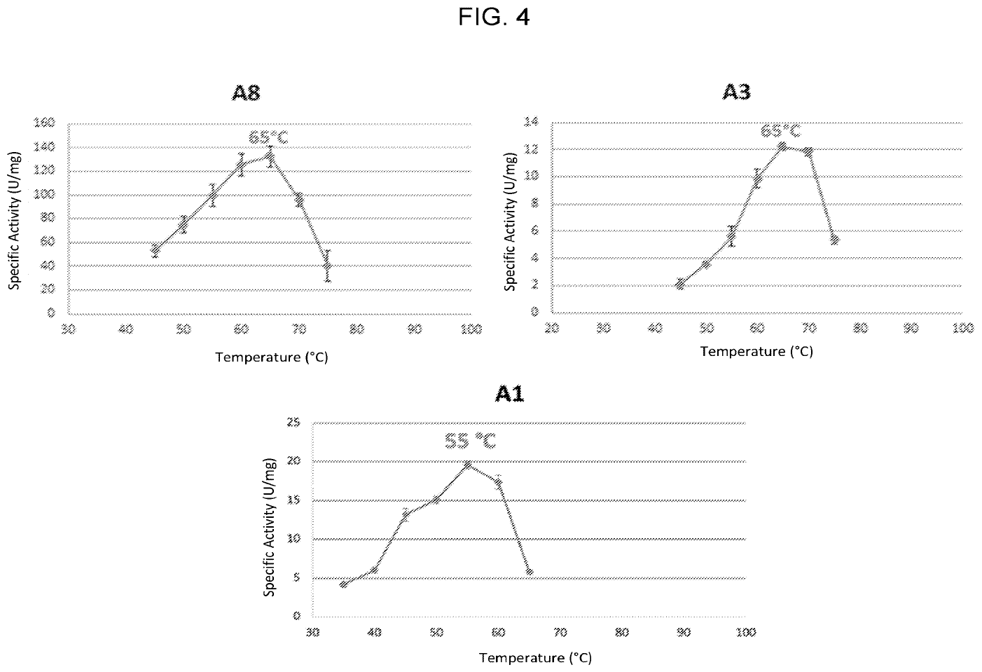

Purified enzymes were further examined to determine specific activity (U/mg), temperature range, pH range, and residual activity after prolonged incubation at 55° C. The results showed that all three enzymes have the highest activity between 55° C. and 65° C. (FIG. 4). Temperature and pH of highest activity, and activity at 55° C. and 65° C. for each enzyme are reported in Table 6. Assays reported in Table 6 were all run with a 2% maltodextrin substrate in 50 mM phosphate buffer at pH 7, unless otherwise indicated.

TABLE 6

| Enzyme Characteristic | A1 | A3 | A8 |
|---|---|---|---|
| Temperature of highest activity (FIG. 4) | 55° C. | 65° C. | 65° C. |
| pH of highest activity | 7 | 7.5 | 7 |
| Activity at 55° C. (U/mg) | 19.6 ± 0.5 | 5.6 ± 0.7 | 99.7 ± 9.1 |
| Activity at 65° C. (U/mg) | 5.8 ± 0.3 | 12.2 ± 0.3 | 132.5 ± 8.4 |

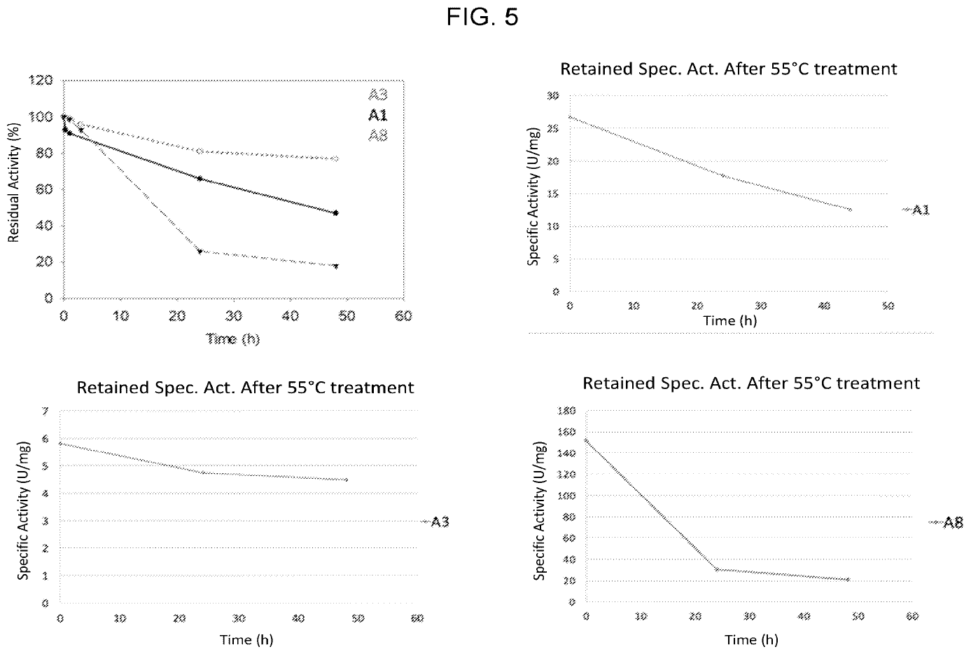

Residual specific activity after prolonged incubation at 55° C. was determined for all αGP candidates. FIG. 5 shows specific activity of an enzyme after incubation at 55° C. for 0, 24, or 44 hours. Time is the enzyme incubation not the assay run time. A3, A1, and A8 maintain roughly 80%, 40%, and 20% of the specific activity after 44 hours of incubation, respectively, Nonetheless, the A8 candidate enzyme still has a higher specific activity at all time points. The A8 enzyme decreased in specific activity about 70% after 24 h at 55° C. and about 80% after 44 h of incubation at 55° C.

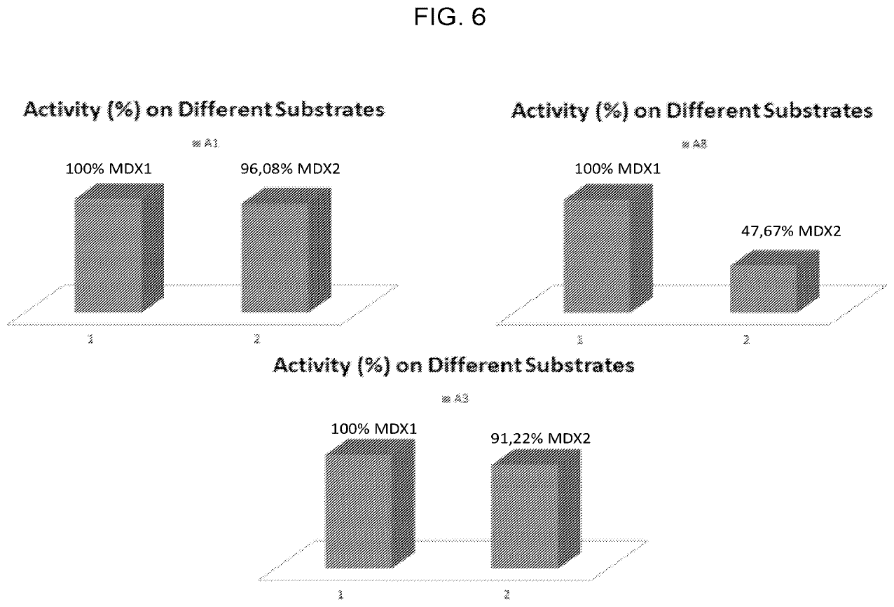

Substrate specificity was also tested for the A1, A3, and A8 candidate enzymes using two different maltodextrin substrates. (FIG. 6) Properties of the two different substrates are outlined in Table 3. The A1 and A3 candidate enzymes had similar activity on both maltodextrin substrates, while A8 has approximately 60% lower initial activity on the MDX2 substrate. (FIG. 6).

### Example 4—αGP Enzyme Kinetic Parameters

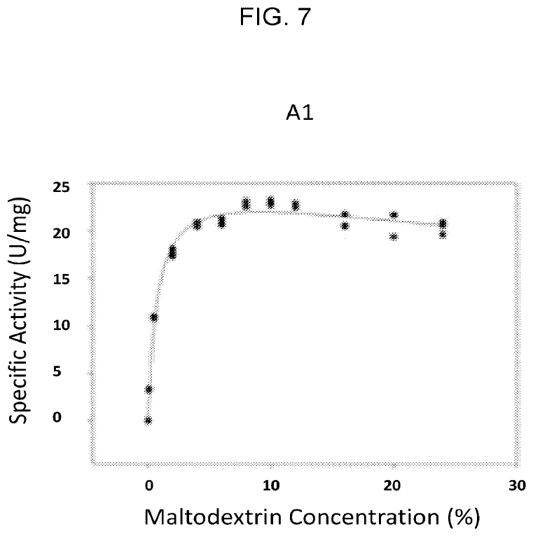

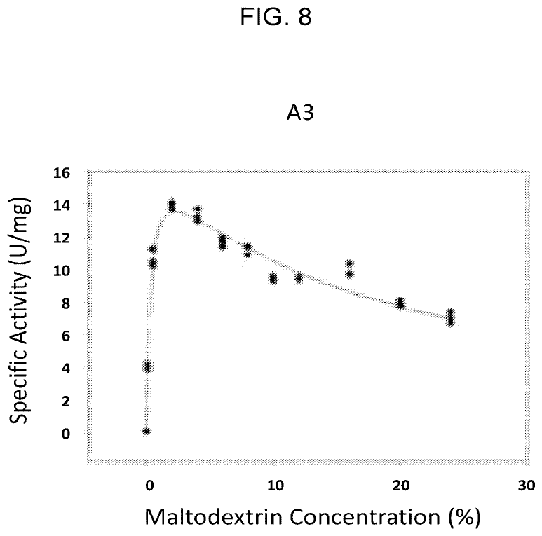

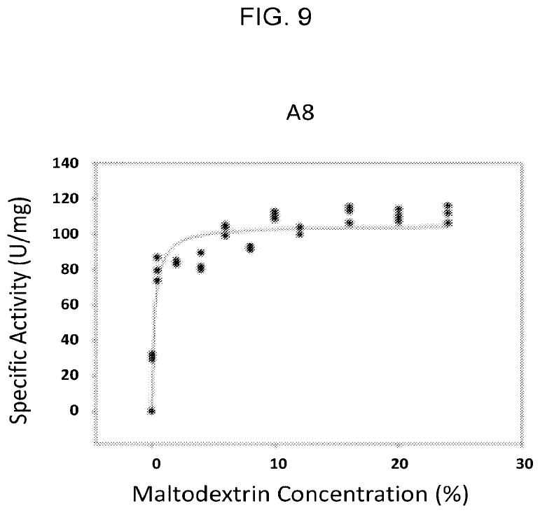

αGP enzyme candidates A1, A3, and A8 were additionally characterized with respect to kinetic parameters. See Table 7 and FIGS. 7-9. Enzyme A8 was not inhibited by the substrate (maltodextrins), while the inhibitor constant was highest for A1. Similarly, A8 has the best kinetic properties, compared to A1 and A3, with the lowest Michaelis-Menten constant (Kₘ), highest turnover number (K_{cat}), and highest K_{cat}/Kₘ ratio.

TABLE 7

| Enzyme | A1 | A3 | A8 |
|---|---|---|---|
| Kₘ (mM) | 0.2 | 0.09 | 0.07 |
| Vₘₐₓ (U mg⁻¹) | 25 | 17 | 105 |
| K_{cat} (s⁻¹) | 38 | 24 | 178 |
| K_{cat}/Kₘ (s⁻¹ mM⁻¹) | 171 | 259 | 2519 |
| Kᵢ (mM) | 35 | 5 | — |

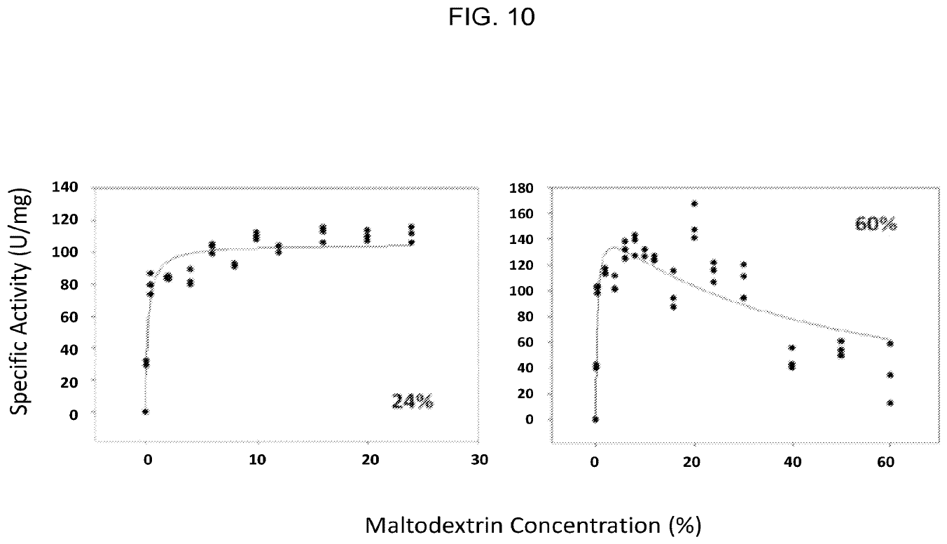

Enzyme A8 was further tested at maltodextrin substrate concentrations up to 60%. See FIG. 10. These results demonstrate that the A8 enzyme is inhibited by the substrate at higher concentrations, likely beginning about 25-30% maltodextrin. Assay in which the maximum maltodextrin concentration was 24% did not show significant substrate inhibition. However, when the maximum maltodextrin concentration was increased to 60%, substrate inhibition is clear beginning at concentrations around approximately 30%. Because the 60% graph in FIG. 10 takes into account all substrate concentrations, the specific activity appears to decline sooner than the 25% graph. However, its more likely that the specific activity remains fairly constant until a substrate concentration around 30% beyond which it sharply declines. Kinetic properties of the assays shown in FIG. 10 are reported in Table 8.

TABLE 8

| Maltodextrin Concentration | | |
|---|---|---|
| Maximum | 24% | 60% |
| Kₘ | 0.17% maltodextrin | 0.33% maltodextrin |
| Vₘₐₓ (U/mg) | 125 | 158 |
| Kᵢ | — | 40% maltodextrin |

### Example 5—βGP Expression, Stability, and Activity

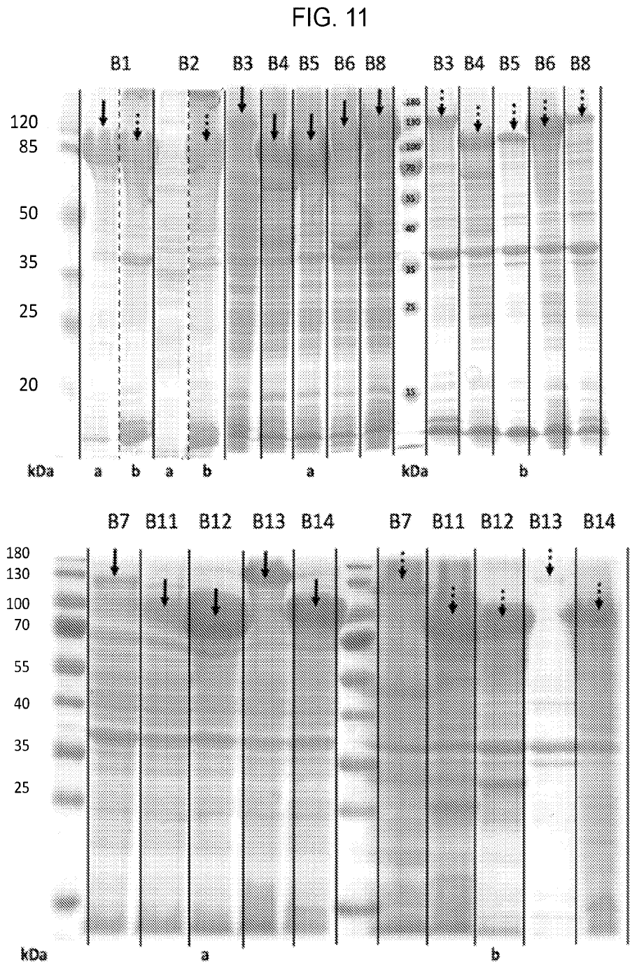

Synthetic genes encoding the βGP enzymes recited in Table 2 were codon optimized for expression in E. coli and subcloned into a pET30a(+) plasmid vector with a 6-HIS-Tag on the N terminus. E. coli BL21 (DE3) cells were transformed with the pET30a(+) vector and the cells were grown under conditions in which expression of the enzymes was chemically induced. The SDS-PAGE in FIG. 11 shows that, apart from B2, all enzyme candidates expressed in the soluble fraction.

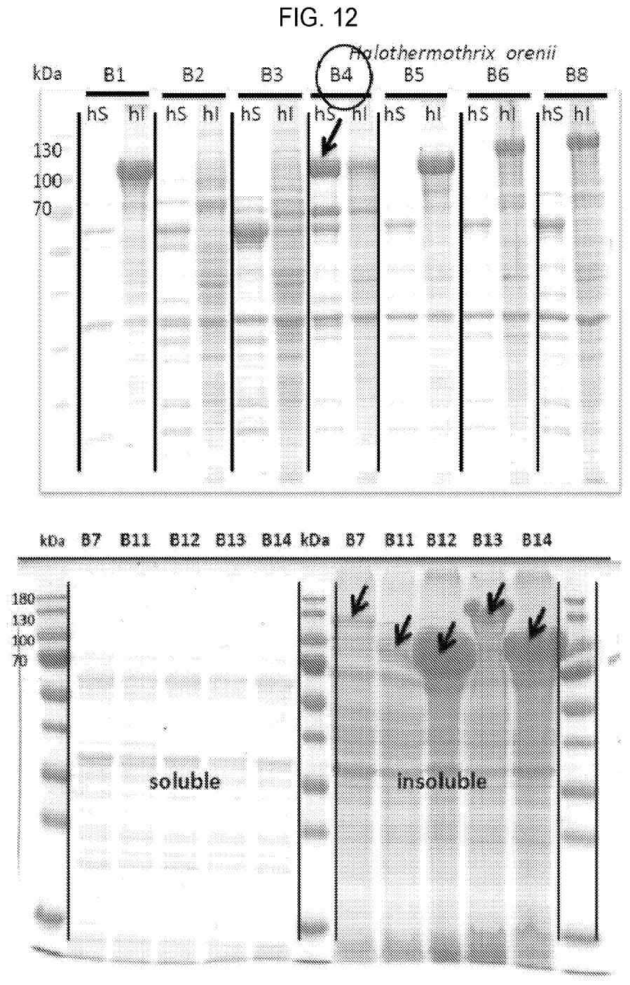

Initial stability tests were performed by incubating crude protein extracts for 1 hour at 60° C. After incubation, the extract was centrifuged to pellet down denatured proteins. Soluble and insoluble fraction (said insoluble fraction containing denatured proteins) were examined by SDS-PAGE (FIG. 12). The results demonstrated that B4 enzyme remains present in the soluble fraction at the correct molecular weight, and therefore is stable at 60° C.

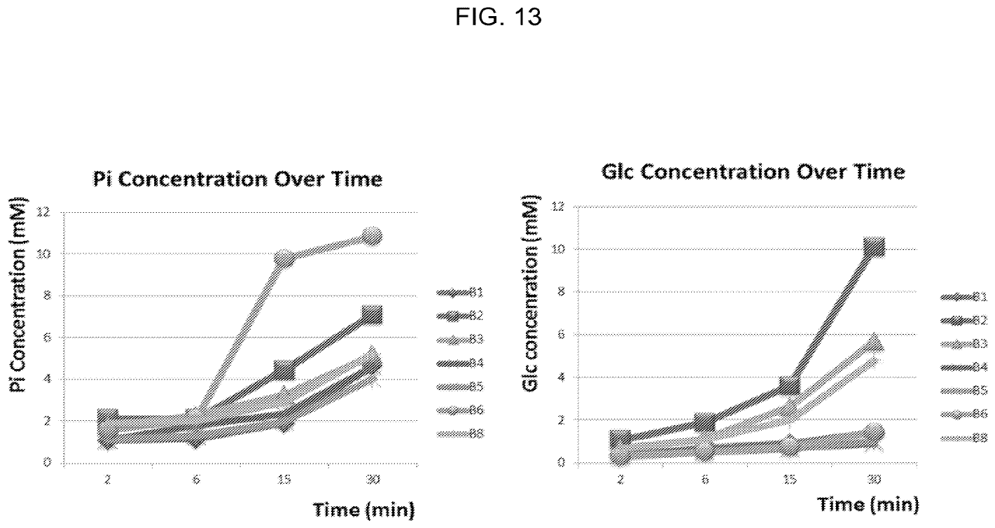

Activity of the βGP enzymes was measured using a combination of the Gawronski phosphate release assay (which measure phosphate concentration, see Gawronski et al., “Microtiter assay for glutamine synthetase biosynthetic activity using inorganic phosphate detection,” Analytical Biochem. 2004 Apr. 1, 327 (1): 114-8) and the GOD-POD assay (which measures glucose concentration). Absence of βGP activity would result in equal concentrations of phosphate and glucose released over time, while higher concentrations of phosphate relative to glucose would indicate the candidate enzyme has the desired βGP activity. The endpoint measurements for both assays were done at 2, 6, 15, and 30 minutes of incubation at 37° C., using 1 mM laminaribiose as a substrate. The results (FIG. 13) showed significantly higher concentrations of phosphate release compare to glucose in 4 out of 7 candidate enzymes (B1, B4, B5, and B6) indicating those enzymes may have the desired activity. The B6 candidate showed the highest difference in glucose and phosphate release over time.

Activity of the B4 βGP enzyme was tested on three different substrates (laminaribiose, sophorose, and cellobiose) using a combination of the Gawronski phosphate release assay (which measure phosphate concentration) and the GOD-POD assay (which measures glucose concentration), as described above. The assays were run at 55° C. using a heat-treated crude cell extract (60° C. for 1 hour). Specific activity (U/mg) for each of the three substrates is reported in Table 9. The different substrates may also be referred to as primers and are the acceptor molecule for the transfer of glucose from GIP. In other words, they are the primer of the resulting oligo- or polysaccharide made using the glucose molecules from GIP.

TABLE 9

Specific Activity (U/mg)

| | Laminaribiose | Sophorose | Cellobiose |
|---|---|---|---|
| Pi release | 0.0317 | 0.0250 | 0.0256 |
| Glc release | 0.0019 | 0.0052 | 0.0088 |
| Pi/Glc ratio | 16.7 | 4.8 | 2.9 |

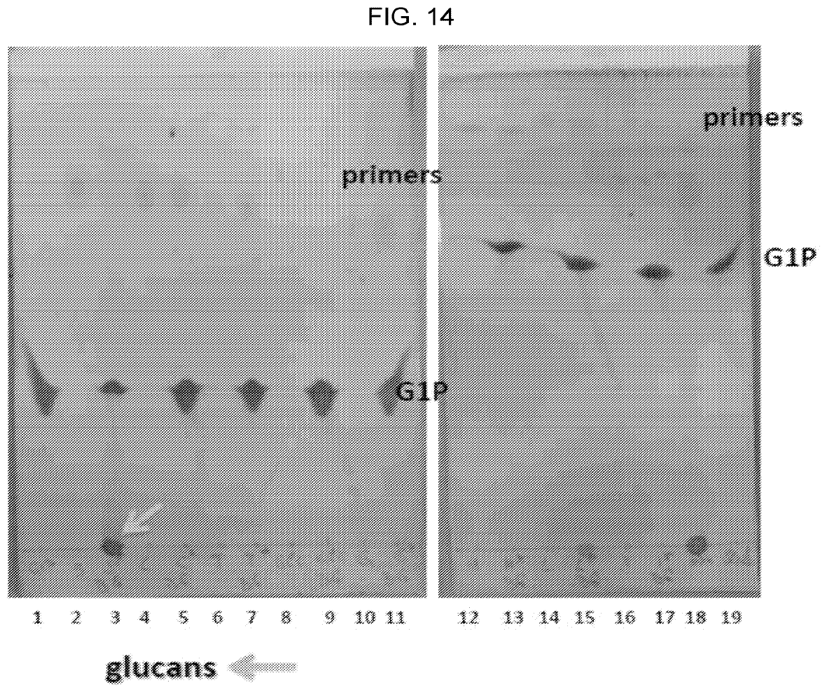

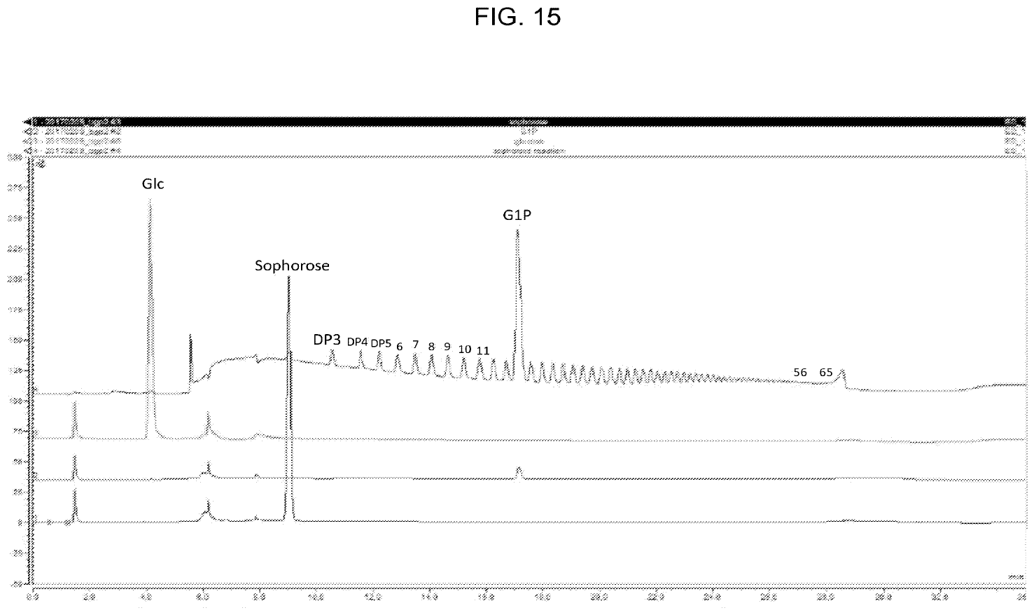

In addition to the phosphate and glucose release assays, the activity of the B6 enzyme was evaluated by product detection methods using thin layer chromatography (TLC). Experiments were done by incubating the enzyme/primer-substrate/GIP mixture at 35° C. and 45° C. for 20 or 40 minutes, after which a small amount was loaded on TLC and a chromatogram was developed to detect products formed. Cellobiose, sophorose, gentiobiose, laminaribiose, trehalose, glucose, maltose, and isomaltulose were used as substrates at a concentration of 1 mM along with 1 mM glucose-1-phosphase (G1P), in 50 mM MOPS buffer, pH 7. As shown in FIG. 14, the B6 enzyme has activity on sophorose. The TLC samples of FIG. 14 are outlined in Table 10. The dark spot indicated by the arrow shows likely formation of >DP10 glucan formation when sophorose was used as the substrate. The smear above the spot is likely smaller DP glucans that were “too heavy” to be pulled up the slide. This activity by B6 on sophorose was confirmed by anion exchange chromatography, as shown in FIG. 15

TABLE 10

| TLC column | Sample |
|---|---|
| 1 | glucose-1-phosphate (G1P) |
| 2 | sophorose substrate alone |
| 3 | B6 assay results with sophorose substrate |
| 4 | cellobiose substrate alone |
| 5 | B6 assay results with cellobiose substrate |
| 6 | trehalose substrate alone |
| 7 | B6 assay results with trehalose substrate |
| 8 | glucose substrate alone |
| 9 | B6 assay results with glucose substrate |
| 10 | gentiobiose substrate alone |
| 11 | B6 assay results with gentiobiose substrate |
| 12 | maltose substrate alone |
| 13 | B6 assay results with maltose substrate |
| 14 | laminaribiose substrate alone |
| 15 | B6 assay results with laminaribiose substrate |
| 16 | isomaltulose substrate alone |
| 17 | B6 assay results with isomaltulose substrate |
| 18 | MDX - maltodextrin mixture |
| 19 | Blank - B6 enzyme and G1P |

### Example 6—βGP Enzyme Characterization

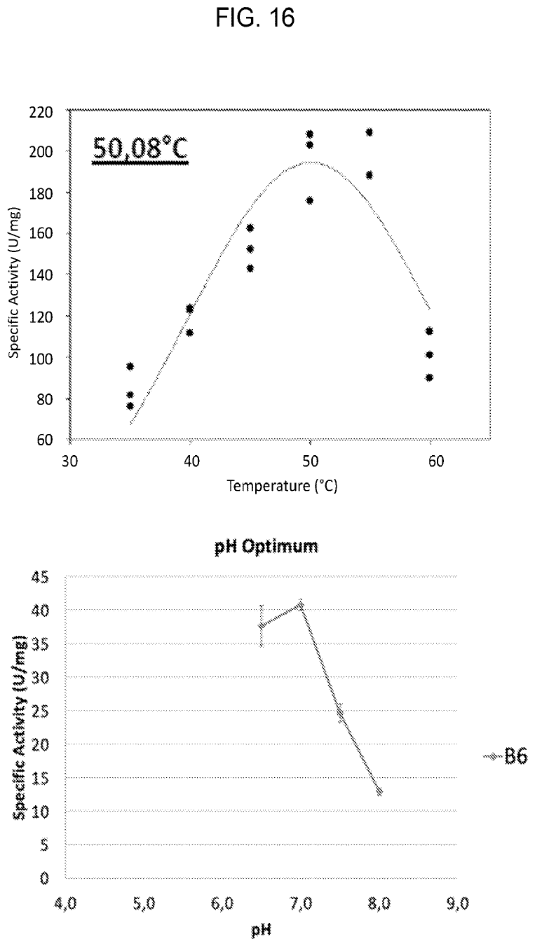

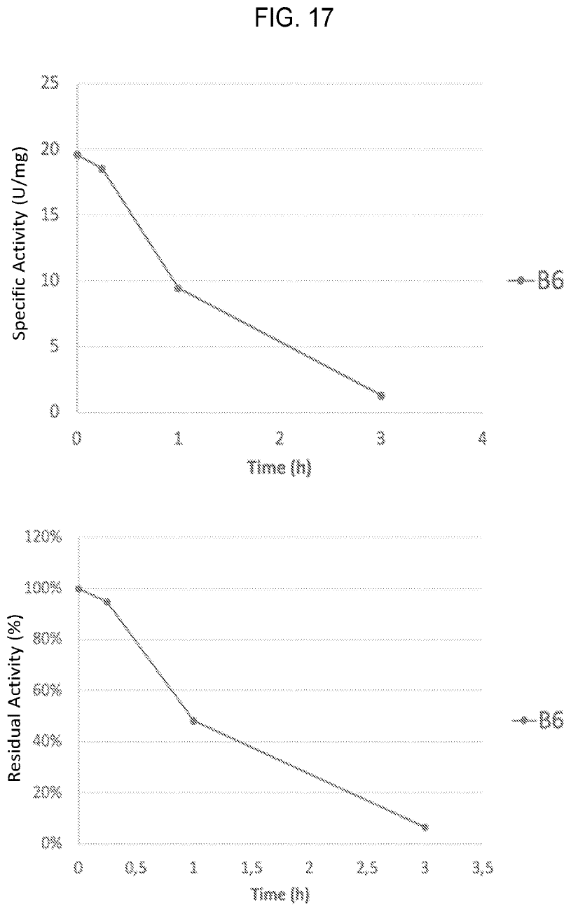

The B6 candidate βGP enzyme was further characterized to identify optimal activity temperature, pH, and kinetic parameters. As demonstrated in FIG. 16, B6 has a peak activity at approximately 50° C. and a pH between 6.5 and 7. FIG. 17 demonstrates the thermostability of the B6 enzyme. After 15 minutes of incubation at 50° C., the B6 enzyme shows a loss of activity, losing more than 80% of its initial activity after 3 hours at 50° C.

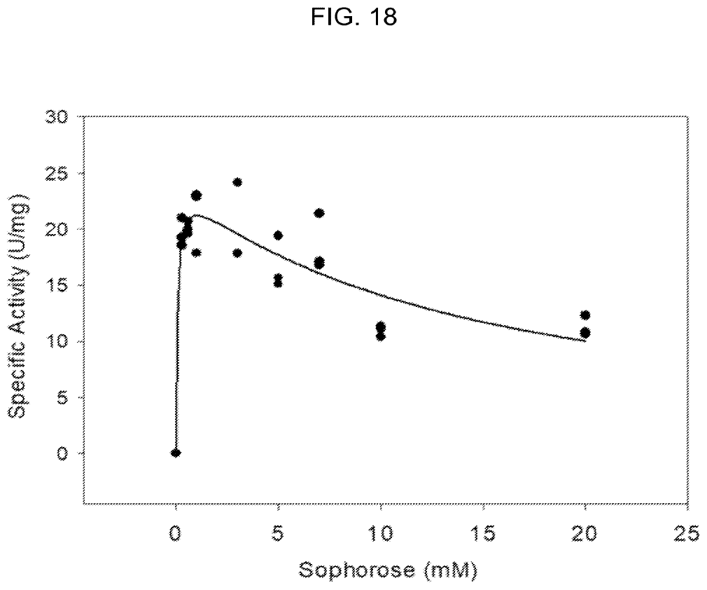

Using the Gawronski phosphate release assay (which measure phosphate concentration), the B6 enzyme showed high activity and substrate inhibition with an inhibitor constant of 14.3 mM sophorose. Michaelis-Menten kinetics are reported in Table 11 and FIG. 18.

TABLE 11

| Enzyme | B6 |
|---|---|
| Kₘ (mM) | 0.07 |
| Vₘₐₓ (U mg⁻¹) | 24 |
| K_{cat} (s⁻¹) | 50 |
| K_{cat}/Kₘ (s⁻¹ mM⁻¹) | 717 |
| Kᵢ (mM) | 14 |

### Example 7—βGP Enzyme Characterization

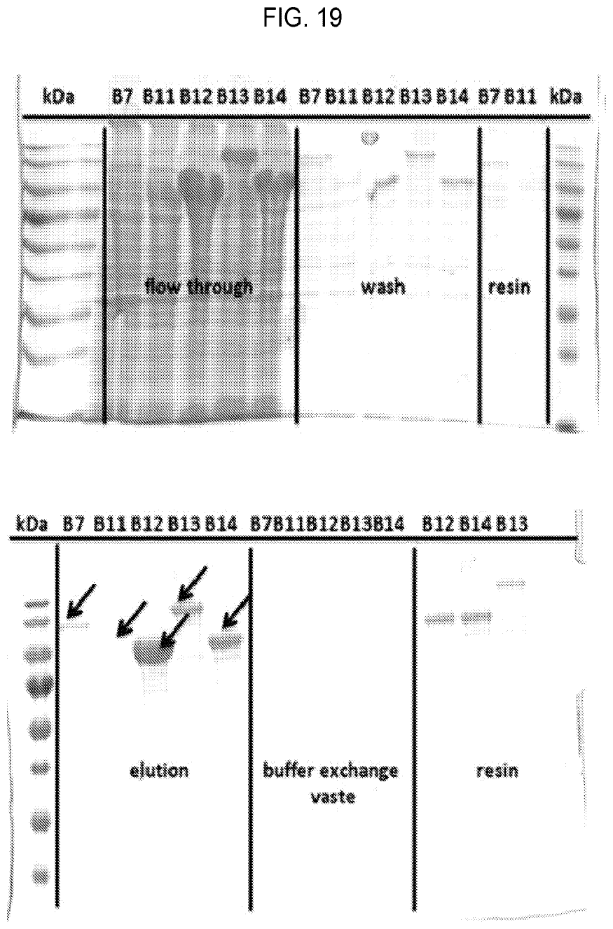

βGP enzyme candidates B7, B11, B12, B13, and B14 were purified using HIS-tag purification (FIG. 19). The B7, B12, B13, and B14 enzymes were present in the eluted samples in reasonable amounts, however purification (and/or expression) of B11 was unsuccessful.

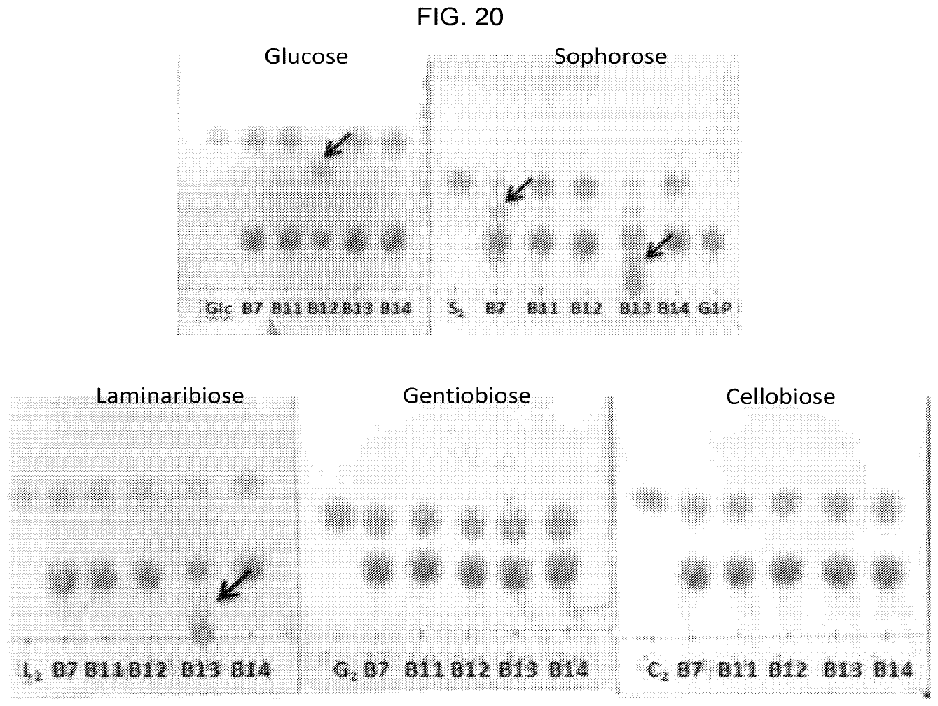

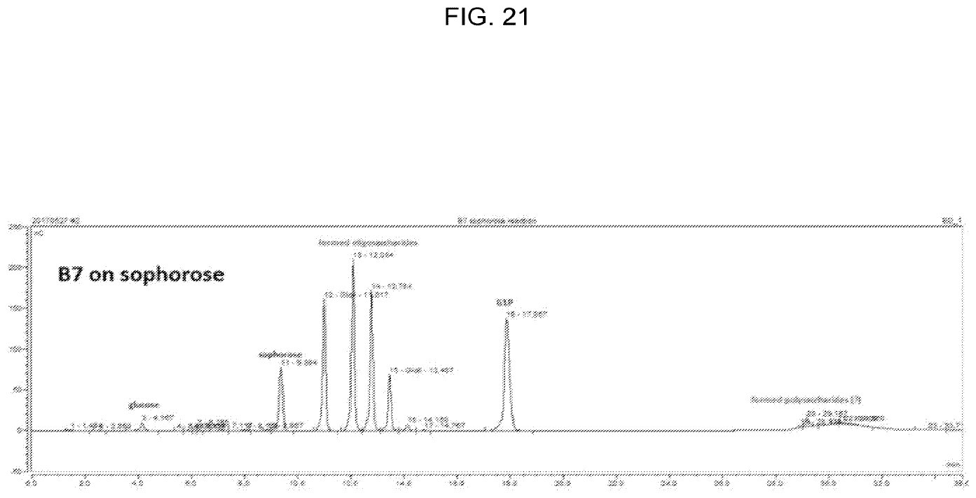

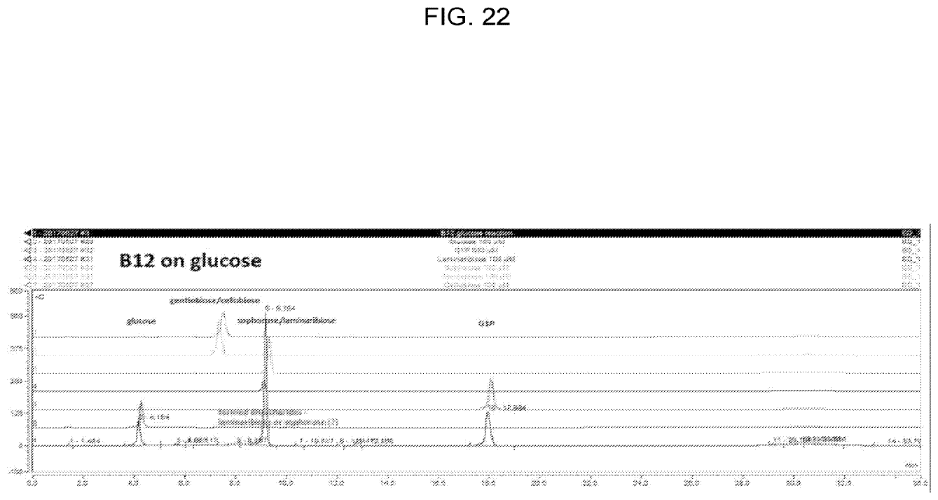

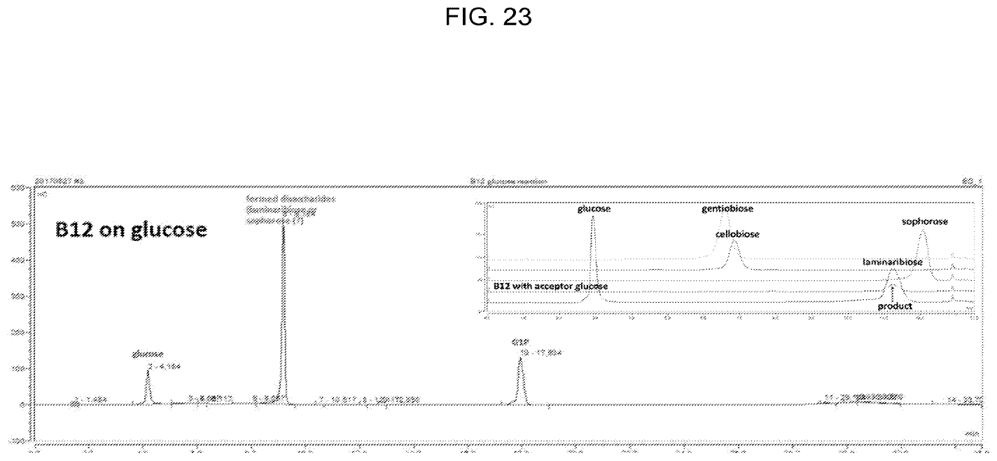

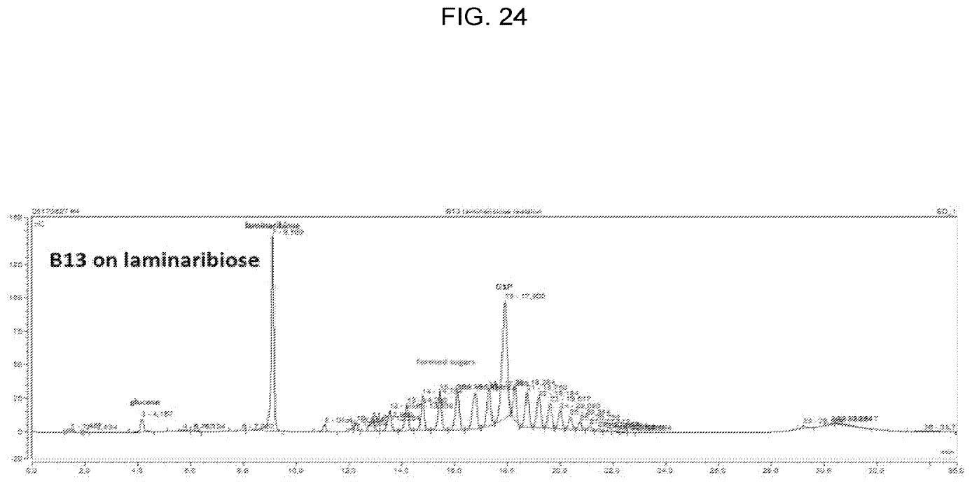

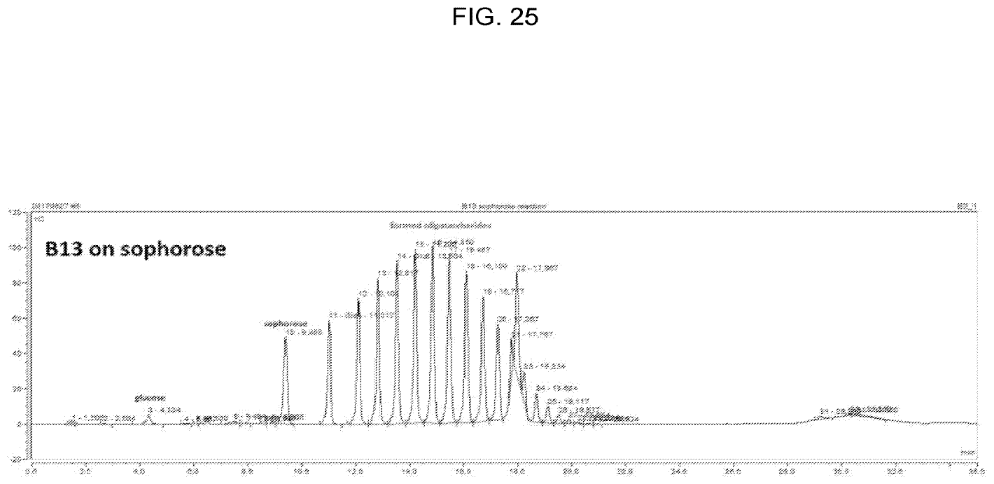

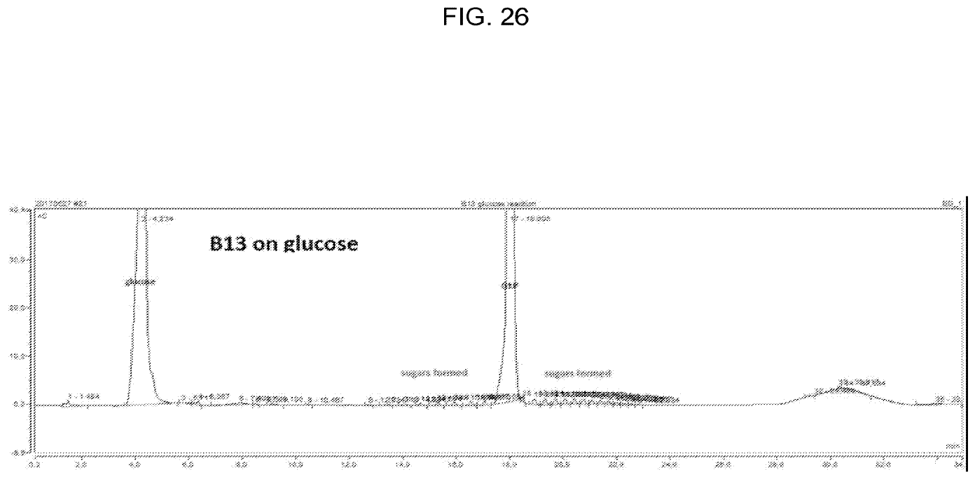

Activity of B7, B11, B12, B13, and B14 was screened on B-disaccharides (cellobiose, gentiobiose, laminaribiose and sophorose), α-disaccharides (isomaltose, maltose, maltulose, sucrose, trehalose) and glucose, focused on product detection by TLC. The reaction was performed at 30° C. for 20 min, after which 1 μl was loaded and developed by TLC. Final concentration of the substrates in the reaction mixture was 5 mM and G1P concentration was 50 mM. The TLC demonstrated that B12 has activity on glucose, by forming disaccharides. B7 and B13 were active on sophorose, B7 by forming mainly oligosaccharides, while B13 formed mainly polysaccharides. B13 also had the activity on laminaribiose by creating primarily polysaccharides, while none of the enzymes (according to TLC analysis) was active on cellobiose and gentiobiose (FIG. 20). Subsequently, the samples were analyzed by anion-exchange chromatography. The results confirmed assumed activities (FIGS. 21-26) and moreover showed that B13 is also active on glucose, forming oligo and polysaccharides. None of the enzymes were active on the a-disaccharide substrates. Anion-exchange chromatography also demonstrated that the disaccharide formed by B12 is laminaribiose.

### Example 8—B6 Characterization on a Mixed Substrate

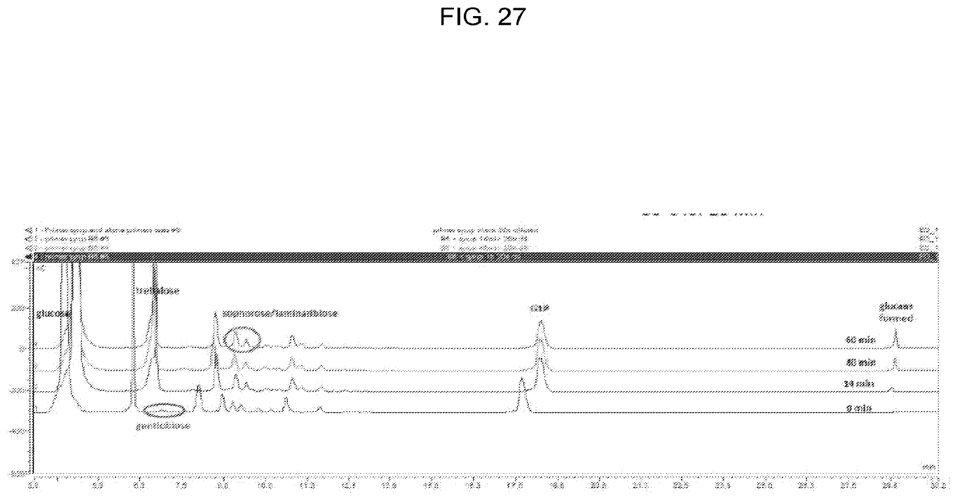

A mixed substrate syrup composition was prepared using the B-glucosidase from Aspergillus niger sold under the tradename “Novozyme 188”. Anion exchange chromatography indicated that the substrate syrup was mostly composed of trehalose, with sophorose, laminaribiose, and gentiobiose present at lower concentrations, as reported in Table 12. The B6 enzyme was active on the substrate syrup, resulting in the production of polysaccharides as shown in FIG. 27.

TABLE 12

| Substrate syrup component | Approximate % |
|---|---|
| Gentiobiose | 2 |
| Laminaribiose | 3.7 |
| Cellobiose | 2 |
| Trehalose | 54 |
| Sophorose | 3.1 |
| Unidentified | 38 |

### Example 9—Synthesis of G1P from Maltodextrins and Phosphate Catalyzed by αGP A8

Various maltodextrins (Cargill, Incorporated and Sigma Aldrich) were used as substrates for the synthesis of G1P. The maltodextrins were of varying average chain lengths and degrees of branching. The A8 αGP enzyme cannot bypass the branches in branched substrates, and therefore, branched substrates are believed to hinder synthesis of G1P. To increase GIP synthesis using the A8 αGP, the maltodextrins were debranched at pH 4.8 and 50° C. prior to running the assay (debranching method described below).

As reported by Weinhäusel et al. (“α-1,4-D-glucan phosphorylase of gram-positive Corynebacterium callunae: isolation, biochemical properties, and molecular shape of the enzyme form solution X-ray scattering,” Biochem J, 1997, 773-783), α-glucan phosphorylases cannot use maltodextrin chains with a degree of polymerization (DP) of 4 or less (DP≤4) as a substrate. As a result, only a fraction of the total glucose units present in maltodextrin are usable by the αGP enzyme.

The initial assays in this example were performed with maltodextrin C DRY MD™ 01910 (“maltodextrin 01910”) from Cargill, Incorporated. Based on calculations using Formula 1, shown below, it is estimated that a maximum of 63% of the maltodextrin 01910 supplied to the A8 catalyzed reaction can be converted to G1P. Formula 1 accounts for the fact that chains of DP=4 (or less) cannot be used as substrates by the A8 enzyme. This calculation is based on the molecular weight distribution obtained using low molecular weight gel permeation chromatography (Table 13).

TABLE 13

| | C*Dry MD01910 |
|---|---|
| Mn | 1375 |
| Mw | 26320 |
| Polydispersity (Mw/Mn) | 19.1 |
| Calculated | 16.2 |

| | | Slicing |
|---|---|---|
| DP range | MW range | (area %) |
| 1−>5 | <909 | 21.5 |
| 6−>9 | 909−>1557 | 23.0 |
| 10−>19 | 1557−>3177 | 10.5 |
| 20−>45 | 3177−>7389 | 8.0 |
| 46−>125 | 7389−>20349 | 12.1 |
| 126−>280 | 20349−>45459 | 10.7 |
| 281−>600 | 45459−>97299 | 8.0 |
| 601−>1500 | 97299−>243828 | 4.2 |
| >1500 | >243828 | 2.0 |

$$Formula 1$$

$$Fraction of usable glucose ( glc ) units in every DP range = \frac{( ( low DP - 4 ) + ( high DP - 4 ) ) / 2}{( low DP + high DP ) / 2}$$

$$Formula 2$$

$$Usable glucose ( \% ) = Slicing area ( 5 ) \times Fraction of usable glc units$$

The fraction of usable glucose units in every DP range calculated using Formula 1 is then used in Formula 2 to determine the total percentage of usable glucose in maltodextrin. The DP ranges and fractions of usable glucose units are reported in Table 14 and usable glucose (glc) units are reported in Table 15.

TABLE 14

| | | | Fraction of usable |
|---|---|---|---|
| DP range | Low DP | High DP | glucose units |
| 1-5 | 1 | 5 | 0.07 |
| 6-9 | 6 | 9 | 0.47 |
| 10-19 | 10 | 19 | 0.72 |
| 20-45 | 20 | 45 | 0.88 |
| 46-125 | 46 | 125 | 0.95 |
| 126-280 | 126 | 280 | 0.98 |
| 281-600 | 281 | 600 | 0.99 |
| 601-1500 | 601 | 1500 | 1.00 |
| >1500 | 1500 | 3000 | 1.00 |

TABLE 15

| DP range | Slicing area (%) | Usable Glc (%) |
|---|---|---|
| 1-5 | 21.5 | 1.4 |
| 6-9 | 23.0 | 10.7 |
| 10-19 | 10.5 | 7.6 |
| 20-45 | 8.0 | 7.0 |
| 46-125 | 12.1 | 11.5 |
| 126-280 | 10.7 | 10.5 |
| 281-600 | 8.0 | 7.9 |
| 601-1500 | 4.2 | 4.2 |
| >1500 | 2.0 | 2.0 |
| Total | | 62.9 |

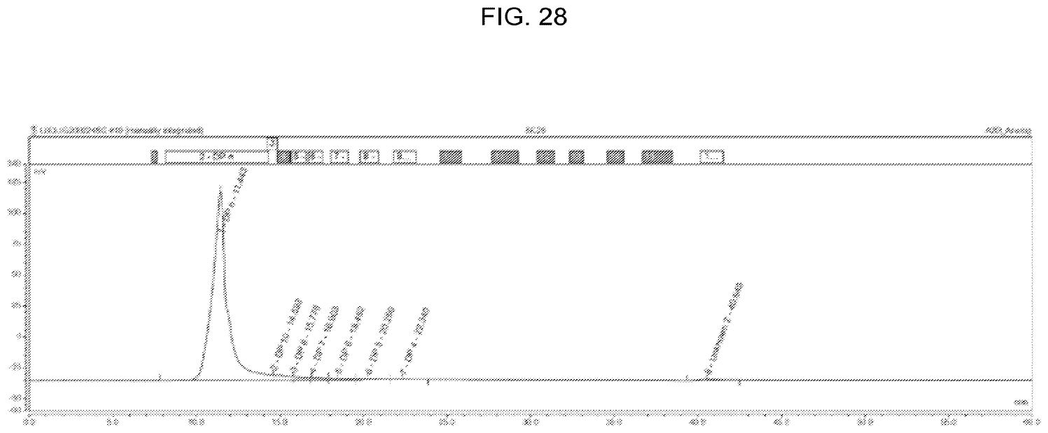

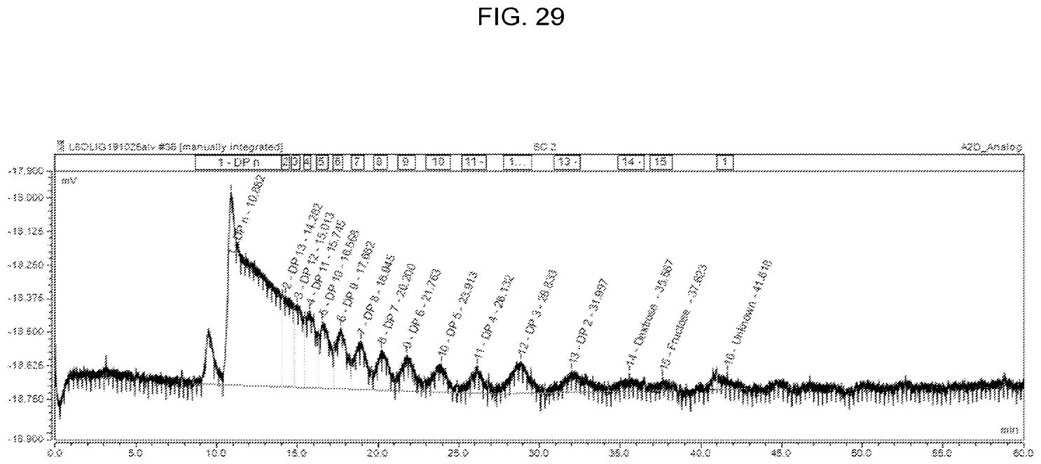

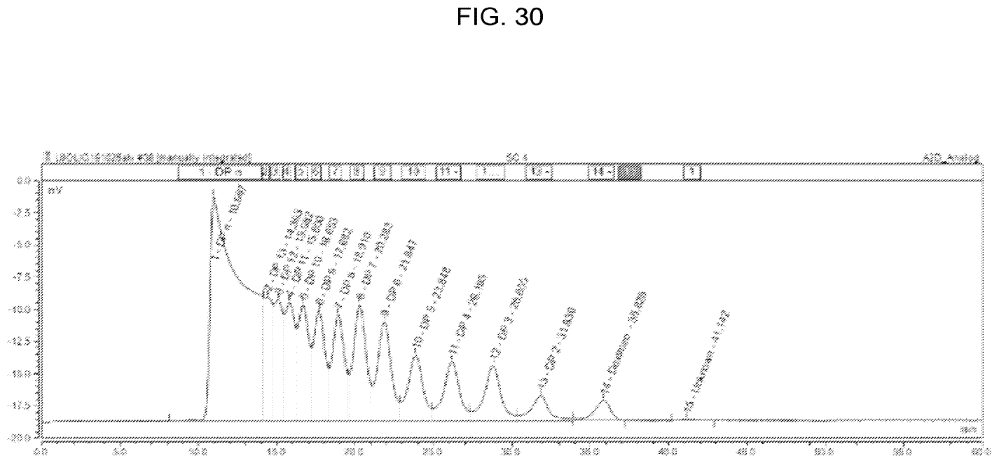

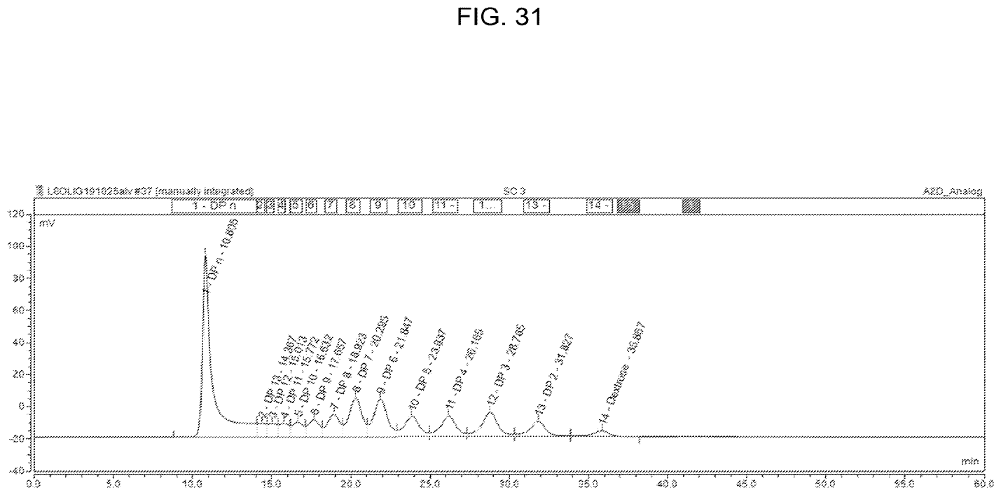

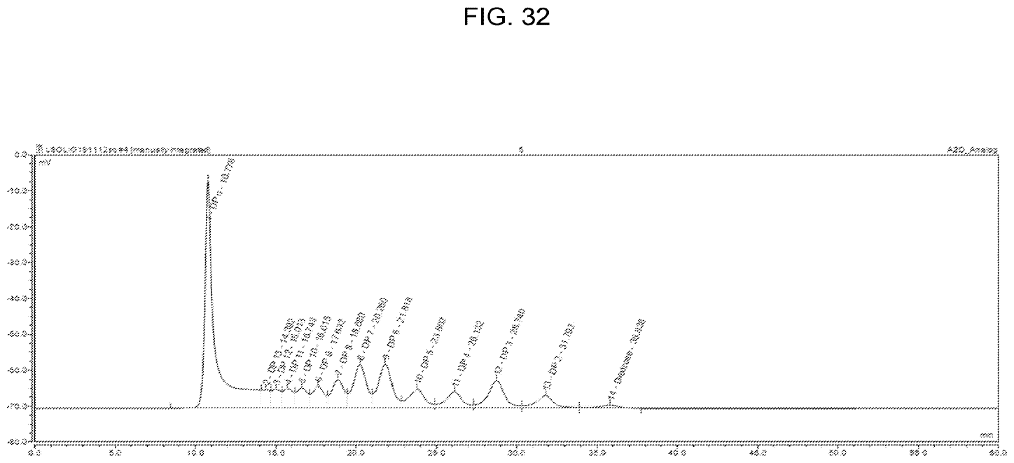

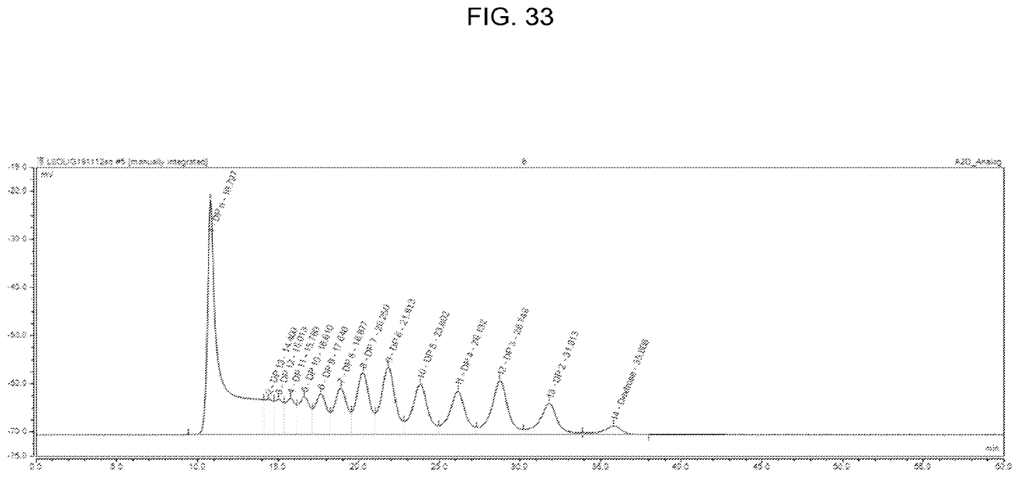

In cases where the maltodextrin substrate is branched, the above calculation may be an overestimation as the calculation does not take into account branching bonds. Six different debranched maltodextrins were investigated as substrates in the enzymatic conversion to produce G1P. After debranching all maltodextrins were analyzed by HPLC fingerprint (Ag+ column) and high molecular weight gel permeation chromatography (GPC). Results are summarized in Tables 16 and 17. Respective chromatograms are shown in FIGS. 28-33.

TABLE 16

| | Area % | | | | | | | | | | | | | | | |
|---|---|---|---|---|---|---|---|---|---|---|---|---|---|---|---|---|
| Samples | DPn | DP13 | DP12 | DP11 | DP10 | DP9 | DP8 | DP7 | DP6 | DP5 | DP4 | DP3 | DP2 | Dextrose | Fructose | Unknown |
| MDX 1910 | 54.79 | — | — | — | 2.07 | 2.88 | 4.8 | 8.16 | 9.14 | 4.09 | 3.7 | 6.55 | 3.09 | 0.54 | — | — |
| Debranched | 37.89 | 2.40 | 2.66 | 3.12 | 3.52 | 4.14 | 5.69 | 9.16 | 9.82 | 4.95 | 4.40 | 7.36 | 3.83 | — | — | 1.07 |
| MDX 1910 | 51.13 | — | — | — | 2.60 | 3.14 | 4.06 | 6.29 | 7.58 | 6.19 | 5.62 | 7.32 | 4.65 | 1.23 | | |
| MDX 1912 | | | | | | | | | | | | | | | | |
| Debranched | 30.20 | 2.84 | 2.89 | 3.31 | 4.06 | 4.60 | 5.74 | 7.79 | 9.00 | 7.45 | 6.69 | 8.57 | 5.30 | — | — | 1.57 |
| MDX1912 | | | | | | | | | | | | | | | | |
| MDX DE 4-7 | 84.36 | — | — | — | 1.59 | 2.03 | 2.15 | 2.60 | 2.11 | 1.41 | 1.41 | 1.33 | 0.57 | 0.29 | — | — |
| Debranched | 35.00 | 4.84 | 5.05 | 5.63 | 5.94 | 6.12 | 6.20 | 7.18 | 6.55 | 4.74 | 4.52 | 4.38 | 2.16 | 1.56 | — | 0.13 |
| MDX DE 4-7 | | | | | | | | | | | | | | | | |
| MDX DE 13-17 | 47.77 | — | — | — | 2.67 | 2.93 | 4.72 | 8.93 | 9.26 | 5.36 | 5.66 | 6.86 | 4.28 | 1.30 | — | — |
| Debranched | 34.99 | 2.23 | 2.17 | 2.50 | 2.99 | 3.75 | 5.29 | 9.57 | 9.82 | 5.93 | 6.21 | 7.61 | 4.96 | 1.98 | — | — |
| MDX 13-17 | | | | | | | | | | | | | | | | |
| Soluble starch | 100.00 | — | — | — | — | — | — | — | — | — | — | — | — | — | — | — |
| Debranched | 46.89 | 6.40 | 4.92 | 5.71 | 5.15 | 4.68 | 4.24 | 3.40 | 3.42 | 2.75 | 2.40 | 4.04 | 3.08 | 1.38 | 0.58 | 0.96 |
| Soluble starch | | | | | | | | | | | | | | | | |
| Zulkowsky | 92.66 | — | — | — | 2.37 | 1.25 | 0.96 | 0.97 | 0.68 | 0.27 | — | — | — | — | — | — |
| starch | | | | | | | | | | | | | | | | |
| Debranched | 74.27 | — | — | — | — | — | 4.10 | 5.15 | 5.12 | 5.77 | 3.24 | — | — | — | — | — |
| Zulkowsky | | | | | | | | | | | | | | | | |
| starch | | | | | | | | | | | | | | | | |

TABLE 17

| | | | | | Debranched | Debranched |
|---|---|---|---|---|---|---|
| GPC high molecular | Debranched | Debranched | Debranched | Debranched | soluble | Zulkowsky |
| weight | MDX 1910 | MDX 1912 | MDX 13-17 | MDX 4-7 | starch | starch |
| Mn | 1209 | 1076 | 4880 | 5698 | 6684 | 3338 |
| Mw | 6113 | 3847 | 21517 | 20273 | 38714 | 8181 |
| Polydispersity | 5.1 | 3.6 | 4.4 | 3.6 | 5.8 | 2.5 |

| | |
|---|---|
| MW range | Slicing (Area %) |

| | | | | | | |
|---|---|---|---|---|---|---|
| <1000 | 29.2 | 35.8 | 1.7 | 0.9 | 0.9 | 4.7 |
| 1000-5000 | 45.2 | 47.7 | 32.7 | 27.8 | 22.9 | 39.6 |
| 5000-25000 | 20.4 | 14.5 | 40.6 | 49.7 | 40.1 | 51.6 |
| 25000-200000 | 5.1 | 2.0 | 24.8 | 21.4 | 34.2 | 4.1 |
| 200000-1000000 | 0.1 | 0.0 | 0.2 | 0.2 | 1.9 | 0.0 |
| 1000000-5000000 | 0.0 | 0.0 | 0.0 | 0.0 | 0.0 | 0.0 |
| >5000000 | 0.0 | 0.0 | 0.0 | 0.0 | 0.0 | 0.0 |

The GPC molecular weight distribution data shows that the number average molecular weight (Mn) of debranched maltodextrin types 01910 and 01912 are approximately 1000 Dalton, while the rest of the maltodextrins had a larger Mn, varying between 5000 and 200,000 Dalton.

Screening of Dry Substrate, Reaction Temperature, Enzyme Dosage, and Molar Ratio Na₂HPO₄/MDX on GIP Production

The production of GIP was carried out in a two-step process: first a debranching step using two types of debranching enzymes to debranch the substrate maltodextrins, followed by the GIP production step. All maltodextrins tested were incubated using a combination of 0.2% pullulanase and 0.1% iso-amylase (based on maltodextrin concentration) during 5 hours at pH 4.8-5 and 50° C.

The GIP synthetic reaction step was intended achieve almost complete consumption of phosphate in the reaction by addition of excess carbohydrate (i.e., maltodextrin). The reaction was performed with various ratios of maltodextrin 01910/Na₂HPO₄ (1:0.25, 1:0.50, and 1:1) at pH 7.

The influence of reaction temperature, enzyme dosage, and type of dry substance in the reaction mixture was evaluated. A sample was taken after 24 hours reaction time. The GIP yield was determined based on the consumed amount of phosphate (i.e., subtracting the measured residual Na₂HPO₄ concentration from the initial phosphate concentration supplied to the reaction mixture). Additionally, GIP yield was determined using the GIP assay described by Silverstein et al. (“Purification and mechanism of action of sucrose phosphorylase,” Journal of Biological Chemistry, 1967, 242 (6): 1338-1346, herein referred to as the “Silverstein GIP assay”) in order to confirm the data based on the consumed amount Na2HPO4. Substrate conversion percentage was determined as a function of GIP yield based on the initial maltodextrin substrate concentration.

Five experiments (Trials 1-5) were performed with a 10 or 20 wt % dry substance (ds) 01910 maltodextrin (MDX) solution. MDX 01910 (6.46 g; 92.81% ds) was dissolved in a total of 20 g demi-water with pH adjustment to pH 4.8 with 0.1M HCl.

0.2% OPTIMAX™ L-1000 pullulanase (0.012 g total based on 6 g dry MDX) and 0.1% iso-amylase (0.006 g total based on 6 g dry MDX) are added to the MDX solution and incubated for 5 hours at 50° C. No deactivation step was carried out between the two successive steps. Next, the debranched MDX solution was subjected to GIP conversion by adding 250, 500 or 1000 mM/L Na₂HPO₄. After homogenization (vortex) of the suspension, pH was measured and if necessary, adjusted to pH 7 using 0.1M NaOH solution. Finally, the reaction was started by adding α-glucan phosphorylase (A8) varying from 64U to 128U enzyme/g dry maltodextrin or 12.8 U/ml to 24U enzyme/ml reaction mixture. Incubation was performed in a thermomixer (950 rpm shaking speed) on a 30 ml (32 g) scale at a temperature of 50 or 60° C. for 24 hours. The enzyme was inactivated after 24 hours incubation by increasing the temperature of the reaction mixture to 90° C. for 5 minutes. The data (reaction conditions; analysis) are summarized in Table 18.

TABLE 18

| | | | | | | | | | | | | Conversion | |
|---|---|---|---|---|---|---|---|---|---|---|---|---|---|
| | | | Reaction | | | Start | Start | | Consumed | Conversion of | | of MDX | |
| | | | temper- | | | amount of | amount | | amount | MDX 01910 as | Determined | 01910 as a | Reaction |
| | % | | ature | Enzyme | | Na₂HPO₄ | of MDX | MR MDX/ | Na₂HPO₄ | a function of | amount G1P | function of | time |
| Trial | ds | pH | ° C. | dosage | | (mM) | (mM) | Na₂HPO₄ | (mM) | Na₂HPO₄(%) | (mM) | G1P (%) | (hrs) |
| 9.1 | 20 | 7 | 60 | 12.8 | U/ml | 230.77 | 1115.3 | 1/0.250 | 83.4 | 7.5 | 72.8 | 6.5 | 24.0 |
| 9.2 | 20 | 7 | 50 | 12.8 | U/ml | 233.59 | 1122.8 | 1/0.250 | 44.1 | 3.9 | 47.4 | 4.2 | 24.0 |
| 9.3 | 10 | 7 | 60 | 12.8 | U/ml | 463.76 | 558.7 | 1/1 | 110.6 | 19.8 | 103.6 | 18.5 | 24.0 |
| 9.4 | 20 | 7 | 60 | 12.8 | U/ml | 450.23 | 1085.0 | 1/0.500 | 112.4 | 10.4 | 127.9 | 11.8 | 24.0 |
| 9.5 | 20 | 7 | 60 | 24 | U/ml | 850.97 | 1038.9 | 1/1 | 247.4 | 23.8 | 127.6 | 12.3 | 24.0 |

The results show higher activity at 60° C. (Trails 9.1 and 9.2). This temperature corresponds to the peak activity temperature of the thermostable α-glucan phosphorylase (A8). An increase in GIP yield/substrate conversion (11.8% for reaction 9.4) was observed when doubling the initial concentration of Na₂HPO₄ (from 250 mM to 500 mM) in reactions performed at 60° C. and 20% ds (Trial 9.1 and 9.4).

Trial 9.3 was carried out with approximately equimolar amounts of Na₂HPO₄ and MDX at a dry substance of 10% and 60° C. and demonstrated a noticeable increase in maltodextrin conversion (18.5%). To further elucidate the contribution of the low dry substrate % versus the equimolar amounts of Na₂HPO₄ and MDX, Trial 9.5 was run with 20% dry substances. To accommodate the increase in both the phosphate concentration and the increase in dry substances %, the reaction used 24 U/mL A8 enzyme. To achieve the 24 U/mL concentration, the A8 enzyme was freeze-dried and added to the reaction in the freeze-dried form. The results of Trial 9.5 shows no further increase in maltodextrin conversion, suggesting that the reaction might be limited by the equilibrium conversion of phosphate (around 20%).

In Trials 9.1-9.4, a good correlation was observed between the analysis of the maltodextrin conversion to GIP and the phosphate consumption analysis. However, Trial 9.5 showed a large difference between the two analysis methods. The REFLECTOQUANT® Phosphate test kit, used to measure residual phosphate, seems to be sensitive to foreign substances in the enzymatic reaction solution. Due to this sensitivity, remaining experiments and examples used the Silverstein GIP assay as a tool for the determination of the generated amount of GIP after reaction.

To shift the equilibrium of the A8 rection in favor of GIP synthesis, addition of an excess of Na₂HPO₄ and a higher reaction temperature (70° C.) were investigated. Samples were taken at regular intervals during the reaction (17-24-48 hours) and assayed for GIP concentration. These assays were performed with a 10 or 15 wt % dry substance 01912 maltodextrin solution. MDX 01912 (1.073 g total; 93.16% ds) was dissolved in a total of 5 g demi-water and pH adjusted to pH 4.8 with 0.1M HCl.

0.2% OPTIMAX™ L-1000 pullulanase (0.002 g total, based on 1 g dry MDX) and 0.1% iso-amylase (0.001 g total, based on 1 g dry MDX) were added to the MDX solution and incubated for 5 hours at 50° C. No deactivation step was carried out prior to the GIP conversion reaction.

Next, the debranched 01912 MDX solution was subjected to GIP conversion by adding 1000 to 2000 mmol/L Pi. After homogenization (vortex) of the suspension, pH is measured and if necessary, adjusted to pH 7 using 0.1M NaOH solution. The reaction was started by addition of the A8 enzyme (64U enzyme/g dry maltodextrin or 12.8 U/ml reaction mixture). The reaction was incubated a thermomixer (950 rpm shaking speed) on a 5 ml (6.6 g) scale at a temperature of 50 or 70° C. for 17, 24, or 48 hours. The enzyme was inactivated by increasing the temperature of the reaction mixture to 90° C. for 5 minutes. The Silverstein GIP assay was used to measure G1P concentration. Data (reaction conditions; analysis) are outlined in Table 19.

The results indicate that an increase in the phosphate concentration up to 2000 mmol/L does not enhance the production rate of GIP. This is likely due to the high concentration of Na₂HPO₄ leading to a viscous insoluble reaction mixture. Likewise, it's likely that an excessively high insoluble salt concentration might cause enzyme deactivation as was the case in Trial 9.7. The most suitable concentration of phosphate in this reaction system was 1000 mmol/L Pᵢ while keeping the maltodextrin concentration constant (Trial 9.6). Evaluation of GIP production as a function of time showed no further increase in G1P synthesis. It seemed that only ≤20% of the phosphate is converted to G1P due to the equilibrium constant of the reaction. Results of Trial 9.8, with a decrease in the dry substance % in combination with an increase in reaction temperature to 70° C., are similar to Trial 9.6.

TABLE 19

| | | | Reaction | | | Start | | Determined | Conversion of MDX | Reaction |
|---|---|---|---|---|---|---|---|---|---|---|
| | % | | temperature | Enzyme | Start amount of | amount of | MR MDX/ | amount of | 01910 as a function | time |
| Trial | ds | pH | ° C. | dosage | Na₂HPO₄ (mM) | MDX (mM) | Na₂HPO₄ | G1P (mM) | of G1P (%) | (hrs) |
| 9.6 | 20 | 7 | 50 | 12.8 U/ml | 918.21 | 913.1 | 1/1 | 158.2 | 17.3 | 17.0 |
| | | | | | 918.21 | 913.13 | | 170.39 | 18.7 | 24.0 |
| | | | | | 918.21 | 913.13 | | 174.18 | 19.1 | 48.0 |
| 9.7 | 20 | 7 | 50 | 12.8/ml | 1680.47 | 835.5 | 1/2 | 35.9 | 4.3 | 17.0 |
| | | | | | 1680.47 | 835.53 | Insoluble | 47.69 | 5.7 | 24.0 |
| | | | | | 1680.47 | 835.53 | | 81.72 | 9.8 | 48.0 |
| 9.8 | 15 | 7 | 70 | 9 U/ml | 779 | 831 | 1/1 | 154.2 | 18.6 | 17.0 |
| | | | | | 779 | 831 | | 155.2 | 18.7 | 24.0 |
| | | | | | 779 | 831 | | 179.7 | 21.6 | 48.0 |

### Screening of Substrate Type, Pi Source, and pH on G1P Production

As reported by Bae et al. (“Facile synthesis of G1P from starch by Thermus Caldophilus GK24 α-glucan phosphorylase,” Process Biochemistry 40 (2005) 3707-3713), soluble starch may be a better substrate, giving a higher yield of G1P than maltodextrins 01910 and 01912. Likewise, a more soluble potassium phosphate substrate, instead of sodium phosphate, may be beneficial.

To investigate G1P synthesis by the A8 enzyme using a soluble starch substrate (5% w/v dry substance) and potassium phosphate, four additional trials were conducted at different soluble starch and KH₂PO₄ molar ratios (1:2.5, 1:3.5) and at two different pH values (pH 7 and 8).

Soluble starch (1.108 g total; 90.25% ds) was dissolved in a total of 10 g demi-water and pH adjusted to pH 4.8 with 0.1M HCl. 0.2% OPTIMAX™ L-1000 pullulanase (0.002 g total, based on 1 g dry sol. starch) and 0.1% iso-amylase (0.001 g total, based on 1 g dry sol. starch) were added to the soluble starch solution and incubated for 5 hours at 50° C. No deactivation step was carried out prior to the G1P synthesis reaction.

Next, the debranched soluble starch (1.725 g debranched soluble starch solution containing 0.1572 g dry weight starch) solution was used in the G1P conversion reaction with 2500 to 3500 mmol/L KH₂PO₄. After homogenization (vortex) of the suspension, pH was measured and adjusted to pH 7 using 0.1M NaOH solution. The reaction was initiated by adding the A8 enzyme (64U enzyme/g dry soluble starch) from the cell crude lysate. The reaction was incubated in a thermomixer (950 rpm shaking speed) on a 10 ml (+/−10.4 g) scale at a temperature of 60° C., within reaction times of 6, 17, and 48 hours. The enzyme was inactivated by increasing the temperature of the reaction mixture up to 90° C. for 5 minutes. G1P concentration in the reaction product was measured using the Silverstein G1P assay. The results and reaction conditions are summarized in Table 20.

The results indicate that G1P yield was higher in reaction runs at pH 7. Trial 9.10 showed high soluble starch conversion (24% after 17 hours) with a soluble starch: KH₂PO₄ molar ratio of 1:3.5. This is a 5% G1P yield increase in comparison to previous reaction trials with Na₂HPO₄ and maltodextrin 01912. The cause of the decrease in G1P yield after 24 hours, as observed in Trials 9.9 and 9.10, is not completely clear. The A8 enzyme cell lysis debris may contain a contaminant (e.g., a phosphatase) which is responsible for the break-down of G1P or it may be due to the presence of a precipitate in the non-homogeneous crude enzyme sample.

TABLE 20

| | | | Reaction | | Start amount | Start amount | MR soluble | Determined | Conversion of | Reaction |
|---|---|---|---|---|---|---|---|---|---|---|
| | % | | temperature | Enzyme | of KH₂PO₄ | of soluble | starch/ | amount of | soluble starch as a | time |
| Trial | ds | pH | ° C. | dosage | (mM) | starch (mM) | KH₂PO₄ | G1P (mM) | function of G1P (%) | (hrs) |
| 9.9 | 5 | 7 | 60 | 1.3 U/ml | 222 | 87.6 | 1/2.5 | 16.4 | 18.7 | 17.0 |
| | | | | | 222 | 87.6 | | 9.57 | 10.9 | 24.0 |
| 9.10 | 5 | 7 | 60 | 1.04 U/ml | 260 | 71.3 | 1/3.5 | 17.15 | 24.1 | 17.0 |
| | | | | | 260 | 71.3 | | 10.2 | 14.3 | 24.0 |
| 9.11 | 5 | 8 | 60 | 2.6 U/ml | 222 | 84.7 | 1/2.5 | 7.15 | 8.4 | 6.0 |
| | | | | | 222 | 84.7 | | 11.03 | 13 | 24.0 |
| 9.12 | 5 | 8 | 60 | 2.08 U/ml | 253 | 71.06 | 1/3.5 | 4.87 | 6.9 | 6.0 |
| | | | | | 253 | 71.06 | | 6.5 | 9.1 | 24.0 |

TABLE 21

| | | | | | | | MR | | Conversion of | | Hours |
|---|---|---|---|---|---|---|---|---|---|---|---|
| | | | Reaction | | Start amount | Start amount | soluble | Determined | soluble starch | Reaction | according |
| | % | | temperature | Enzyme | of KH₂PO₄ | of soluble | starch/ | amount of | as a function | time | to U/ml |
| Trial | ds | pH | ° C. | dosage | (mM) | starch (mM) | KH₂PO₄ | G1P (mM) | of G1P (%) | (hrs) | addition |
| 9.13 | 5 | 7 | 60 | 3.3 U/ml | 253 | 72.0 | 1/3.5 | 29.63 | 41.2 | 5.0 | |
| | | | | | 253 | 72.0 | | 32.2 | 44.8 | 8.0 | |
| 9.14 | 5 | 7 | 60 | 9.9 U/ml | 260 | 65.3 | 1/3.5 | 20.56 | 31.5 | 1.0 | 1*3 = 3 |
| | | | | | 260 | 65.3 | | 18.95 | 29.0 | 2.0 | 6 |
| | | | | | 260 | 65.3 | | 18.74 | 28.7 | 3.0 | 9 |
| | | | | | 260 | 65.3 | | 20.61 | 31.6 | 4.0 | 12 |
| | | | | | 260 | 65.3 | | 20.15 | 30.9 | 5.0 | 15 |
| | | | | | 260 | 65.3 | | 18.54 | 28.4 | 6.0 | 18 |
| | | | | | 260 | 65.3 | | 19.1 | 29.2 | 7.0 | 21 |
| | | | | | 260 | 65.3 | | 17.65 | 27.0 | 8.0 | 24 |

Based on the results of Trail 9.10, an additional reaction was performed (Trial 9.13) that was identical to Trail 9.10 but had shorter reaction times (5 hrs and 8 hrs) and tripled amounts (192 U/g dry sol starch) of the A8 αGP enzyme. Trial 9.13 showed very high conversion degrees, up to 44.8% after 8 hours. In trial 9.14, A8 enzyme concentration was tripled. The reaction conditions of Trail 9.14 were equivalent to Trail 9.13, but the incubation time was varied based on A8 concentration.

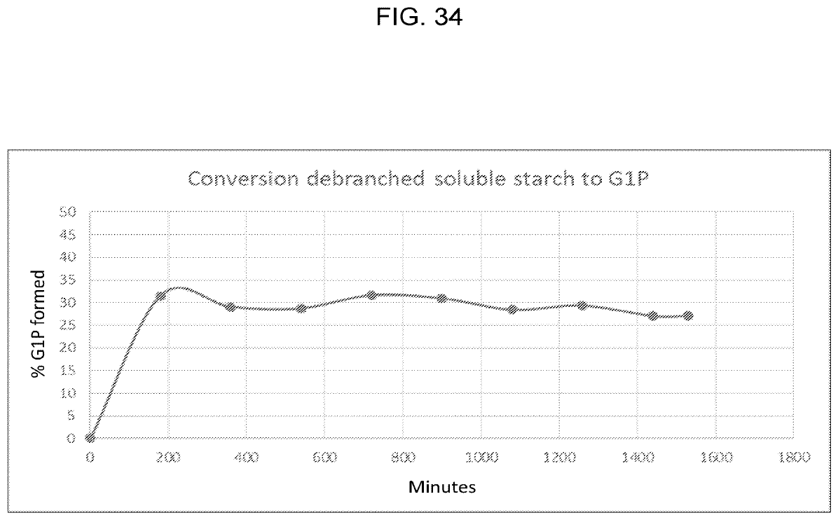

Increasing reaction time demonstrated an equilibrium in the formation of G1P over the period from 3 to 24 hours. An approximately +30% conversion equilibrium of soluble starch into G1P was reached over a time period of 24 hours (see Table 21 and FIG. 34).

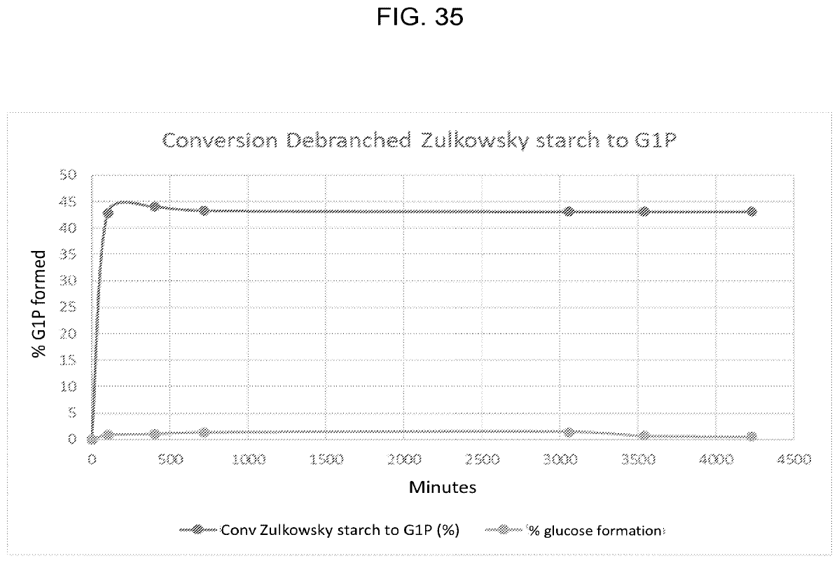

In addition to soluble starch, other substrates with longer average chain lengths were tested. Commercially available Zulkowsky starch (i.e., potato starch treated with glycerol at 190° C., see K. Zulkowsky, “Verhalten der Starke gegen Glycerin,” Ber. Deutsch. Chem. Ges., 13, 1395, 1880)), as well as two other maltodextrin types with varying dextrose equivalents (dextrose equivalents of 13.0-17.0 and 4.0-7.0, from Sigma-Aldrich), were evaluated using reaction conditions similar to Trial 9.14. All substrates were debranched before the A8 catalyzed reaction. The Zulkowsky starch reaction (Trial 9.15) was conducted using the A8 αGP (192U enzyme/g dry Zulkowsky starch) cell debris lysate. Trials using the 13-17 and 4-7 dextrose equivalent maltodextrins (Trials 9.16 and 9.17, respectively) were conducted using a cell debris suspension with A8 inclusion bodies. G1P concentration in the reaction product was evaluated using the Silverstein G1P assay and the presence of glucose was measured using the GOPOD-FORMAT procedure from Megazyme. Formation of glucose in these assays was likely due to the presence of contaminants in the enzyme preparations. The results and reaction conditions are summarized in Tables 22, 23, 24, 25, and 26. FIGS. 35-37 shows G1P and glucose production during the course of the reaction of Trails 9.15, 9.16 and 9.17, respectively.

Under the reaction conditions of Trial 9.15, a G1P yield of 44% is reached and maintained over an incubation period of 7 to 10 hours, also confirming thermostability of the active A8 enzyme. The use of Zulkowsky starch increases G1P yield by a factor of 1.5 compared to Trials 9.16 and 9.17. Given that the amount of glucose formed stays at a minimal level, it's likely that the crude A8 cell lysate used in the assays of Trail 9.15 was free of phosphatase contamination.

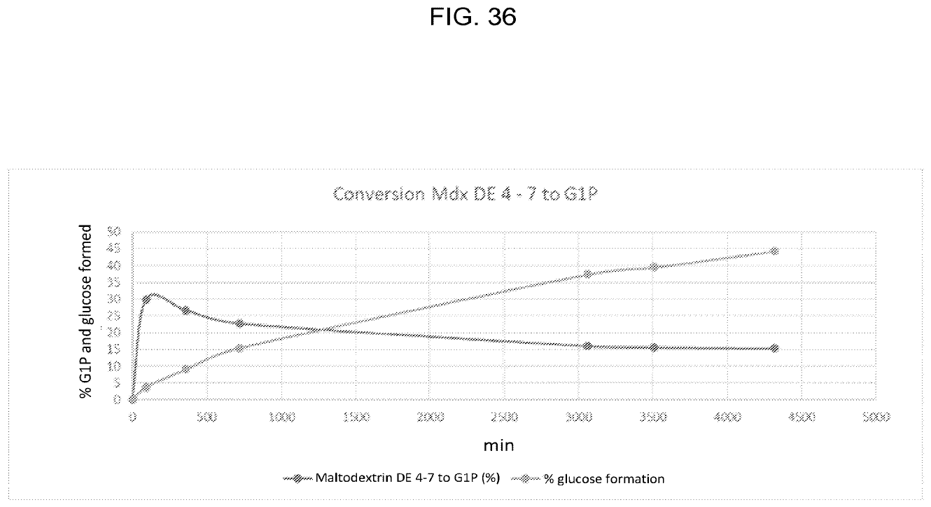

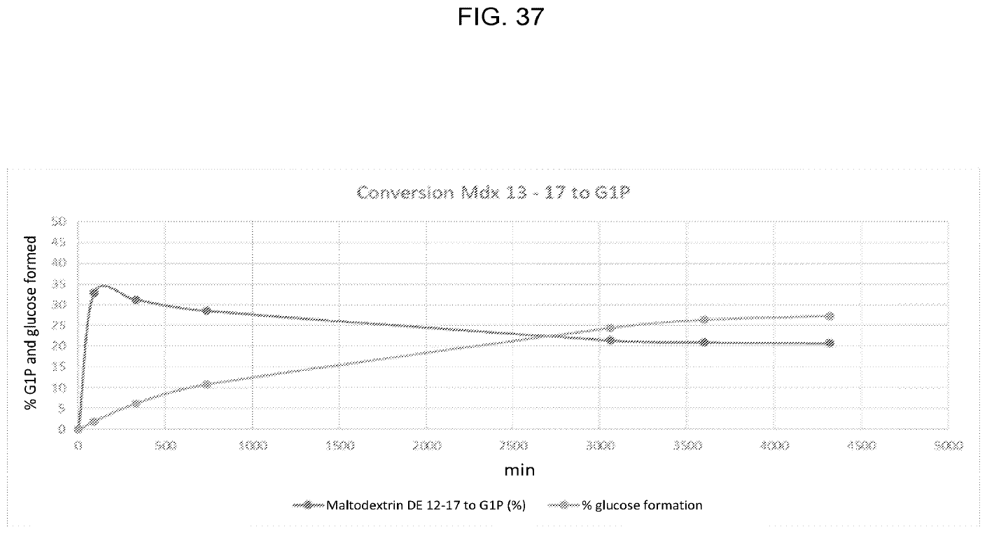

However, it's likely that the cell debris suspension with A8 inclusion bodies used in Trails 9.16 and 9.17 was contaminated with a phosphatase (converting G1P to glucose), as glucose levels rose throughout the assay timepoints (see Tables 24, 25, and 26 and FIGS. 36 and 37). This is likely what contributed to the overall lower yield of G1P.

TABLE 22

| | | | Reaction | | Start | | MR | | Conversion of | | | Hours |
|---|---|---|---|---|---|---|---|---|---|---|---|---|
| | | | temper- | | amount | Start amount | Zulkowsky | Determined | Zulkowsky starch | | | according |
| | % | | ature | Enzyme | of KH₂PO₄ | of Zulkowsky | starch: | amount of | as a function of | | | to U/ml |
| Trial | ds | pH | ° C. | dosage | (mM) | starch (mM) | KH₂PO₄ | G1P (mM) | G1P (%) | Reaction time | | addition |
| 9.15 | 5 | 7 | 60 | 9.9 U/ml | 235 | 68.3 | 1/3.5 | 29.22 | 42.8 | 34 | min | 1 h 42 |

| 235 | 68.3 | 30.02 | 44 | 2 hr 15 min | | 6 hrs 45 | |
|---|---|---|---|---|---|---|---|
| 235 | 68.3 | 29.59 | 43.3 | 4 | hr | 12 | h |
| 235 | 70.15 | 30.23 | 43.1 | 17 | hr | 51 | h |

| | | | | | | |
|---|---|---|---|---|---|---|
| 235 | 70.15 | 30.22 | 43.1 | 19 hr 40 min | 59 | h |
| 235 | 70.15 | 30.25 | 43.1 | 23 hr 30 min | 70.5 | h |

TABLE 23

| Reaction | Conversion of Zulkowsky | % Glucose |
|---|---|---|
| time (min) | starch to GIP (%) | formation |
| 0 | 0 | 0 |
| 102 | 42.8 | 0.84 |
| 405 | 44 | 1.04 |
| 720 | 43.3 | 1.34 |
| 3060 | 43.1 | 1.43 |
| 3540 | 43.1 | 0.66 |

TABLE 24

| | | | Reaction | | Start | Start amount | MR | Determined | Conversion of | | | Hours |
|---|---|---|---|---|---|---|---|---|---|---|---|---|
| | | | temper- | | amount | of Malto- | Malto- | amount | Maltodextrin | | | according |
| | % | | ature | Enzyme | of KH₂PO₄ | dextrin | dextrin: | G1P | as a function | Reaction | | to U/ml |
| Trial | ds | pH | ° C. | dosage | (mM) | (mM) | KH₂PO₄ | (mM) | of G1P (%) | time | | addition |
| 9.16 | 5 | 7 | 60 | 9.9 U/ml | 270 | 77 | 1:3.5 | 22.91 | 29.8 | 30 | min | 1 h 30 min |
| MDX DE | | | | | 270 | 77 | | 20.52 | 26.6 | 2 | hrs | 6 hrs |
| 4-7 | | | | | 270 | 77 | | 17.47 | 22.7 | 4 | hrs | 12 hrs |
| | | | | | 270 | 69.77 | | 11.18 | 16.0 | 17 | hrs | 51 hrs |
| | | | | | 270 | 69.77 | | 10.79 | 15.5 | 19.5 | hrs | 58 hrs 30 min |
| | | | | | 270 | 69.77 | | 10.66 | 15.3 | 24 | hrs | 72 hrs |
| 9.17 | 5 | 7 | 60 | 9.9 U/ml | 275 | 79.8 | 1:3.5 | 26.24 | 32.9 | 30 | min | 1 h 30 min |
| MDX DE | | | | | 275 | 79.8 | | 24.9 | 31.2 | 5.5 | hrs | 5 hrs 30 h |

| | | | | | | |
|---|---|---|---|---|---|---|
| 13-17 | 275 | 79.8 | 22.76 | 28.5 | 4 hrs 6 min | 12 hrs 18 |

| | | | | | | |
|---|---|---|---|---|---|---|
| 266 | 77.1 | 16.52 | 21.4 | 17 | hrs | 51 hrs |
| 266 | 77.1 | 16.11 | 20.9 | 20 | hrs | 60 hrs |
| 266 | 77.1 | 15.99 | 20.7 | 24 | hrs | 72 hrs |

TABLE 25

| Reaction | Maltodextrin DE 4-7 | % glucose |
|---|---|---|
| time (min) | to G1P (%) | formation |
| 0 | 0 | 0 |
| 90 | 29.8 | 3.78 |
| 360 | 26.7 | 9.06 |
| 720 | 22.7 | 15.48 |
| 3060 | 16 | 37.39 |
| 3510 | 15.5 | 39.48 |
| 4320 | 15.3 | 44.32 |

TABLE 26

| Reaction | Maltodextrin DE 13-17 | % glucose |
|---|---|---|
| time (min) | to G1P (%) | formation |
| 0 | 0 | 0 |
| 90 | 32.9 | 1.94 |
| 330 | 31.2 | 6.23 |
| 738 | 28.5 | 10.84 |
| 3060 | 21.4 | 24.4 |
| 3600 | 20.9 | 26.36 |
| 4320 | 20.7 | 27.3 |

### Example 10—Synthesis of β-Glucan from G1P and a Primer Molecule Catalyzed by β-Glucan Phosphorylase B6

This example demonstrates the production of a 1,2-B-oligoglucan using the B6 βGP enzyme. For this example, the B6 enzyme is supplied to the reaction as a crude cell extract to stabilize the enzyme at the elevated reaction temperatures. This crude cell extract also includes some residual glucose which can act as a primer molecule during β-glucan synthesis. The oligoglucan assembly needs a starting or “primer” molecule upon which the oligoglucan will be built out be transfer of the glucose molecule. Crude cell extracts will include residual glucose to act as this primer molecular, but if the B6 enzyme is purified from the cell extract (e.g., His-tag purification and the like), a separate primer molecule (sophorose or glucose) is added to the reaction.

### Effect of Substrate Concentration on β-Glucan Synthesis

In first instance, the effect of the substrate (G1P) concentration in this second reaction of the process, is evaluated. Three 30 ml reactions were performed starting from 0.5M, 1M, or 1.5M G1P (Sigma-Aldrich) to evaluate the effect of substrate concentration on β-glucan synthesis. These G1P concentrations correspond to 14%, 26%, and 40% dry substances, respectively.

Alpha-D-glucose-1-phosphate (G1P, 98% purity) was dissolved in ˜30 ml phosphate buffer 100 mM pH 7, pH adjusted with HCl 1M. 0.1 mL of Dithiotreitol (DTT) 1M was added to protect the enzyme against oxidation. The reaction mixture was prepared in a 50 ml FALCON test tube and mixed through a Vortex in order to obtain a homogeneous solution.

Activity and protein content of the crude B6 enzyme cell lysate prepared by sonication was measured. A corresponding activity of 30 U/ml enzyme solution is determined with the Gawronski phosphate release test. A protein content of 10 mg protein/ml is quantified with the Pierce BCA kit from Thermofisher.

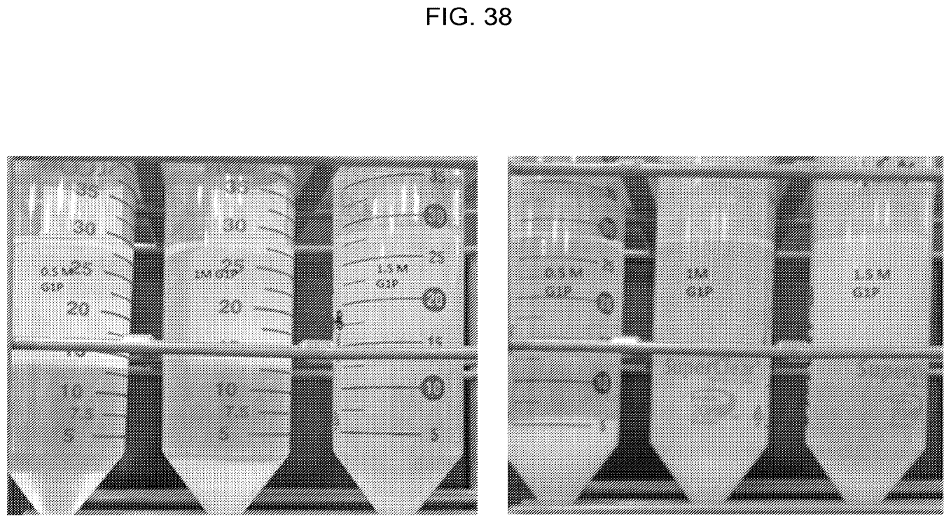

3 ml (90U) β-glucan phosphorylase B6 was added to the 30 ml reaction mixture, resulting in an enzyme concentration of 3U enzyme per ml substrate in the reaction solution. No primer syrup or sophorose (primer molecule) was added due to the presence of residual glucose in the crude cell lysate. The tubes were placed in a pre-heated thermomixer (900 rpm shaking speed) at 40° C. Samples were taken after an incubation period of 24 hours and heated at 90° C. for 10 minutes to inactive the B6 enzyme. A precipitate (0.6 wt % based on starting weight of G1P) was noticed during reaction and after deactivation of the enzyme. This haze, which is not soluble in H₂O/DMSO (10%/90%) solution, is probably coming from the enzyme and not associated with formation of the β-glucan. (See FIG. 38).

To evaluate the difference between sonication and homogenization applied during cell lysis, a second set of reactions (3) were executed under identical reaction conditions but supplying homogenized lysed β-glucan phosphorylase to the reaction mixture. An enzyme activity of 22.32 U/ml and a 8.2 mg protein/ml enzyme is measured.

Resulting β-glucan concentrations for each reaction were determined based on the concentration of residual G1P (phosphoglucomutase/glucose-6-dehydrogenase assay) and the concentration of glucose (glucose oxidase/peroxidase assay) in the reaction product. The molecular weight distribution of the resulting β-glucan was determined using high molecular weight GPC.

Because the phosphate, G1P, and β-glucan peaks overlap on the high molecular weight GPC (Table 27), G1P and phosphate need to be removed from the reaction product prior to analysis. The reaction product was treated with 0.5% acid phosphatase from wheat germ (Aldrich) for 24 hours at 37° C. and pH 4.8. Following acid phosphatase treatment, phosphate and glucose were removed by dialysis. G1P and phosphate may also be removed by use of a guard column prior to the GPC analytical column.

TABLE 27

| | G1P | Phosphate | Treated reaction |
|---|---|---|---|
| | standard | standard | mixture |
| Mn | 1427 | 1163 | 4221 |
| Mw | 1553 | 1334 | 9757 |
| Polydispersity | 1.1 | 1.1 | 2.3 |

| MW range | Slicing (Area %) | | |
|---|---|---|---|
| <1000 | 5.5 | 22.7 | 1.8 |
| 1000-5000 | 945 | 77.3 | 37.0 |
| 5000-25000 | — | — | 55.1 |
| 25000-200000 | — | — | 62 |
| 200000-1000000 | — | — | — |
| 1000000-5000000 | — | — | — |
| >5000000 | — | — | — |

β-glucan yield, glucose concentration, and oligosaccharide molecular weight (MW) distribution of the reactions described above are summarized in Tables 28 and 29.

TABLE 28

| | | | HPLC fingerprint AG⁺ | | Initial | Residual | | Used | Conv | G1P conv | G1P conv |
|---|---|---|---|---|---|---|---|---|---|---|---|
| | Temp | | Dpn | Dextrose + | G1P | G1P | Glucose | G1P | G1P | to glucose | to β-glucan |
| Trial | (° C.) | Method of lysis | (%) | G1P (%) | (mM) | (mM) | (mM) | (mM) | (%) | (%) | (%) |
| 10.1 | 40 | sonication | 73.6 | 25.5 | 455 | 152.5 | 46.6 | 302.5 | 66.5 | 9.28 | 57.2 |
| 10.2 | 40 | sonication | 83.8 | 15.5 | 858 | 283.3 | 48.9 | 574.7 | 57.47 | 4.89 | 52.58 |
| 10.3 | 40 | sonication | 43 | 53.3 | 1500 | 1380 | 29.66 | 119.4 | 7.96 | 1.98 | 5.98 |
| 10.4 | 40 | homogenization | 73.5 | 23.2 | 500 | 286.6 | 27.68 | 213.4 | 42.7 | 5.54 | 37.14 |
| 10.5 | 40 | homogenization | 82.05 | 17.5 | 1000 | 631.6 | 34.6 | 368.4 | 36.8 | 3.46 | 33.4 |
| 10.6 | 40 | homogenization | 30.7 | 66.4 | 1500 | 1390 | NP | 110 | 7.3 | — | — |

TABLE 29

| | | GPC HMW system | | MW range | | | | | | |
|---|---|---|---|---|---|---|---|---|---|---|
| | | DP | MW | <1000 | 1000-5000 | 5000-25000 | 25000-200000 | 200000-1000000 | 1000000-5000000 | >5000000 |
| Trial | Method of lysis | range | (kDa) | area % | area % | area % | area % | area % | area % | area % |
| 10.1 | sonication | 59.13 | 9757 | 1.8 | 37 | 55.1 | 6.2 | — | — | — |
| 10.2 | sonication | 221.29 | 36513 | 0 | 4.5 | 40.4 | 54.8 | 0.4 | — | — |
| 10.3 | sonication | 75.96 | 12533 | 3.4 | 46.9 | 32.4 | 17.3 | — | — | — |
| 10.4 | homogenization | 189.14 | 31208 | 1.0 | 8.0 | 40.4 | 48.4 | 0.3 | — | — |
| 10.5 | homogenization | 630.57 | 104044 | 0.6 | 7.8 | 4.1 | 76.9 | 10.7 | — | — |

| | | |
|---|---|---|
| 10.6 | homogenization | No data available |

A higher β-glucan yield (57.2%) was obtained in the reaction with a lower initial substrate concentration (0.5M G1P, Trail 10.1). However, the glucose concentration in the reaction product was doubled (9.28%) Compared to Trial 10.2. The Trail 10.3 reaction with an initial G1P concentration of 1.5M showed low β-glucan conversion (5.98%). This may be due to the high dry substances concentration in the reaction mixture that is too viscous for efficient enzyme activity.

When comparing the reactions run with enzymes prepared under different lysis methods, sonication resulted in a higher β-glucan conversion percentage (52.6% in Trial 10.2 compared to 33.4% in Trial 10.5). However, reactions with the homogenization prepared crude extract resulted in the production of larger β-glucans, with a degree of polymerization (DP) up to 630 (Trial 10.5). Overall, the data demonstrates that crude enzyme extract prepared from either sonication or homogenization produce the β-glucan products.

### Effect of Temperature and Phosphatase Inhibitors on β-Glucan Synthesis

To assess the effects of a phosphatase inhibitors and the effects of the absence of glucose as a primer, the B6 enzyme was pre-incubated with SUMIZYME® GOP glucose oxidase and catalase cocktail and/or sodium molybdate. 200 mM sodium molybdate and/or 10 mg (per ml B6 enzyme) SUMIZYME® GOP glucose oxidase and catalase cocktail was incubated with the B6 enzyme for 5 hours at room temperature (approximately 22° C.) at a pH between 6.5 and 7.

Following B6 enzyme preincubation with the phosphatase inhibitor, reactions were prepared including 3 U/ml B6 enzyme, 1M G1P, 100 mM phosphate buffer pH 7, and 0.1 mL DTT per 30 mL reaction mixture. The reactions also included the sodium molybdate and/or SUMIZYME® GOP glucose oxidase and catalase cocktail included with the B6 enzyme preincubation (i.e., inhibitors were not removed prior to starting the reactions). Reactions were run at either 40° C. or 50° C. for 24 hours. β-glucan yield, glucose concentration, and MW distribution of the resulting β-glucans are reported in Tables 30 and 31.

TABLE 30

| | | | | | | | | | | G1P | G1P |
|---|---|---|---|---|---|---|---|---|---|---|---|
| | | | Inhibitor type | | Initial | Residual | | Used | Conv | conv to | conv to |
| | Temp | Method of | SUMIZYME ® | Sodium | G1P | G1P | Glucose | G1P | G1P | glucose | β-glucan |
| Trial | (° C.) | lysis | GOP | molybdate | (mM) | (mM) | (mM) | (mM) | (%) | (%) | (%) |
| 10.7 | 40 | sonication | + | | 1000 | 423.82 | 0.47 | 576.2 | 57.62 | 0.047 | 57.57 |
| 10.8* | 40 | sonication | + | | 1000 | 378.92 | 0.5 | 621 | 62.1 | 0 | 61.6 |
| 10.9 | 50 | sonication | + | | 1000 | 461.37 | 16.56 | 538.6 | 53.86 | 1.66 | 52.2 |
| 10.10 | 40 | sonication | + | + | 1000 | 520.5 | 0.23 | 479.5 | 47.95 | 0.02 | 47.92 |
| 10.11 | 40 | sonication | | + | 1000 | 392.52 | 11.34 | 607.5 | 60.75 | 1.1 | 59.62 |
| 10.12 | 40 | sonication | + | | 1476 | 1401.4 | 26.69 | 74.69 | 4.98 | 1.7 | 3.3 |

*3 U/mL B6 enzyme supplementation at 17 h

TABLE 31

| | | GPC HMW system | | MW range (slicing area %) | | | | | | |
|---|---|---|---|---|---|---|---|---|---|---|
| | Method of | DP | MW | | 1000- | 5000- | 25000- | 200000- | 1000000- | |
| Trial | lysis | range | (kDa) | <1000 | 5000 | 25000 | 200000 | 1000000 | 5000000 | >5000000 |
| 10.7 | sonication | 64.44 | 10633 | 0.6 | 7.4 | 5 | 74.4 | 12.5 | — | — |
| 10.8 | sonication | No data available | | | | | | | | |
| 10.9 | sonication | 750.08 | 123764 | 0.2 | 6.5 | 2.9 | 72.7 | 17.6 | — | — |
| 10.10 | sonication | 554.77 | 91537 | 0.6 | 15.1 | 5 | 69.8 | 9.5 | — | — |
| 10.11 | sonication | 566.07 | 93401 | 0.2 | 11.3 | 6.6 | 72.2 | 9.8 | — | — |
| 10.12 | sonication | 347.54 | 57344 | 2.6 | 26 | 13.5 | 57.6 | | — | — |

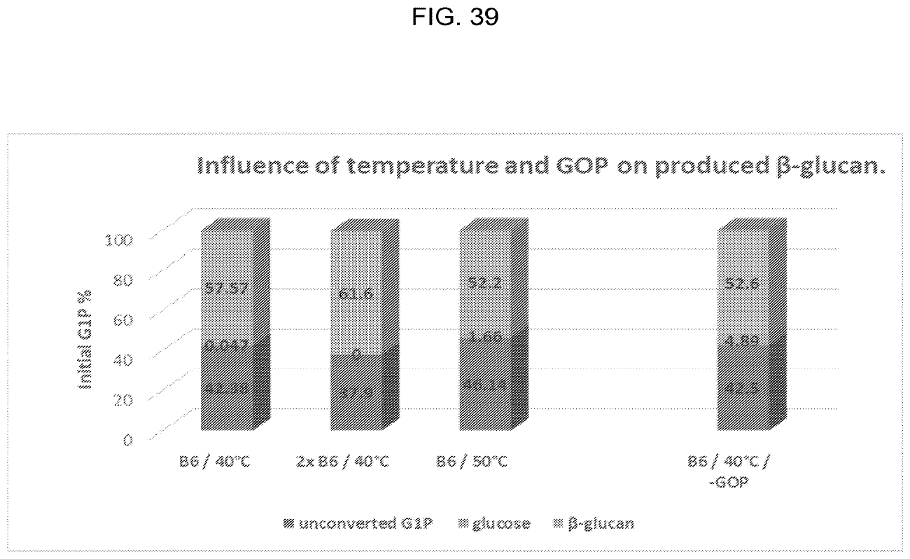

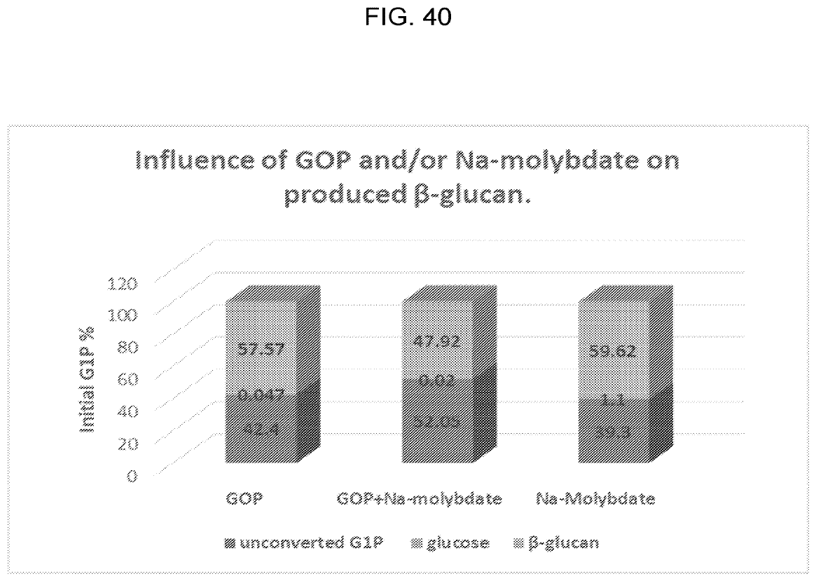

Results show that use of the SUMIZYME® GOP glucose oxidase and catalase cocktail reduces glucose formation and increases β-glucan yield (up to approximately 60%). β-glucans synthesized in reactions including the SUMIZYME® GOP glucose oxidase and catalase cocktail inhibitor had higher molecular weights (Trail 10.7) than equivalent reactions lacking the glucose oxidase and catalase cocktail (Trial 10.1). Similar results were seen with use of the sodium molybdate inhibitor (See FIG. 40 and Tables 30 and 31). In Trial 10.8, 3 U/mL of the B6 enzyme was added at the beginning of the reaction and the reaction was supplemented with an additional 3 U/mL B6 enzyme after 17 hours of incubation. This trail resulted in an approximately 2-4% increase in β-glucan yield. Reactions conducted at 50° C. (Trial 10.9) resulted in lower β-glucan yields and higher glucose amounts but the synthesized β-glucans had higher molecular weights. (See FIG. 39 and Table 31). While both the SUMIZYME® GOP glucose oxidase and catalase cocktail and the sodium molybdate inhibitors were added to Trail 10.10, the combination did not further improve β-glucan yield or increase the molecular weight of the resulting β-glucans.

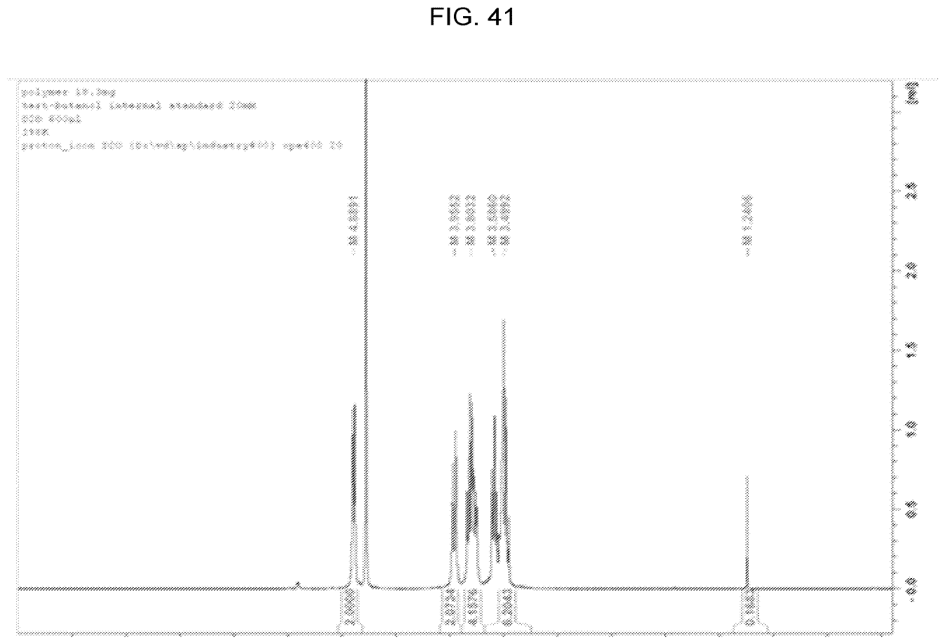

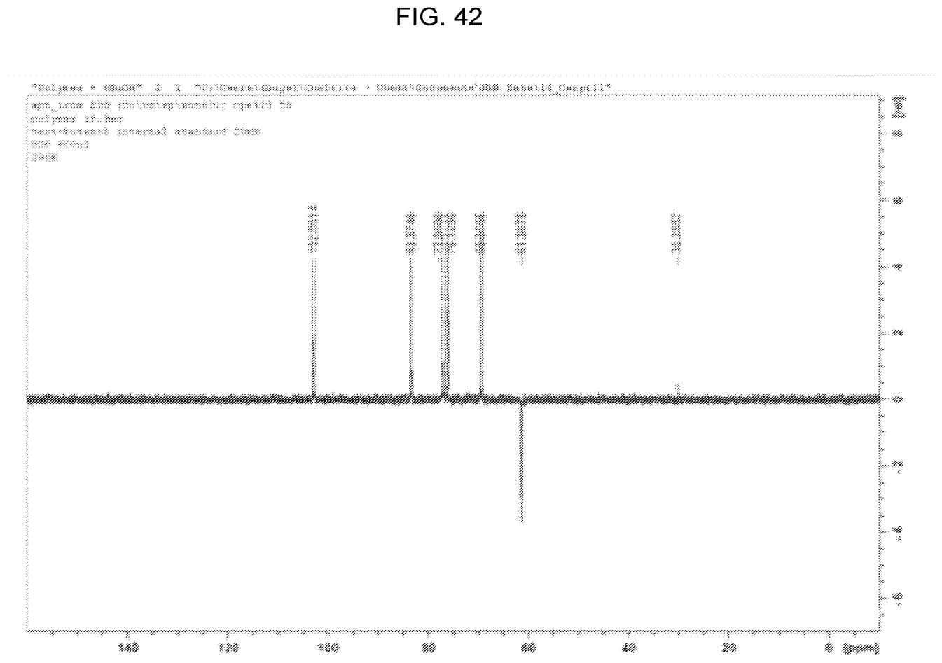

To confirm that the synthesized β-glucan is a linear β-1,2-oligoglucan, the isopropanol precipitation purified product from Trail 10.8 was freeze-dried for analysis using NMR. Structural identity of β-1,2-glucan was confirmed by 1H- and 13C-NMR. (FIGS. 41 and 42). The 1D 1H and 13C spectra were recorded on an Avance II Bruker spectrometer operating at 1H frequency of 400 MHz and equipped with a 5 mm 1H/BB BBO probe running Topspin 2.1 in the ICON environment. The sample temperature was set at 25° and controlled within ±0.1° C. with a Eurotherm 2000 VT controller. The sample was prepared from NMR by weighing 18.3 mg β-glucan and dissolving this in 598.86 μl of D2O and 1.14 μl of tBuOH as internal standard with a final concentration of 20 mM in a total volume of 600 μl. Following the addition of the solvent, the sample was vortexed and centrifuged multiple times. The resulting solution was transferred to a high-precision 5 mm NMR tube (Norell). Results of the 1D 1H- and 13C-NMR measurements were in agreement with the spectra obtained by Kakajima et al. (“1,2-β-Oligoglucan phosphorylase from Listeria innocua,” PLOS One, 9 (3), e92353, 2014).

### B6 βGP Concentration

To evaluate the effects of reduced enzyme concentration and enzyme activity, trails were run with homogenized crude enzyme lysate at concentrations of 3, 2, 1, or 0.5 U/mL reaction mixture. Additionally, the B6 enzyme used in these trails was from a separate growth and preparation of the E. coli cells to test the batch-to-batch reproducibility of the B6 enzyme activity. These reactions included the SUMIZYME® GOP glucose oxidase and catalase cocktail. Results are outlined in Tables 32 and 33.

TABLE 32

| | | Enzyme dosage | | | | | | | G1P | G1P |
|---|---|---|---|---|---|---|---|---|---|---|
| | | U enzyme/ml | | Initial | Residual | | | | conv to | conv to |
| | Temp | reaction | SUMIZYME ® | G1P | G1P | Glucose | Used G1P | Con v G1P | glucose | β-glucan |
| Trial | (° C.) | mixture | GOP | (mM) | (mM) | (mM) | (mM) | (mM) | (%) | (%) |
| 10.13 | 40 | 3 U/ml | + | 866 | 329.6 | 9.042 | 536.4 | 53.64 | 0.9 | 52.74 |
| 10.14 | 40 | 2 U/ml | + | 856 | 355.7 | 6.35 | 500.3 | 50.3 | 0.63 | 49.4 |
| 10.15 | 40 | 1 U/ml | + | 839 | 532.6 | 2.81 | 306.4 | 30.64 | 0.28 | 30.36 |
| 10.16 | 40 | 0.5/ml | + | 850 | 791.0 | 0.02 | 59.0 | 5.9 | 0.59 | 5.31 |
| 10.17 | 40 | 3 U/ml | ++ | 853 | 419.1 | 14.72 | 418.2 | 41.82 | 1.47 | 40.35 |

TABLE 33

| | Enzyme dosage | | | | | | | | | |
|---|---|---|---|---|---|---|---|---|---|---|
| | U enzyme/ml | GPC HMW system | | MW range (slicing area %) | | | | | | |
| | reaction | DP | MW | | 1000- | 5000- | 25000- | 20000- | 1000000- | |
| Trial | mixture | range | (kDa) | <1000 | 5000 | 25000 | 200000 | 1000000 | 5000000 | 5000000 |
| 10.13 | 3 U/ml | 444.5 | 73342 | 1.3 | 5.6 | 12.6 | 76 | 4.5 | — | — |
| 10.14 | 2 U/ml | 641.1 | 105776 | 1.1 | 5.5 | 5.2 | 75.9 | 12.3 | — | — |
| 10.15 | 1 U/ml | 826.7 | 136401 | 1.1 | 9.2 | 3.2 | 62.3 | 24.2 | — | — |
| 10.16 | 0.5/ml | 12.3 | 2024 | 3.2 | 95.8 | 1 | — | — | — | — |
| 10.17 | 3 U/ml | 474.27 | 78254 | 1 | 5.4 | 10.7 | 77.6 | 5.3 | — | — |

Comparing Trials 10.17 and 10.13 confirms the reproducibility of the B6 enzyme between preparations. While the molecular wight of the synthesized β-glucans was higher at lower enzyme concentrations, overall β-glucan yield was highest using the 3 U/ml concentration. In Trial 10.17, both the G1P substrate and the B6 enzyme were separately pre-incubated with SUMIZYME® GOP glucose oxidase and catalase cocktail, however this additional step did not further improve β-glucan yield.

### Large-Scale β-Glucan Synthesis Using B6 βGP

Five large-scale trials were run using a starting concentration of 100 g G1P, 3 U/ml pre-incubated B6 enzyme, 15 mg (per ml B6 enzyme) SUMIZYME® GOP glucose oxidase and catalase cocktail, and 1M DTT in 100 mM phosphate buffer pH 7. The B6 enzyme was pre-incubated with the SUMIZYME® GOP glucose oxidase and catalase cocktail for 5 hours at room temperature at a pH between 6.5-7. Each reaction was run at 40° C. for 24 hours. Following the reaction, the reaction mixture was heated at 90° C. for 10 minutes to deactivate the B6 enzyme. Results are outlined in Tables 34 and 35.

The β-glucan products for all five trials were separated from the reaction mixture by ethanol precipitation, dried, and pooled. Approximately 120 g of dried samples was used for testing in Example 11.

TABLE 34

| | | | HPLC fingerprint Ag | | | G1P | Glucose | | | G1P | G1P |
|---|---|---|---|---|---|---|---|---|---|---|---|
| | | | col | | Initial | after | after | Used | Conv | conv to | conv to |
| | Temp | SUMIZYME ® | Dpn | Dextrose | G1P | 24 h | 24 h | G1P | G1P | glucose | β-glucan |
| Trial | (° C.) | GOP | (%) | (%) | (mM) | (mM) | (mM) | (mM) | (%) | (%) | (%) |
| 10.18 | 40 | + | 93.28 | 6.44 | 1000 | 443.1 | 17.503 | 556.9 | 55.69 | 1.7 | 53.94 |
| 10.19 | 40 | + | 97.01 | 2.99 | 924 | 376.2 | 16.197 | 547.8 | 54.78 | 1.62 | 54.62 |
| 10.20 | 40 | + | 95.54 | 4.46 | 924 | 338.6 | 9.373 | 585.4 | 58.54 | 0.94 | 58.54 |
| 10.21 & | 40 | + | 98.7 | 1.3 | 924 | 368.9 | 17.826 | 555.1 | 55.51 | 1.78 | 53.73 |
| 10.22 | | | | | | | | | | | |
| Average | | | 96.6 | 3.3 | | | | | | 1.7 | 56.0 |

TABLE 35

| | GPC HMW system | | MW range (slicing area %) | | | | | | |
|---|---|---|---|---|---|---|---|---|---|
| | DP range | MW (kDa) | | 1000- | 5000- | 25000- | 200000- | 1000000- | |
| Trial | overall | overall | <1000 | 5000 | 25000 | 200000 | 1000000 | 5000000 | >5000000 |
| 10.18 | 402.1 | 66.353 | 2.9 | 10.3 | 16.8 | 65.3 | 4.5 | 0.1 | — |
| 10.19 | 306.1 | 50.502 | 0.4 | 5.6 | 24.2 | 68.8 | 1.1 | — | — |
| 10.20 | 437.3 | 72.157 | 1.3 | 5.6 | 13.3 | 75.7 | 4.1 | — | — |
| 10.21&10.22 | 430.1 | 70.963 | 0 | 2 | 14.7 | 80 | 3.3 | — | — |
| Average | 421 | 69.436 | 0 | 3.7 | 16 | 76.7 | 3.6 | — | — |

The reaction solution is cooled down to 37° C. and the pH is adjusted from 7.23 to 4.8 by adding HCl 19.2%. Conversion of the unreacted GP into glucose occurred by performing an additional incubation with 0.5 g acid phosphatase at 37° C. for 24 hours. At regular time intervals, the phosphate content is measured until a constant value is obtained. Constant phosphate measurements were used as an indicator of reaction completion when no additional phosphate is released. After heating the reaction mixture at 70° C. for 10 minutes, the solution is filtrated over a buchner with paper filter. The presence of a kind of precipitate or wisps after washing with 50 ml demi-water and which represents only 0.1% (dried product) of the total G1P.

In total, 400 ml filtrated reaction solution is recuperated with a conductivity of 54.6 mS/cm. Four Spectra/Por® Dialysis membrane Standard RC tubings MWCO 6-8 kD are filled each with 100 ml reaction mixture. A 24-hour dialysis against tap water (flowing) is executed, measuring the conductivity, phosphate and glucose (stick method). Last dialysis occurs in milli Q water, followed by concentration at the rotary evaporator to about 50 ml or 50% ds, measured on the IR balance.

### Example 11—Characterization of β-1,2-Oligoglucan Synthesized by B6 βGP

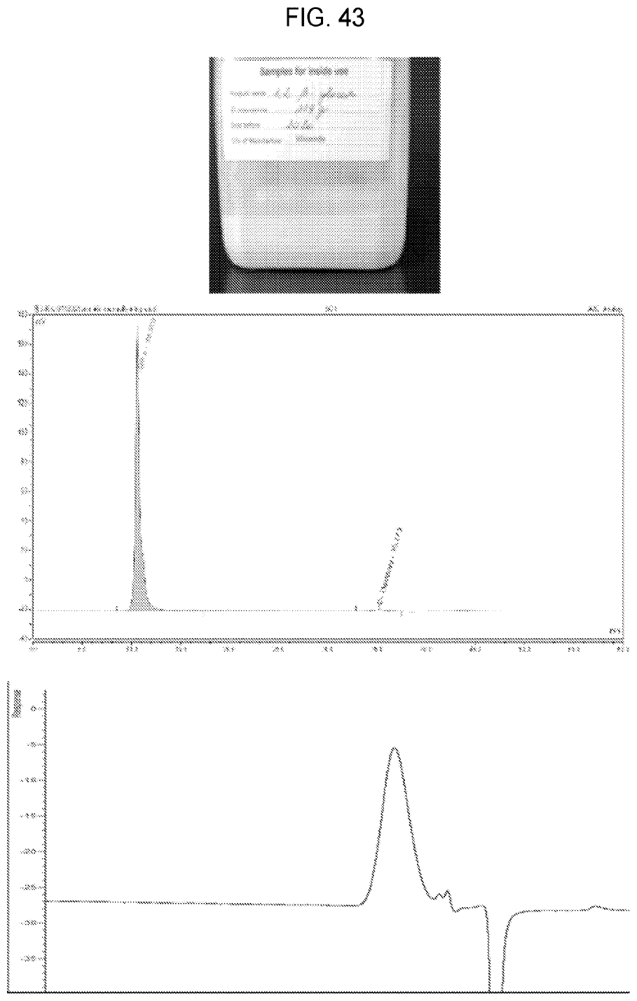

The pooled β-glucan samples obtained from Trails 10.18-10.22 of Example 10 were further analysed. (FIG. 43). Table 36 outlines the collective molecular weight distribution of the combined β-glucan product.

TABLE 36

| G1P conv to β-glucan (%) | | 56 |
|---|---|---|
| G1P conv to glucose (%) | | 1.7 |
| HPLC fingerprint Ag+ | DPN (%) | 98.5 |
| | dextrose (%) | 1.5 |
| GPC HMW system | DP range (Mn/165) | 157.3 |
| | MW (kDa) | 69.436 |
| MW range (slicing area %) | <1000 | — |
| | 1000-5000 | 3.7 |
| | 5000-25000 | 16 |
| | 25000-200000 | 76.7 |
| | 200000-1000000 | 3.6 |
| | 1000000-5000000 | — |
| | >5000000 | — |

Across Trials 10.18-10.22, an average of 56% G1P is converted to β-glucan together with conversion to glucose of only 1.7%. The β-glucan had a purity of 98.5% and a DP of 157. 76% of the MW is situated in the MW range of 2500 to 200000 kDa.

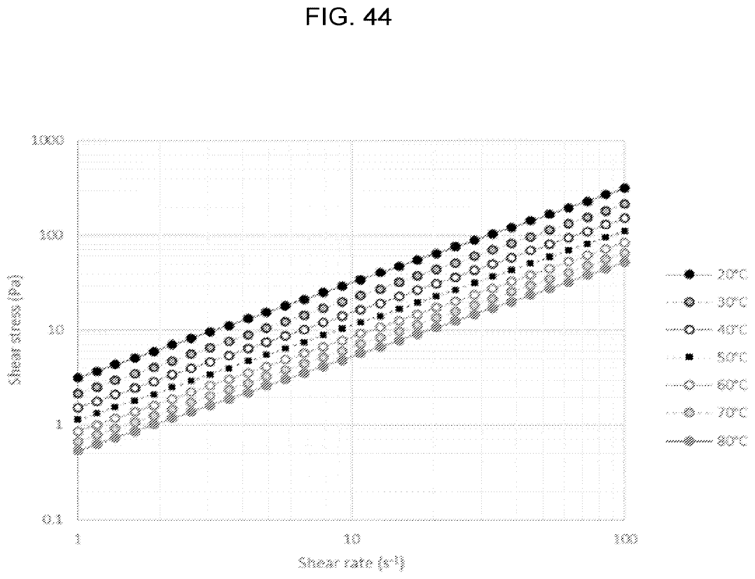

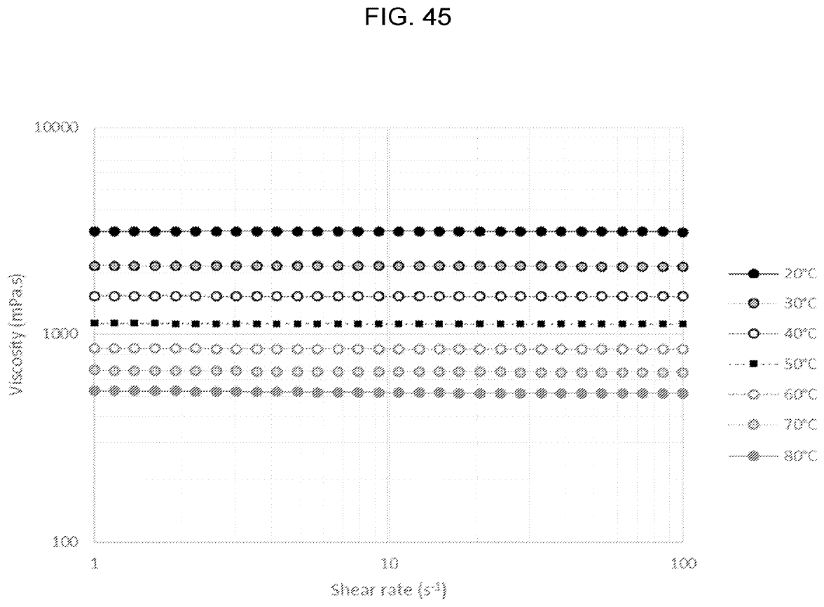

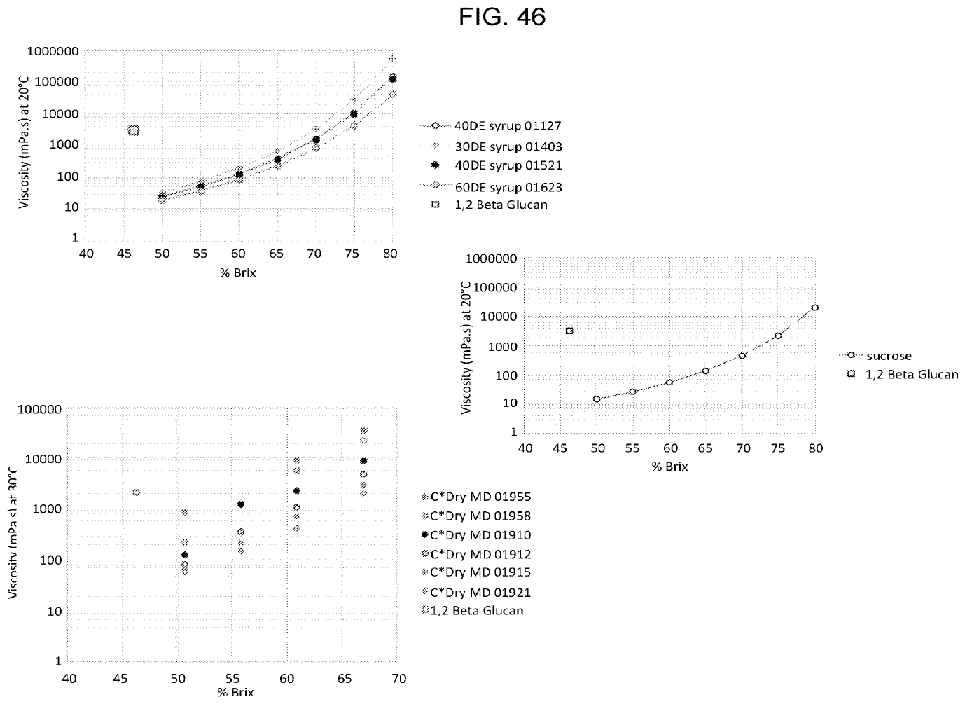

The viscosity and shear thinking characteristics of the synthesized β-glucan were also analysed. A β-glucan syrup was prepared with 50% dry substance (prepared from samples make in Trails 10.18-10.22) with 20 g powder and 20 g low conductivity water. The solution was stirred and equilibrated at 60° C. until the solution obtained optical clarity. Prior to analysis the samples were stored at 20° C. The isothermal viscosity was measured at 20° C. (reference temperature) and at a set of temperatures in the range of 30-80° C. (in duplicate; FIGS. 44 and 45). The cooling water-bath was set at 15° C. As shown in FIG. 46, the viscosity and shear thinning of the produced β-glucan is higher than sucrose or commercially available maltodextrins.

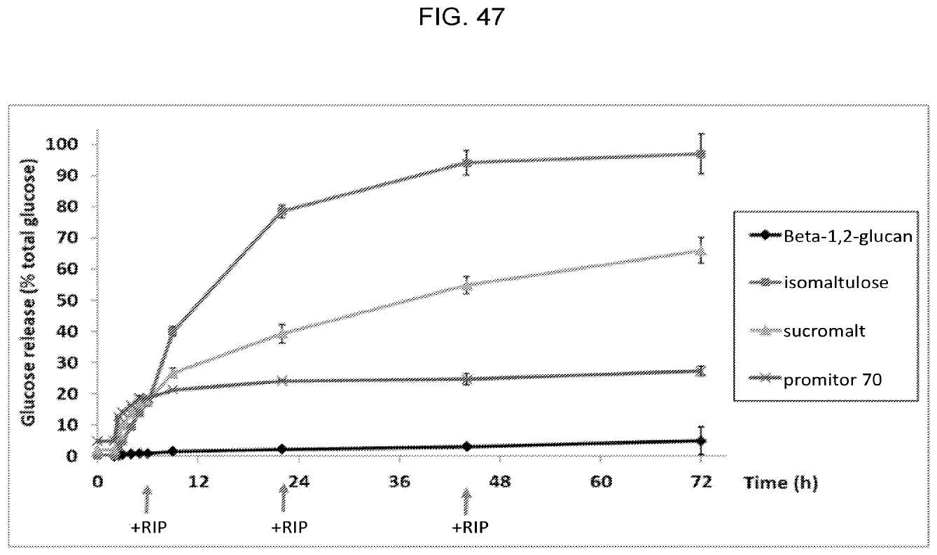

Digestibility of the β-glucan product was assessed using an in vitro digestion assay and compared to the digestibility of sucromalt, isomaltulose, and Promitor 70 soluble corn fiber. The samples were subjected to in vitro digestion, in triplicate, over 72 hours and analyzed for glucose release using a glucose oxidase colorimetric assay. As demonstrated in FIG. 47, the β-glucan is indigestible with minimal glucose release over the 72-hour incubation. Glucose release (as % total glucose) after 72 hours was 5% from the β-1,2-glucan, 97% from isomaltulose, 66% from sucromalt, and 27% from Promitor 70. These results provide encouraging evidence that the digestibility of beta-1,2-glucan is likely very limited. The 5% glucose release from the β-glucan sample is likely due to residual glucose being autohydrolyzed from the rat intestinal powder (RIP) at the time points indicated in FIG. 47.

### Example 12—β-Glucan Synthesis Starting from G1P and Pure B6 with Glucose as Primer

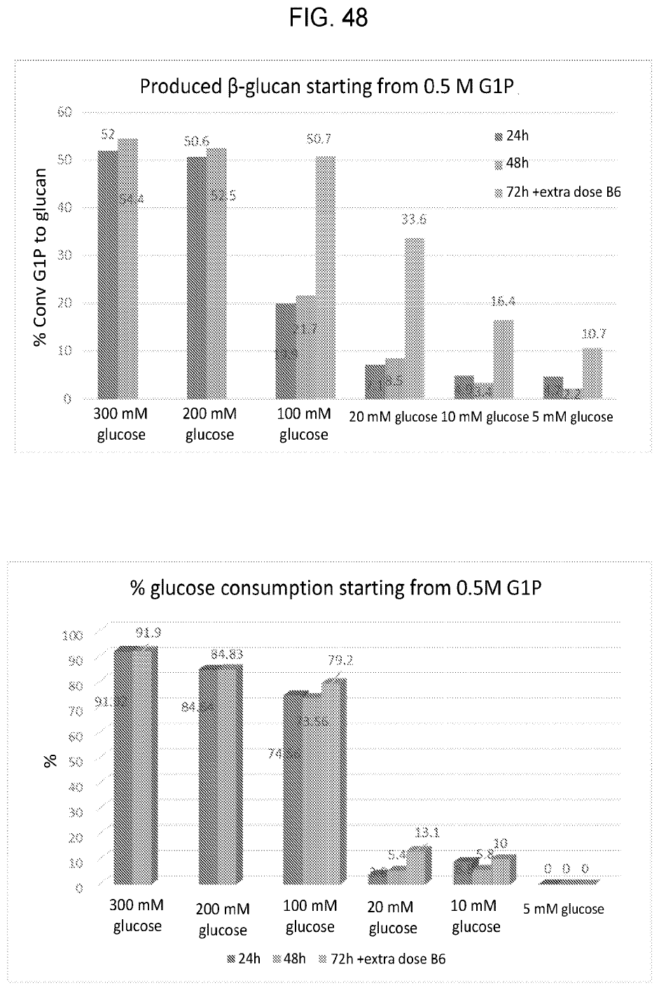

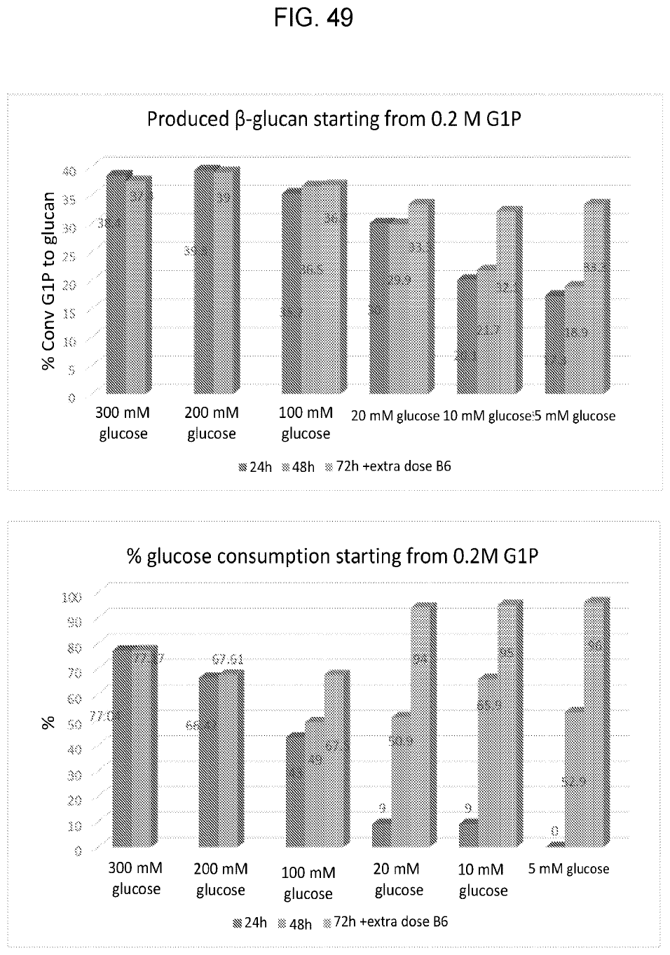

While previous reactions with run with a crude cell lysate containing the B6 enzyme, trails in this example use His-tag purified B6 enzyme. Reactions used a starting concentration of either 0.2M or 0.5M G1P and 5, 10, 20, 100, 200, or 300 mM glucose as a primer. No DTT or SUMIZYME® GOP glucose oxidase and catalase cocktail were used in these reactions. Reactions were run in 100 mM phosphate buffer pH 7. 0.32 ml (at 5.2 mg/ml and 140 U/ml) purified B6 enzyme was added to the reaction mixture. This results in addition of the B6 enzyme at the same 3U/ml reaction mixture that was run in previous trials. Reactions were run at 40° C. for 24, 48, and 72 hours followed by enzyme deactivation at 90° C. for 10 minutes. Results are outlined in FIGS. 48 and 49 and Table 37.

TABLE 37

| G1P | 0.5M | | 0.5M | | 0.5M | | 0.5M | | 0.5M | | 0.5 M | | 0.2M | |
|---|---|---|---|---|---|---|---|---|---|---|---|---|---|---|
| Glucose | 300 | mM | 200 | mM | 100 | mM | 20 | mM | 10 | mM | 5 | mM | 300 | mM |
| Time | 48 | h | 48 | h | 72 | h | 72 | h | 72 | h | 72 | h | 48 | h |

| Mn | 491.5 | 6688 | 30234 | 25786 | 15571 | 13490 | 4826 |
|---|---|---|---|---|---|---|---|
| Mw | 21723 | 38552 | 165126 | 353550 | 441585 | 496699 | 12999 |
| Polydispersity | 4.4 | 5.8 | 5.5 | 13.7 | 28.4 | 36.8 | 2.7 |
| MW range | Slicing area % | | | | | | |
| <1000 | 1.6 | 0.9 | 0.2 | 0.2 | 0.4 | 0.4 | 0.9 |
| 1000-5000 | 32.7 | 22.9 | 3.4 | 5.2 | 8.2 | 11.0 | 36.3 |
| 5000-25000 | 40.7 | 40.2 | 5.6 | 1.9 | 2.5 | 2.0 | 50.2 |
| 25000-200000 | 24.8 | 34.2 | 59.1 | 22.7 | 16.2 | 14.5 | 12.6 |
| 200000-1000000 | 0.2 | 1.9 | 31.3 | 66.5 | 65.0 | 61.8 | 0.0 |
| 1000000-5000000 | 0.0 | 0.0 | 0.4 | 3.5 | 7.8 | 10.2 | 0.0 |
| >5000000 | 0.0 | 0.0 | 0.0 | 0.0 | 0.0 | 0.2 | 0.0 |

| G1P | 0.2M | | 0.2M | | 0.2M | | 0.2M | | 0.2M | |
|---|---|---|---|---|---|---|---|---|---|---|
| Glucose | 200 | mM | 100 | mM | 20 | mM | 10 | mM | 5 | mM |
| Time | 48 | h | 72 | h | 72 | h | 72 | h | 72 | h |

| Mn | 5702 | 12909 | 21934 | 27374 | 31430 |
|---|---|---|---|---|---|
| Mw | 20625 | 99724 | 244429 | 364025 | 473581 |
| Polydispersity | 3.6 | 7.7 | 11.1 | 13.3 | 15.1 |
| MW range | Slicing area % | | | | |
| <1000 | 1.0 | 0.5 | 0.3 | 0.2 | 0.2 |
| 1000-5000 | 27.7 | 9.1 | 5.9 | 5.0 | 4.4 |
| 5000-25000 | 40.7 | 19.2 | 4.7 | 2.5 | 2.0 |
| 25000-200000 | 21.5 | 60.7 | 32.3 | 19.7 | 10.7 |
| 200000-1000000 | 0.2 | 10.3 | 56.8 | 71.8 | 78.3 |
| 1000000-5000000 | 0.0 | 0.1 | 0.1 | 0.8 | 4.4 |
| >5000000 | 0.0 | 0.1 | 0.0 | 0.1 | 0.0 |

For reactions starting with 0.5 M G1P, lower concentrations of glucose (e.g., 20 mM) were associated with significantly lower β-glucan synthesis and glucose consumption, while higher initial glucose concentrations (e.g., 100-300 mM) were associated with high glucose consumption (up to about 91%, FIG. 48). However, for reactions starting with 0.2 M G1P, glucose consumption at all initial glucose concentrations was at least about 50% at 48 hours (FIG. 49).

The data collected for the 0.5 M G1P reactions suggest that the higher the initial glucose concentration, the faster the reaction reaches its equilibrium. For the 300, 200, and 100 mM initial glucose concentrations at 0.5 M G1P, the total G1P concentration decreased until approximately 52% of the initial G1P was concerted to β-glucans between 48 and 72 hours. Given that there is no marked increase in glucose concentration over time (i.e., above the initial glucose primer concentration), there are no side reactions cleaving G1P to glucose in the presence of pure B6. As shown in FIG. 49, the initial 0.2 M G1P concentration was converted to an equilibrium of around 35% β-glucans while there was only a consumption of around 70% of the initial supplied glucose. This suggests that once a molecule of glucose is used as primer by B6 and converted to DP 2, this disaccharide becomes the preferential acceptor for the enzyme to continue β-glucan synthesis.

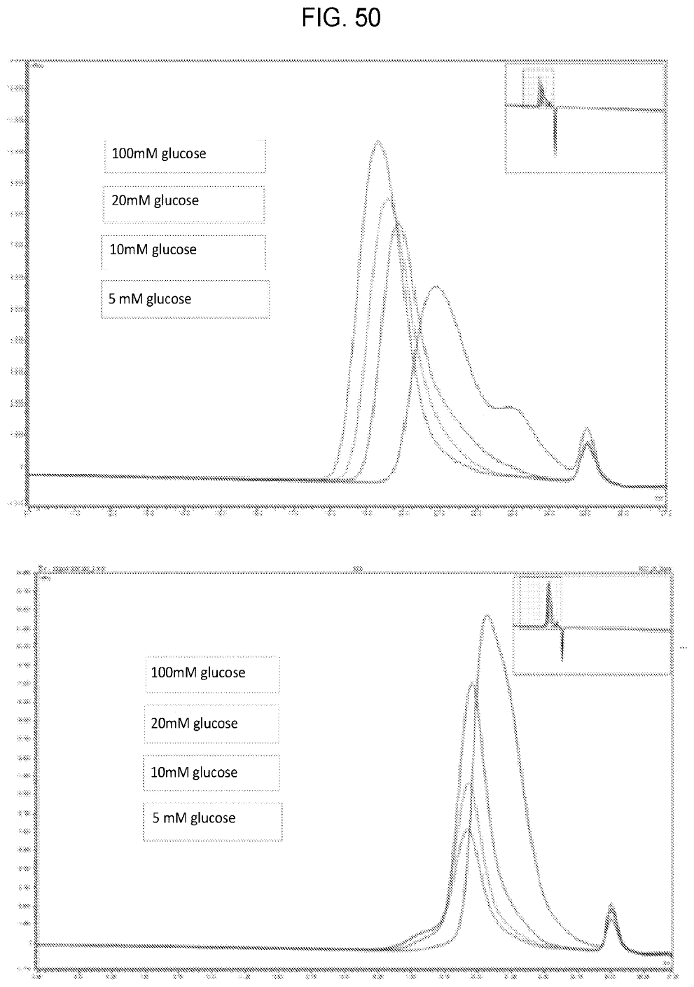

GPC analysis showed that the weight-average molecular weight (MW) of the synthesized β-glucan depends on the concentration of glucose added as a primer (See Table 37 and FIG. 50). The largest polymers (DP=2700) are produced with the lowest concentration of glucose as primer (10 mM). These reactions with pure enzyme resulted in longer β-glucans than the reactions performed with crude enzyme and the β-glucan size is correlated to primer concentration.

### Example 12—Synthesis of β-Glucan from Maltodextrin

This example demonstrates the overall process for the production of β-glucans from maltodextrin via a G1P intermediate. In this example two enzymatic processes are performed in the same reaction vessel to test if the equilibrium of the first reaction could be shifted to the right (i.e., towards production of G1P) when G1P was consumed in the second reaction.

This one-pot approach requires both enzymes to be active at the same pH and temperature. The reactions were run at 40° C. and pH 7. Given that the B6 βGP enzyme has higher activity at 40° C. than the A8 αGP enzyme, the concentration of the A8 enzyme in the reaction was increased to account for the difference in activity. Enzyme ration, phosphate concentration, maltodextrin type and concentration, glucose concentration, and incubation time were all variables to in this two enzyme β-glucan synthesis.

Reaction with MDX 01912 with Crude A8 and B6

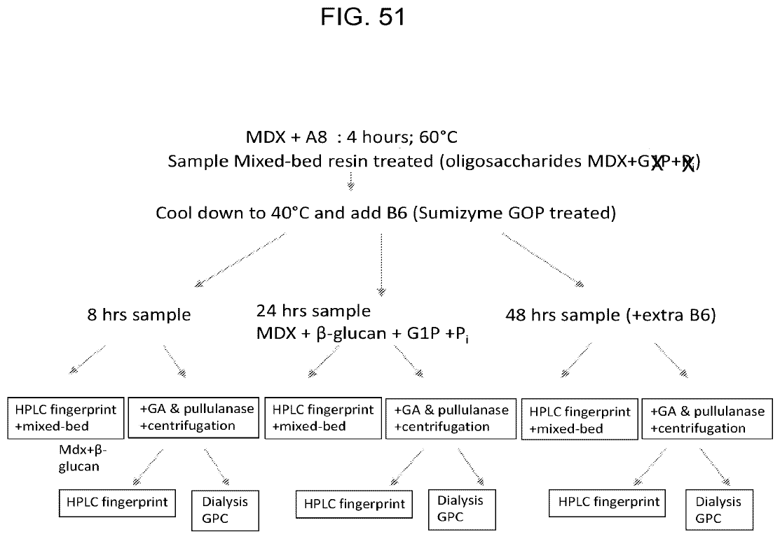

Prior to the reaction, maltodextrin 01912 was debranched by incubation with 0.1% iso-amylase and 0.2% pullulanase at pH 4.8 and 50° C. for 5 hours. 128U of the A8 αGP enzyme (10 ml at approximately 12-13 U/ml) was added to the pH 7 reaction mixture containing debranched maltodextrin 01912 (at 6.5% dry substrances) and Na2HPO4 and incubated at 60° C. for 4 hours to generate G1P prior to addition of the B6 βGP enzyme. Approximately 21.1% G1P (maltodextrin conversation percentage) was produced during this incubation. After cooling to 40° C., 18U (0.43 mL at 42 U/ml) of the B6 enzyme, which had been pre-treated with the SUMIZYME® GOP glucose oxidase and catalase cocktail, was added and the reaction was maintained at 40° C. Samples were taken at 8 and 24 hours for analysis. After 24 hours, a supplemental addition of 18U of B6 was added to the reaction and a final sample was taken at 48 hours for analysis. Prior to analysis, samples were treated with a combination of glucoamylase and pullulanase to hydrolyze any remaining maltodextrin and the samples were passed over a mixed-bed resin to remove G1P and phosphate from the reaction. Samples were then characterized by high molecular weight GPC and HPLC fingerprinting (Ag+ column). Reaction schemes are shown in FIG. 51 and the results are outlined in Tables 38 and 39.

TABLE 38

| | Trial | | | | | | | | | | | | | | | |
|---|---|---|---|---|---|---|---|---|---|---|---|---|---|---|---|---|
| | Area % | | | | | | | | | | | | | | | |
| Sample code | DPn | DP13 | DP12 | DP11 | DP10 | DP9 | DP8 | DP7 | DP6 | DP5 | DP4 | DP3 | DP2 | Dextrose | Fructose | Unknown1 |
| Debranched | 30.2 | 2.84 | 3.89 | 3.31 | 4.06 | 4.6 | 5.74 | 7.79 | 9 | 7.45 | 6.69 | 8.57 | 5.3 | — | — | 1.57 |
| MDX 01912 | | | | | | | | | | | | | | | | |
| MDX +A8 | 31.46 | 1.18 | 1.19 | 1.87 | 2.18 | 2.82 | 3.89 | 5.36 | 7.10 | 10.16 | 11.56 | 9.72 | 6.02 | 4.85 | 0.09 | 0.55 |
| (4 hrs) | | | | | | | | | | | | | | | | |
| +B6 mix-bed | 33.13 | 1.10 | 1.89 | 2.24 | 2.74 | 3.30 | 4.25 | 5.35 | 6.85 | 8.74 | 11.00 | 9.07 | 5.79 | 3.65 | — | 0.90 |
| (8 h) | | | | | | | | | | | | | | | | |
| +B6 +GA | 12.92 | — | — | — | — | — | — | — | 0.12 | 0.23 | 0.23 | 0.20 | 1.00 | 83.24 | — | 2.06 |
| (8 h) | | | | | | | | | | | | | | | | |
| +B6 mix-bed | 35.70 | 1.45 | 1.45 | 2.09 | 2.39 | 2.90 | 3.72 | 5.05 | 6.95 | 9.84 | 12.21 | 7.98 | 5.06 | 2.77 | — | 0.44 |
| (24 h) | | | | | | | | | | | | | | | | |
| +B6 +GA | 10.70 | — | — | — | — | — | — | — | 0.10 | 0.13 | 0.22 | 0.18 | 1.14 | 85.56 | — | 1.97 |
| (24 h) | | | | | | | | | | | | | | | | |
| +B6 mix-bed | 21.83 | 0.85 | 1.27 | 1.05 | 1.20 | 1.14 | 1.25 | 1.65 | 2.82 | 5.57 | 9.27 | 13.48 | 15.53 | 21.08 | 0.76 | 1.25 |
| (48 h) | | | | | | | | | | | | | | | | |
| +B6 +GA | 15.58 | — | — | — | — | — | — | — | — | — | 0.15 | 0.16 | 1.76 | 82.35 | — | — |
| (48 h) | | | | | | | | | | | | | | | | |

TABLE 39

| | Debranched | | | |
|---|---|---|---|---|
| | MDX 01912 | 8 hrs | 24 hrs | 48 hrs |
| Mn | 1076 | 1290 | 2221 | 2354 |
| Mw | 3847 | 2029 | 3723 | 3151 |
| Polydispersity | 3.6 | 1.6 | 1.7 | 1.3 |

| | |
|---|---|
| Mw range | Slicing (Area %) |

| | | | | |
|---|---|---|---|---|
| <1000 | 35.8 | 17.3 | 5.1 | 2.9 |
| 1000-5000 | 47.7 | 80.5 | 87.9 | 92.4 |
| 5000-25000 | 14.5 | 2.3 | 5.7 | 4.1 |
| 25000-200000 | 2.0 | — | 1.4 | 0.6 |
| 200000-1000000 | — | — | — | — |
| 1000000-5000000 | — | — | — | — |
| >5000000 | — | — | — | — |

Reaction with Zulkowsky Starch or Low Viscosity Branched Dextrin with HIS-Tag Purified A8 and B6

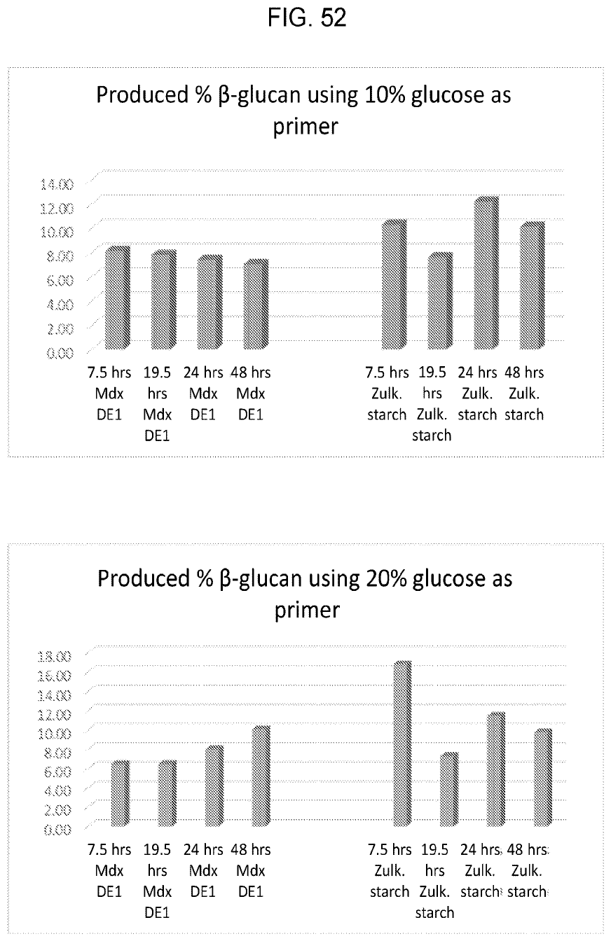

Prior to reaction, Zulkowsky and maltodextrin DE1 were separately debranched by incubation with 0.1% iso-amylase and 0.2% pullulanase at pH 4.8 and 50° C. for 5 hours. Four separate reactions were run containing (i) 5.2% MDX DE1 and 1.3% glucose; (ii) 5.2% Zulkowsky starch and 1.3% glucose; (iii) 5.85% MDX DE1 and 0.65% glucose; or (iv) 5.85% Zulkowsky starch and 0.65% glucose. Each reaction contained 6.5% dry substances and an equimolar amount of KH₂PO₄. 128U of the His-tag purified A8 αGP enzyme (21 ml at approximately 6 U/ml) was added to each of the pH 7 reaction mixtures and incubated at 60° C. for 4 hours to generate G1P prior to addition of the B6 βGP enzyme. After cooling to 40° C., 18U (0.12 mL at approximately 160 U/ml) of the His-tag purified B6 enzyme was added and the reaction was maintained at 40° C. Samples were taken at 7.5, 19.5, 24, and 48 hours for analysis. After 24 hours, a supplemental addition of 18U of B6 was added to the reaction. Prior to analysis, samples were treated with a combination of glucoamylase and pullulanase to hydrolyze any remaining maltodextrin and the samples were passed over a mixed-bed resin to remove G1P and phosphate from the reaction. Samples were then characterized by high molecular weight GPC and HPLC fingerprinting (Ag+ column). The results are outlined in Tables 40 and 41 and FIG. 52.

Reaction with Zulkowsky Starch Using Immobilized HIS-Tag Purified A8 and B6

Duolite A-568 carrier immobilized His-tag purified A8 and B6 enzymes were loaded onto separate glass jacketed columns (2 cm×14 cm) in series to separate the A8 catalyzed synthesis of G1P and the B6 catalyzed synthesis of β-glucan. A reaction temperature of 50° C. was maintained in both columns using a circulating water back. The pH 7 substrate solution with 7% dry substances and containing 3.2% debranched Zulkowsky starch, 0.8% glucose (as a primer), and 3.2% KH₂PO₄ was circulated through the column at 3 BV per hour using a peristaltic pump. Samples were taken after 7, 28, and 52 hours. Prior to oligosaccharides analysis, samples were incubated overnight with 0.2% glucoamylase and 0.1% pullulanase at 50° C., pH 4.5. Results are outlined in Tables 42 and 43.

TABLE 40

| | Zulkowsky starch + 10% glucose | | | | Zulkowsky starch + 20% glucose | | | |
|---|---|---|---|---|---|---|---|---|
| | 7.5 hrs | 19.5 hrs | 24 hrs | 48 hrs | 7.5 hrs | 19.5 hrs | 24 hrs | 48 hrs |
| Mn | 2283 | 2146 | 1810 | 1738 | 2222 | 1434 | 1449 | 1971 |
| Mw | 3732 | 3228 | 2677 | 2814 | 3553 | 2586 | 2794 | 3323 |
| Polydispersity | 1.6 | 1.5 | 1.5 | 1.6 | 1.6 | 1.8 | 1.9 | 1.7 |

| MW range | Slicing (Area %) | | | | Slicing (Area %) | | | |
|---|---|---|---|---|---|---|---|---|
| <1000 | 6.18 | 7.96 | 10.72 | 5.97 | 4.76 | 16.19 | 16.88 | 10.98 |
| 1000-5000 | 76.16 | 77.98 | 81.49 | 79.32 | 56.02 | 74.99 | 72.51 | 79.54 |
| 5000-25000 | 17.10 | 14.06 | 7.79 | 14.71 | 38.96 | 8.81 | 10.61 | 9.48 |
| 25000-200000 | 0.56 | | | | 0.26 | | | |
| 200000-1000000 | | | | | | | | |
| 1000000-5000000 | | | | | | | | |
| >5000000 | | | | | | | | |

TABLE 41

| | MDX DE1 + 10% glucose | | | | MDX DE1 + 20% glucose | | | |
|---|---|---|---|---|---|---|---|---|
| | 7.5 hrs | 19.5 hrs | 24 hrs | 48 hrs | 7.5 hrs | 19.5 hrs | 24 hrs | 48 hrs |
| Mn | 2549 | 2183 | 2375 | 1988 | 2711 | 2335 | 2281 | 2091 |
| Mw | 3954 | 3444 | 3728 | 3721 | 4985 | 4266 | 3387 | 3512 |
| Polydispersity | 1.6 | 1.6 | 1.6 | 1.9 | 1.8 | 1.8 | 1.5 | 1.7 |

| MW range | Slicing (Area %) | | | | Slicing (Area %) | | | |
|---|---|---|---|---|---|---|---|---|
| <1000 | 4.19 | 7.27 | 5.29 | 8.69 | 4.76 | 6.46 | 8.27 | 7.70 |
| 1000-5000 | 68.79 | 71.78 | 72.78 | 66.63 | 56.02 | 63.99 | 73.16 | 70.15 |
| 5000-25000 | 26.98 | 20.95 | 21.93 | 24.68 | 38.96 | 29.55 | 18.48 | 22.15 |
| 25000-200000 | 0.04 | | | | 0.26 | | 0.09 | |
| 200000-1000000 | | | | | | | | |
| 1000000-5000000 | | | | | | | | |
| >5000000 | | | | | | | | |

TABLE 42

| | Rel. Area % | | | | | | | | | | | | | |
|---|---|---|---|---|---|---|---|---|---|---|---|---|---|---|
| | | | | | | | | | | | | | Total | Total DP |
| Samples | DPn | DP9 | DP8 | DP7 | DP6 | DP5 | DP4 | DP3 | DP2 | Dextrose | Fructose | Unknown1 | DP* | unknown** |
| Zulkowsky starch | 90.02 | 1.87 | 1.42 | 1.41 | 1.48 | 0.97 | 0.82 | 0.79 | 0.33 | 0.9 | n.d. | n.d. | | |
| debranched | | | | | | | | | | | | | | |
| Start substrate | 81.1 | 0.68 | 0.64 | 0.59 | 0.57 | 0.37 | 0.28 | 0.28 | 0.1 | 15.4 | n.d | n.d | 84.61 | |
| (Starch + glucose + | | | | | | | | | | | | | | |
| KH₂PO₄) | | | | | | | | | | | | | | |
| β-glucan synthesis | 15.98 | 0.61 | 0.56 | 0.7 | 0.73 | 0.96 | 1.04 | 4.59 | 6.35 | 65.23 | n.d. | | | |
| after 7 hrs | | | | | | | | | | | | | | |
| β-glucan synthesis | 16.07 | 0.76 | 0.83 | 0.94 | 1.16 | 1.28 | 1.18 | 5.17 | 4.47 | 64.52 | n.d. | 3.61 | 31.86 | 33.05 |
| after 28 hrs | | | | | | | | | | | | | | |
| β-glucan synthesis | 26.48 | n.d. | 1.25 | 1.3 | 1.37 | 1.3 | 0.66 | 0.79 | 11.65 | 47.11 | n.d. | 7.09 | 44.8 | 48.2 |
| after 52 hrs | | | | | | | | | | | | | | |

*Total DP is the sum of DPn through DP2
**Total DP unknown is the total DP considering impurities from enzymes and ions still present in the reaction mixture

TABLE 43

| | Debranched | After 7 hours | After 52 hours |
|---|---|---|---|
| | Zulkowsky starch | recirculation | recirculation |
| Mn | 3457 | 873 | 1231 |
| Mw | 8901 | 1758 | 2352 |
| Polydispersity | 2.6 | 2.013 | 1.91 |
| MW range | Slicing (Area %) | | |
| <1000 | 4.42 | 47.83 | 20.8 |
| 1000-5000 | 36.48 | 46.05 | 72.44 |
| 5000-25000 | 53.94 | 6.12 | 6.76 |
| 25000-200000 | 5.15 | — | — |
| 200000-1000000 | 0.01 | — | — |
| 1000000-5000000 | — | — | — |
| >5000000 | — | — | — |

### Example 13—β-Glucan Viscosity

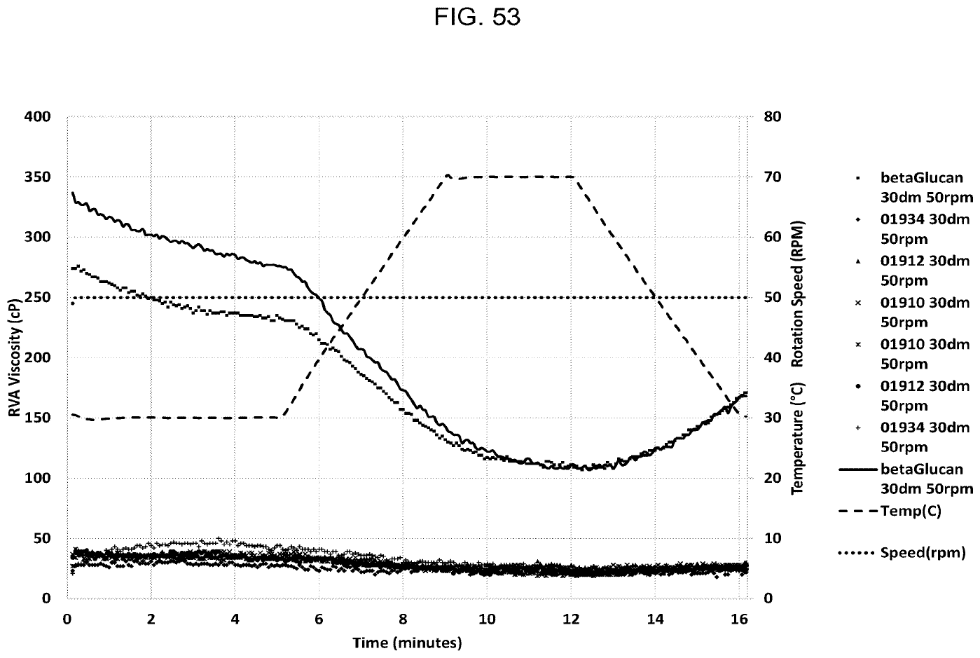

Viscosity of a pure 30% ds DP 150 β-glucan sample (produced as described in Example 10) was measured as a function of temperature using a rapid visco analyzer with stirring at 50 rpm. For comparison, viscosity of various maltodextrin samples was also measured. As reported in FIG. 53, the DP 150 β-1,2-oligoglucan had significantly higher viscosity at all temperatures than any of the maltodextrin samples assayed.

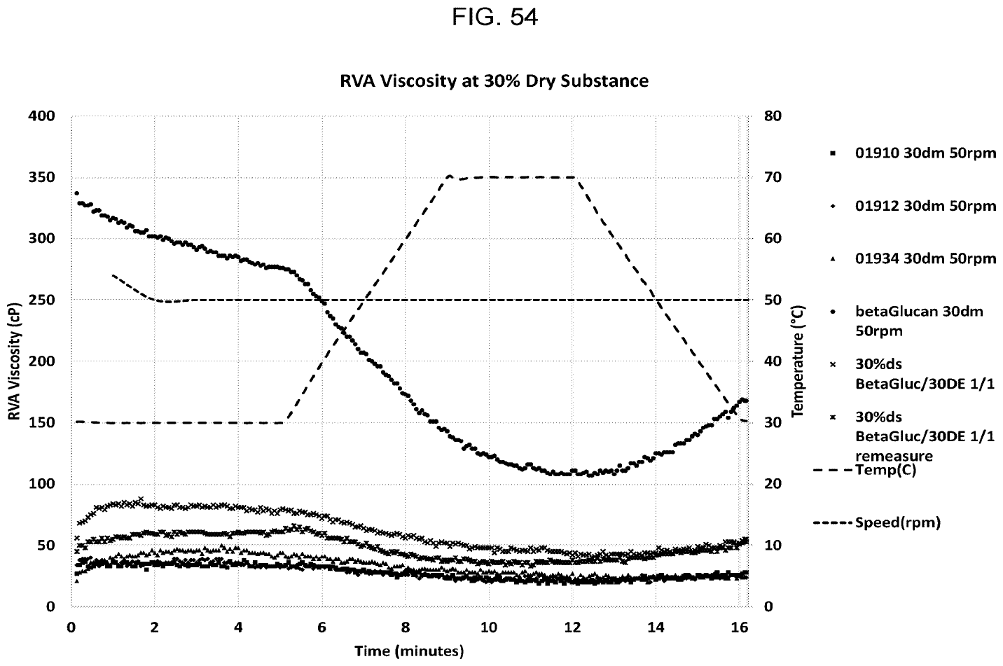

Similarly, viscosity of the β-glucan samples was compared to a mixture of 50% 30 DE syrup and 50% DP 150 β-glucan in addition of the various maltodextrin samples. (See FIG. 54) While the mixture has a slightly higher viscosity than the maltodextrin samples, the pure β-glucan sample has higher viscosity than all samples tested. The 50/50 mixture sample mimics the β-glucan composition immediately following synthesis by the B6 enzymes and prior to purification. This sample represents the presence of residual starch and lower molecular weight oligosaccharides that would be present in the reaction product prior to purification of the synthesized β-1,2-oligoglucan.

### Example 14—αGP Characterization

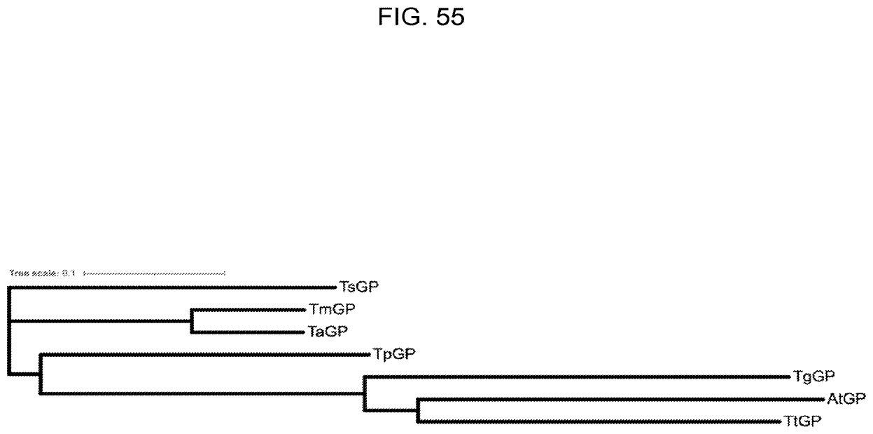

Enzymes from Thermosipho melanesiensis (TmGP) and Thermosynechococcus sp. (TsGP) were recombinantly expressed in E. coli and analysed concerning their biochemical and kinetic features. At the time of the analysis, TmGP and TsGP were the most identical to TaGP compared to all putative a-GPs enzyme sequences in NCBI database (Available on the World Wide Web at ncbi.nlm.nih.gov), at 84% and 56%, respectively (Table 44 and FIG. 55).

TABLE 44
Percent Identity

| | | | | | | | |
|---|---|---|---|---|---|---|---|
| TpGP | 100% | | | | | | |
| TsGP | 50.18% | 100% | | | | | |
| TaGP | 53.14% | 55.59% | 100% | | | | |
| TmGP | 53.63% | 54.46% | 84.05% | 100% | | | |
| TgGP | 18.49% | 16.9% | 18.17% | 18.47% | 100% | | |
| TtGP | 21.88% | 21.44% | 22.17% | 21.44% | 40.28% | 100% | |
| AtGP | 18.71% | 18.57% | 17.89% | 18.44% | 35.79% | 44.92% | 100% |
| | TpGP | TsGP | TaGP | TmGP | TgGP | TtGP | AtGP |

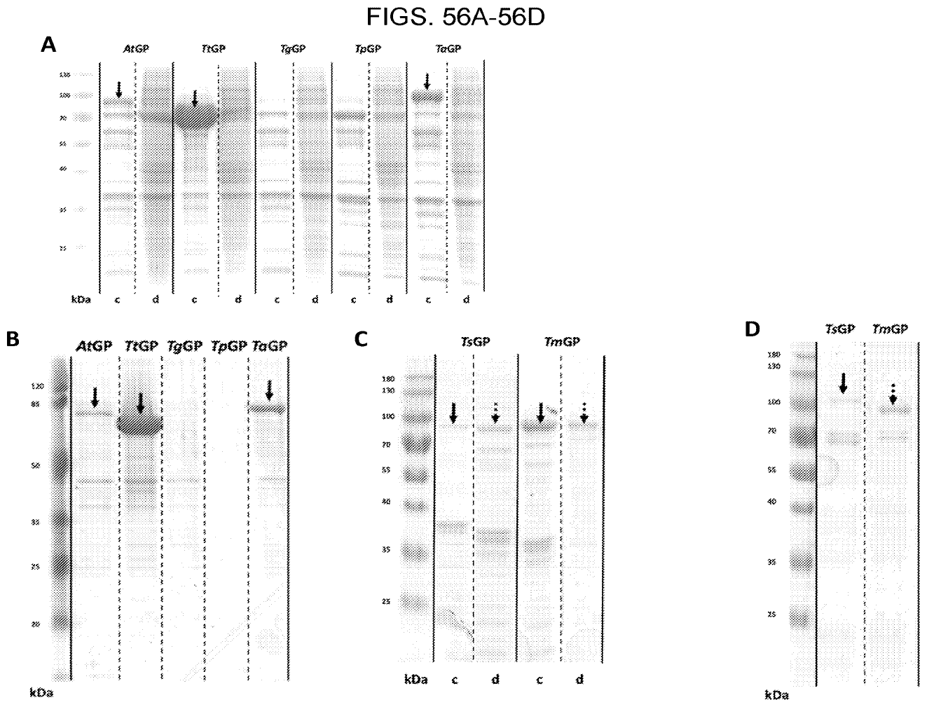

For a detailed biochemical and kinetic characterization, TsGP and TmGP were purified by His-tag affinity chromatography (FIG. 56D). As expected, the enzymes displayed a single protein band on the SDS-PAGE gel with an estimated molecular weight (MW) of 99 and 96 kDa, respectively (Table 45). The temperature optimum of TmGP (60° C.) was found to be similar to that of TaGP. TmGP has a very low tolerance to changes in temperature since it preserves more than 50% of its specific activity at temperatures between 55 and 60° C. (Table 45). TsGP has the lowest Tₒₚₜ=50° C. of all a-GPs analysed in this work (Table 45). In contrast to TmGP, the thermostability assessment of TsGP by SDS-PAGE did not result in a strong protein band after 1 h incubation at 60° C. (FIG. 56C). pH Optimum of TmGP is found to be at pH 6.5, lower than that of TaGP (pHₒₚₜ=8). TmGP, however, retained >50% of its activity at pH 8 (Table 45). In Table 45, pH and temperature profiles were determined using 50 mM phosphate buffer and 2% maltodextrin mixture as substrates, the theoretical molecular weight was calculated using the ExPSAY server ProtParam tool, concentrations of enzymes purified by affinity chromatography was determined by the protein A280 method.

TABLE 45

| | | | Optimal | ~T range | | | |
|---|---|---|---|---|---|---|---|
| | Theoretical | Estimated | activity | (° C.) | Optimal | pH range | Specific |
| | MW | yield | temperature | (>50% | activity | (>50% | activity |
| Enzyme | (kDa) | (mg · L⁻¹) | (° C.) | act.) | pH | act.) | (U · mg⁻¹) |
| TaGP | 102 | 11 | 65 | 50-70 | 8 | 7-8.5 | 132 ± 8 |
| TmGP | 96 | 6 | 60 | 55-60 | 6.5 | 6-8 | 53 ± 2 |
| TsGP | 99 | 2 | 50 | 45-55 | 7.5 | 6-8 | 5 ± 0.2 |

The specific activity of TmGP is 2.5-fold lower compared to TaGP (Table 45). In addition, TmGP was obtained in an almost 2-fold lower yield than TaGP (Table 45). The affinity of TmGP for maltodextrins is equal to that of TaGP, whilst the catalytic efficiency is 1.7-fold lower (Table 46). Contrary to TaGP, TmGP is slightly inhibited by high maltodextrin concentrations with a Kᵢ value of around 53% maltodextrins (Table 46). TsGP, though it has the lowest Michaelis-Menten constant of 0.03 mM maltodextrins compared to all α-GPs analysed in this work, expressed in a meagre yield of 2 mg·L⁻¹ soluble protein and has a low specific activity of 5 U·mg⁻¹, 26-fold lower than that of TmGP (Table 45).

TABLE 46

| | Kₘ | Kₘ | k_{cat} | k_{cat}/Kₘ | Kᵢ | Kᵢ |
|---|---|---|---|---|---|---|
| Enzyme | (%) ^{b} | (mM) | (s⁻¹) | (s⁻¹ · mM⁻¹) | (%) | (mM) |
| TaGP | 0.2 ± 0.04 | 0.07 ± 0.01 | 178 ± 3.4 | 2543 ± 340 | — | — |
| TmGP | 0.2 ± 0.04 | 0.06 ± 0.01 | 91 ± 4 | 1516 ± 400 | 53 ± 12 | 16 ± 4 |
| TsGP | 0.1 ± 0.01 | 0.03 ± 0.003 | 7 ± 0.1 | 233 ± 33 | — | — |

### Example 15—β-Glucan Structural Analysis

1,2-β-glucan compositions were prepared by adding 0.7 mg of either enzyme B7 or enzyme B13 to a solution of 650 mM G1P and 5 mM sophorose in 50 mM MOPS buffer pH 7. The solution was held at 35° C. and shaken for 24 hours. After 24 hours, the reaction was stopped by heating for 5 minutes at 100° C. After heat deactivation of the enzyme, enzyme and residual salt were removed by centrifugation and mixed bed resin treatment. The β-glucan composition was further purified by isopropanol precipitation and vacuum oven drying. Glucose and fructose were removed from the dried composition by dialysis after dissolving in water. The dialysis purified β-glucan composition was then freeze-dried, and oligosaccharide composition of the freeze-dried product was analysed using high-performance liquid chromatography (HPLC) and low molecular weight gas phase chromatography (LMW GPC). LMW GPC Results for the β-glucan composition produced by enzyme B7 are reported in Table 47 and results for the β-glucan composition produced by enzyme B13 are reported in Table 48. HPLC data is reported in Table 49.

TABLE 47
B7 β-glucan product LMW GPC

| Sample: | B7 | B7 |
|---|---|---|
| | Injection 1 | Injection 2 |
| Mn | 2850 | 2818 |
| Mw | 2953 | 2928 |
| Polydispersity (Mw/Mn) | 1.04 | 1.0 |

| | | |
|---|---|---|
| DP Range | Mw Range | Slicing (Area %) |

| | | | |
|---|---|---|---|
| 1−>5 | <909 | 0.0 | 0.1 |
| 6−>9 | 909−>1557 | 0.3 | 0.4 |
| 10−>19 | 1557−>3177 | 67.3 | 68.0 |
| 20−>45 | 3177−>7389 | 32.3 | 31.5 |
| 46−>125 | 7389−>20349 | 0.0 | 0.0 |
| 126−>280 | 20349−>45459 | 0.0 | 0.0 |
| 281−>600 | 45459−>97299 | 0.0 | 0.0 |
| 601−>1500 | 97299−>243828 | 0.0 | 0.0 |
| >1500 | >243828 | 0.0 | 0.0 |

TABLE 48
B13 β-glucan product LMW GPC

| Sample: | B13 |
|---|---|
| | Injection 1 |
| Mn | 8341 |
| Mw | 9629 |
| Polydispersity (Mw/Mn) | 1.2 |

| | | Slicing |
|---|---|---|
| DP Range | Mw Range | (Area %) |
| 1−>5 | <909 | 0.0 |
| 6−>9 | 909−>1557 | 0.1 |
| 10−>19 | 1557−>3177 | 0.9 |
| 20−>45 | 3177−>7389 | 24.5 |
| 46−>125 | 7389−>20349 | 73.9 |
| 126−>280 | 20349−>45459 | 0.6 |
| 281−>600 | 45459−>97299 | 0.0 |
| 601−>1500 | 97299−>243828 | 0.0 |
| >1500 | >243828 | 0.0 |

TABLE 49
HPLC Oligosaccharide Analysis

| Sample | B7 | B13 |
|---|---|---|
| DP n | 100% | 100% |
| DP 10 | 0% | 0% |
| DP 9 | 0% | 0% |
| DP 8 | 0% | 0% |
| DP 7 | 0% | 0% |
| DP 6 | 0% | 0% |
| DP 5 | 0% | 0% |
| DP 4 | 0% | 0% |
| DP 3 | 0% | 0% |
| DP 2 | 0% | 0% |
| Dextrose | 0% | 0% |

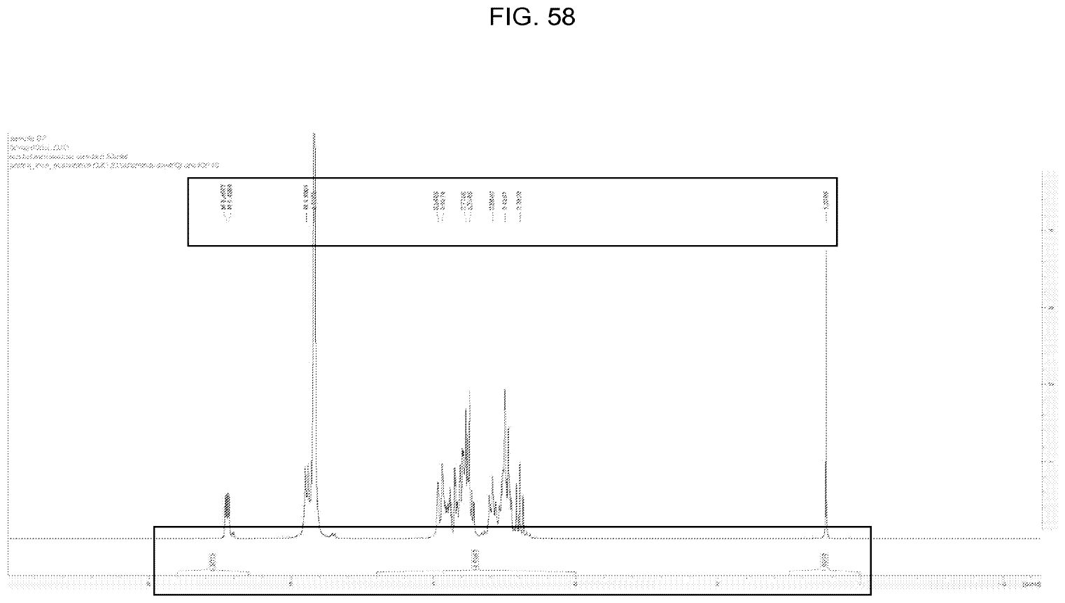

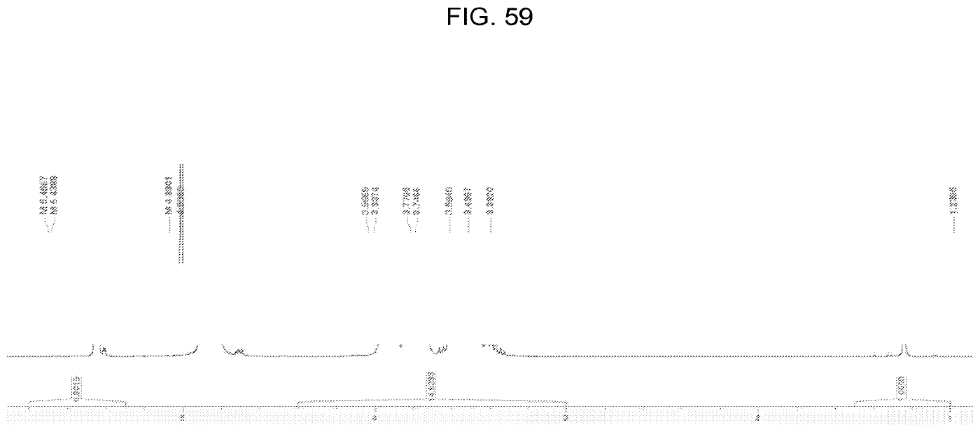

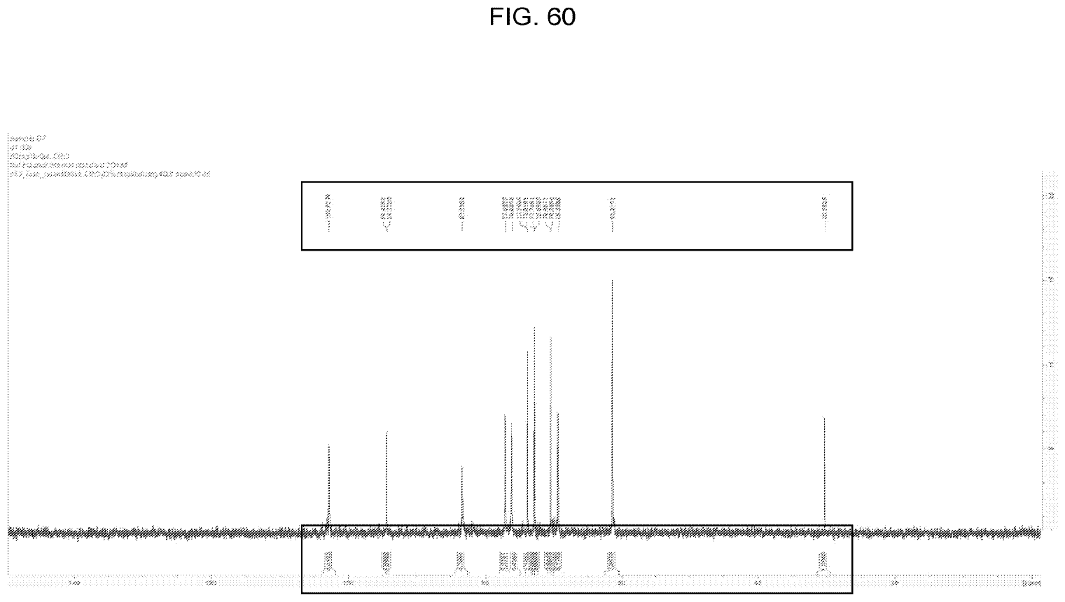

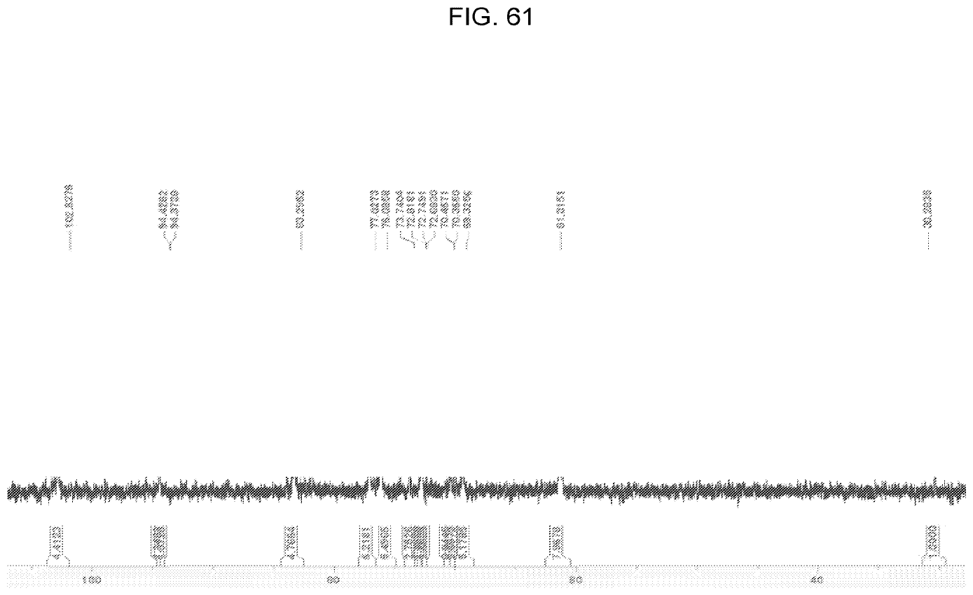

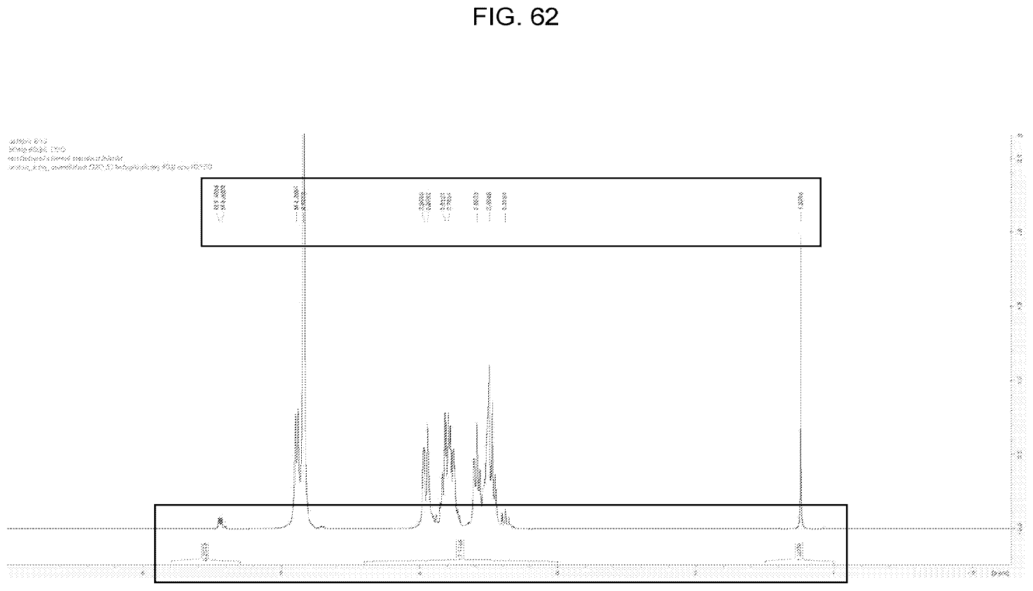

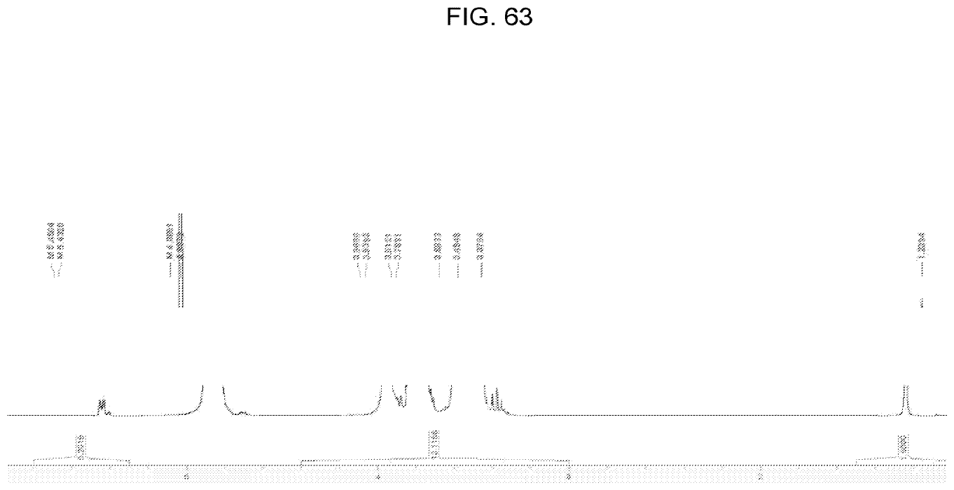

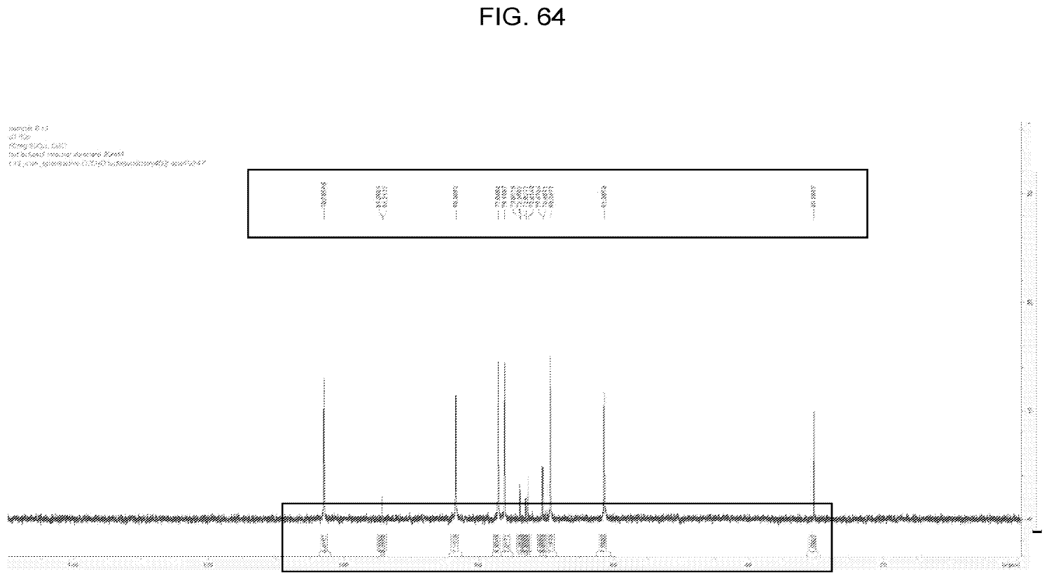

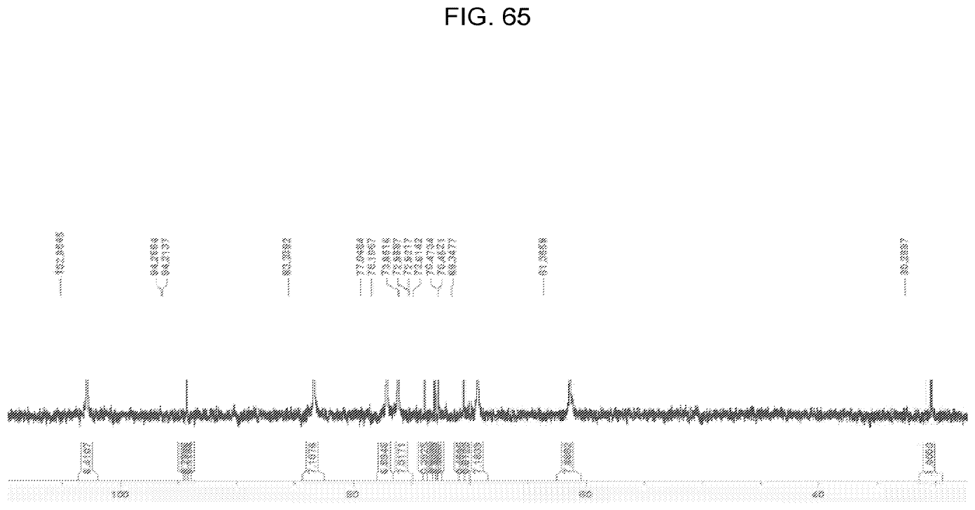

Additionally, both the B7 and B13 β-glucan products were analyzed by NMR using the method outlined in Example 10. Structural identity of β-1,2-glucan produced from either the B7 or B13 enzymes was confirmed by 1H- and 13C-NMR. (FIGS. 58-65).

1. A method for the production of 1,2-beta-oligoglucans, the method comprising:
contacting α-D-glucose-1-phosphate (G1P) with a beta(β)-glucan-phosphorylase (βGP) to produce 1,2-beta-oligoglucans, wherein the βGP has an amino acid sequence at least 80% identical to at least one of SEQ ID NOs: 10, 11, 13, 15, and 16.

2. The method of claim 1, additionally comprising the step of contacting a substrate with an alpha(α)-glucan-phosphorylase (αGP) in the presence of inorganic phosphate (e.g., sodium phosphate or potassium phosphate) to produce the glucose-1-phosphate, wherein the αGP has an amino acid sequence at least 80% identical to at least one of SEQ ID NOs: 1, 2, 3, 4, 5, or 18.

3. The method of claim 2, wherein the substrate has a degree of polymerization (DP) equal to or greater than 4.

4. The method of claim 2, wherein the substrate is selected from the group consisting of maltodextrin, starch liquefact, trehalose, sucrose, cellulose, cellodextrins, cellobiose, and combinations thereof.

5. The method of claim 2, wherein the βGP G1P contacting step is carried out at a pH between 6.0 and 7.5 and/or the substrate αGP contacting step is carried out at a pH between 6.5 and 8.0.

6. A composition comprising
i) a beta(β)-glucan-phosphorylase (βGP) having an amino acid sequence at least 80% identical to at least one of SEQ ID NOs: 10, 11, 13, 15, and 16;
ii) α-D-glucose-1-phosphate (G1P); and
iii) a primer molecule.

7. The composition of claim 6, additionally comprising a 1,2-beta-oligoglucan, a phosphatase inhibitor (e.g., sodium molybdate), a buffer, and/or a reducing agent.

8. The composition of claim 6, wherein the primer molecule is selected from the group consisting of D-glucose, sophorose, laminaribiose, cellobiose, gentiobiose, and combinations thereof.

9. (canceled)

10. The method of claim 2,
wherein the αGP has an amino acid sequence at least 90% identical to at least one of SEQ ID NOs: 1, 2, or 5.

11. The composition or method of claim 1,
wherein the βGP has an amino acid sequence at least 90% identical to SEQ ID NO:11, 13, or 16.

12. A 1,2-beta-olioglucan composition produced by the method of claim 1, wherein the composition has a polydispersity between 2 and 40, a degree of polymerization (DP) of about 6-150, and a viscosity between 800 and 1200 mPas at 50° C.

13. (canceled)

14. (canceled)

15. A vector comprising a nucleic acid encoding a polypeptide at least 80% identical to at least one of SEQ ID NOs: 5, 11, 13, and 16.

16. (canceled)

17. A cell comprising the vector of claim 15.
